(19) United States
(12) Patent Application Publication
(10) Pub. No.: US 2024/0244959 A1
(43) Pub. Date: Jul. 18, 2024

(54) ORGANIC LIGHT EMITTING ELEMENT AND DISPLAY DEVICE

(71) Applicant: LG DISPLAY CO., LTD., Seoul (KR)

(72) Inventors: Jicheol SHIN, Gyeongsangnam-do (KR); Su Bin AHN, Gyeonggi-do (KR)

(73) Assignee: LG DISPLAY CO., LTD., Seoul (KR)

(21) Appl. No.: 18/512,662

(22) Filed: Nov. 17, 2023

(30) Foreign Application Priority Data
Dec. 31, 2022 (KR) 10-2022-0191361

(51) Int. Cl. H10K 85/60; C07C 255/47; C07C 255/52; C07D 213/61; C09K 11/06

(52) U.S. Cl. CPC H10K 85/615; C07C 255/47; C07C 255/52; C07D 213/61; C09K 11/06; H10K 85/654; H10K 50/17

(57) ABSTRACT

Embodiments of the disclosure relate to an organic light emitting element and a display device. Specifically, there may be provided an organic light emitting element including a compound represented by chemical formula 1 to provide excellent efficiency, long lifespan or low driving voltage and a display device including the organic light emitting element.

### CROSS-REFERENCE TO RELATED APPLICATION

This application claims priority from Korean Patent Application No. 10-2022-0191361, filed on Dec. 31, 2022.

### BACKGROUND

### Technical Field

Embodiments of the disclosure relate to an organic light emitting element and a display device.

### Discussion of the Related Art

In general, organic light emission refers to a phenomenon in which electric energy is converted into light energy by an organic material. The organic light emitting element refers to a light emitting element using the organic light emission phenomenon. The organic light emitting element has a structure including an anode, a cathode, and an organic material layer disposed therebetween.

The organic material layer may have a multilayer structure composed of different materials to increase the efficiency and stability of the organic light emitting element and may include a light emitting layer (also referred to as an emission material layer (EML)).

The lifespan and efficiency are the most important issues with organic light emitting elements. The efficiency, lifespan, and driving voltage are related to each other. If the efficiency is increased, the driving voltage is relatively decreased, so that the crystallization of the organic material by the Joule heating during driving is reduced, leading to an increase in lifespan.

The role of the light emitting layer EML is important to enhance the light emitting properties of the organic light emitting element and increase the lifespan. In particular, to have high-efficiency characteristics, the host material of the light emitting layer is required to have a high triplet energy level, and further a sufficient stability of the material is needed.

### SUMMARY

Accordingly, embodiments of the present disclosure are directed to an organic light emitting element and a display device that substantially obviate one or more of the problems due to limitations and disadvantages of the related art.

Additional features and aspects will be set forth in the description that follows, and in part will be apparent from the description, or may be learned by practice of the inventive concepts provided herein. Other features and aspects of the inventive concepts may be realized and attained by the structure particularly pointed out in the written description, or derivable therefrom, and the claims hereof as well as the appended drawings.

To achieve these and other aspects of the inventive concepts, as embodied and broadly described herein, an organic light emitting element may comprise an organic material layer including a hole injection layer and a charge generation layer between an anode and a cathode. The hole injection layer and the charge generation layer are layers closely related to hole injection and movement characteristics that determine the characteristics of the device, and organic electron acceptor compounds may be used for efficient hole generation, injection and movement. Since the organic electron acceptor compound includes a strong electron withdrawing group (EWG), when the hole injection layer is doped with it, it may withdraw electrons from a high occupied molecular orbital (HOMO) energy level of the adjacent hole transport layer to a low occupied molecular orbital (LUMO) energy level of the organic electron acceptor compound to generate holes and inject the holes into the hole transport layer. Therefore, organic electron acceptor compounds may be designed to include a number of strong electron withdrawing groups for efficient hole generation, injection and transfer.

Organic electron acceptor compounds may contain strong electron withdrawing groups to have low LUMO energy levels. However, since the organic electron acceptor compounds typically have a low miscibility with the hole transporting compound, a high driving voltage and low luminous efficiency may occur due to inefficient charge injection and transfer characteristics. Accordingly, the inventors of the disclosure have invented an organic light emitting element and a display device that may have high efficiency, long lifespan and/or low driving voltage.

Embodiments of the disclosure may provide an organic light emitting element and a display device that may have high efficiency, long lifespan, and/or low driving voltage.

Embodiments of the disclosure may provide an organic light emitting element including a first electrode, a second electrode, and an organic material layer positioned between the first electrode and the second electrode.

The organic material layer may include the compound represented by chemical formula 1 described below.

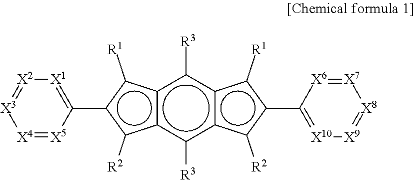

Embodiments of the disclosure may provide a display device including the organic light emitting element described above.

### Effects of the Disclosure

According to embodiments of the disclosure, there may be provided an organic light emitting element having high emission efficiency, long lifespan, and/or low driving voltage.

According to embodiments of the disclosure, there may be provided an organic light emitting element having high emission efficiency, long lifespan and/or low driving voltage by including a layer having excellent hole injection characteristics or electron injection characteristics.

It is to be understood that both the foregoing general description and the following detailed description are exemplary and explanatory and are intended to provide further explanation of the inventive concepts as claimed.

### BRIEF DESCRIPTION OF DRAWINGS

The accompanying drawings, which are included to provide a further understanding of the disclosure and are incorporated in and constitute a part of this application, illustrate embodiments of the disclosure and together with the description serve to explain various principles. In the drawings:

FIG. 1 illustrates a system configuration of a display device according to embodiments of the disclosure;

FIG. 2 illustrates a subpixel circuit of a display device according to embodiments of the disclosure; and

FIGS. 3, 4, 5, 6, 7, 8, 9, and 10 are cross-sectional views schematically illustrating an organic light emitting element according to embodiments of the disclosure.

### DETAILED DESCRIPTION

In the following description of examples or embodiments of the disclosure, reference will be made to the accompanying drawings in which, by way of illustration, specific examples or embodiments are shown that can be implemented by a person skilled in the art, and in which the same reference numerals and signs can be used to designate the same or like components even when they are shown in different of the accompanying drawings. Further, in the following description of examples or embodiments of the disclosure, detailed descriptions of well-known functions and components incorporated herein will be omitted when it is determined that the description of the same may render subject matter described in conjunction with the embodiments of the disclosure less clear. The terms such as “including”, “having”, “containing”, “constituting” “make up of”, and “formed of” used herein are generally intended to allow other components to be added unless the terms are used with the term “only”. As used herein, singular forms are intended to include plural forms unless the context clearly indicates otherwise.

Terms, such as “first”, “second”, “A”, “B”, “(A)”, or “(B)” may be used herein to describe elements of the disclosure. Each of these terms is not used to define essence, order, sequence, or number of elements etc., but is used merely to distinguish the corresponding element from other elements.

When it is mentioned that a first element “is connected or coupled to”, “contacts or overlaps” etc. a second element, it should be interpreted that, not only can the first element “be directly connected or coupled to” or “directly contact or overlap” the second element, but a third element can also be “interposed” between the first and second elements, or the first and second elements can “be connected or coupled to”, “contact or overlap”, etc. each other via a fourth element. Here, the second element may be included in at least one of two or more elements that “are connected or coupled to”, “contact or overlap”, etc. each other.

When time relative terms, such as “after,” “subsequent to,” “next,” “before,” and the like, are used to describe processes or operations of elements or configurations, or flows or steps in operating, processing, manufacturing methods, these terms may be used to describe non-consecutive or non-sequential processes or operations unless the term “directly” or “immediately” is used together with the said relative terms.

In addition, when any dimensions, relative sizes etc. are mentioned, it should be considered that numerical values for an elements or features, or corresponding information (e.g., level, range, etc.) include a tolerance or error range that may be caused by various factors e.g., process factors, internal or external impact, noise, etc.) even when a relevant description is not specified. Further, the term “may” fully encompasses all the meanings of the term “can”.

Hereinafter, various embodiments of the disclosure are described in detail with reference to the accompanying drawings.

As used herein, the term “halo” or “halogen” includes fluorine (F), chlorine (Cl), bromine (Br), and iodine (I), and the like, unless otherwise specified.

As used herein, the term “alkyl” or “alkyl group” may mean a radical of a saturated aliphatic functional group having 1 to 60 carbon atoms linked by a single bond and including a straight chain alkyl group, branched chain alkyl group, cycloalkyl (alicyclic) group, alkyl-substituted cycloalkyl group, or cycloalkyl-substituted alkyl group, unless otherwise specified.

As used herein, the term “haloalkyl group” or “halogenalkyl group” may mean a halogen-substituted alkyl group unless otherwise specified.

As used herein, the term “alkenyl” or “alkynyl” may have a double bond or a triple bond, respectively, and may include a straight or branched chain group and may have 2 to 60 carbon atoms unless otherwise specified.

As used herein, the term “cycloalkyl” may refer to an alkyl forming a ring having 3 to 60 carbon atoms, unless otherwise specified.

As used herein, the term “alkoxy group” or “alkyloxy group” refers to an alkyl group to which an oxygen radical is bonded, and may have 1 to 60 carbon atoms unless otherwise specified.

As used herein, the term “alkenoxyl group”, “alkenoxy group”, “alkenyloxyl group”, or “alkenyloxy group” refers to an alkenyl group to which an oxygen radical is attached, and may have 2 to 60 carbon atoms unless otherwise specified.

As used herein, the terms “aryl group” and “arylene group” each refer to a group that may have 6 to 60 carbon atoms unless otherwise specified, but are not limited thereto. In the disclosure, the aryl group or the arylene group may include a monocyclic type, a ring assembly, a fused polycyclic system, a spiro compound, and the like. For example, the aryl group includes, but is not limited to, phenyl, biphenyl, naphthyl, anthryl, indenyl, phenanthryl, triphenylenyl, pyrenyl, peryleneyl, chrysenyl, naphthacenyl, or fluoranthenyl. The term “naphthyl” may relate to 1-naphthyl and 2-naphthyl, and the term “anthryl” may relate to 1-anthryl, 2-anthryl and 9-anthryl.

In the disclosure, the term “fluorenyl group” or “fluorenylene group” may refer to a monovalent or divalent functional group, respectively, of fluorene, unless otherwise specified. The “fluorenyl group” or “fluorenylene group” may mean a substituted fluorenyl group or a substituted fluorenylene group. “Substituted fluorenyl group” or “substituted fluorenylene group” may refer to a monovalent or divalent functional group of substituted fluorene. “Substituted fluorene” may mean that at least one of the following substituents R, R′, R″ and R′″ is a functional group other than hydrogen. It may include a situation where R and R′ are bonded to each other to form a spiro compound together with the carbon to which they are bonded.

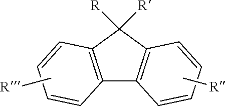

As used herein, the term “spiro compound” refers to a compound that has a ‘spiro union’, and the term “spiro union” means a union formed from two rings that share only one atom. In this case, the atom shared by the two rings may be referred to as a ‘spiro atom’.

As used herein, the term “heterocyclic group” may include not only an aromatic ring, such as a “heteroaryl group” or “heteroarylene group” but also a non-aromatic ring and, unless otherwise specified, means a ring with 2 to 50 carbon atoms and one or more heteroatoms, but is not limited thereto. As used herein, the term “heteroatom” refers to N, O, S, P or Si unless otherwise specified, and the term “heterocyclic group” may designate a monocyclic group containing a heteroatom, a ring assembly, a fused polycyclic system, or a spiro compound.

The “heterocyclic group” may include a ring containing SO2 instead of carbon forming the ring. For example, the “heterocyclic group” may include the following compounds.

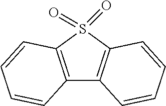 [SMILES: O=S1(=O)c2ccccc2-c2ccccc21]

As used herein, the term “ring” may include monocycles and polycycles, may include hydrocarbon rings as well as heterocycles containing at least one heteroatom, or may include aromatic and non-aromatic rings.

As used herein, the term “polycycle” may include ring assemblies, fused polycyclic systems, and/or spiro compounds, may include aromatic as well as non-aromatic compounds, and/or may include heterocycles containing at least one heteroatom as well as hydrocarbon rings.

As used herein, the term “aliphatic ring group” refers to a cyclic hydrocarbon other than the aromatic hydrocarbon, may include a monocyclic type, a ring assembly, a fused polycyclic system, and a spiro compound and, unless otherwise specified, may designate a ring having 3 to 60 carbon atoms. For example, a fusion of benzene, which is an aromatic ring, and cyclohexane, which is a non-aromatic ring, also corresponds to an aliphatic ring.

As used herein, the term “alkylsilyl group” may refer to a monovalent substituent in which three alkyl groups are bonded to a Si atom.

As used herein, the term “arylsilyl group” may refer to a monovalent substituent in which three aryl groups are bonded to a Si atom.

As used herein, the term “alkylarylsilyl group” may refer to a monovalent substituent in which one alkyl group and two aryl groups are bonded to a Si atom or two alkyl groups and one aryl group are bonded to the Si atom.

As used herein, the term “ring assembly” means that two or more ring systems (single or fused ring systems) are directly connected to each other through single or double bonds. For example, in the case of an aryl group, a biphenyl group or a terphenyl group may be a ring assembly but is not limited thereto.

As used herein, the term “fused polycyclic system” refers to a type of fused rings sharing at least two atoms. For example, in the case of an aryl group, a naphthalenyl group, a phenanthrenyl group, or a fluorenyl group may be a fused polycyclic system, but is not limited thereto.

When prefixes are named successively, it may mean that the substituents are listed in the order specified first. For example, an arylalkoxy group may mean an alkoxy group substituted with an aryl group, an alkoxycarbonyl group may mean a carbonyl group substituted with an alkoxy group, and an arylcarbonylalkenyl group may mean an alkenyl group substituted with an arylcarbonyl group. The arylcarbonyl group may be a carbonyl group substituted with an aryl group.

Unless otherwise explicitly stated, in the term “substituted” or “unsubstituted” as used herein, “substituted” may mean being substituted with one or more substitutents selected from the group consisting of halogen, an amino group, a nitrile group, a nitro group, a C1-C20 alkyl group, a C1-C20 alkoxy group, a C1-C20 alkylamine group, a C1-C20 alkylthiophene group, a C6-C20 arylthiophene group, a C2-C20 alkenyl group, a C2-C20 alkynyl group, a C3-C20 cycloalkyl group, a C6-C20 aryl group, a C8-C20 arylalkenyl group, a silane group, a boron group, a germanium group, and a C2-C20 heterocyclic group including at least one heteroatom selected from the group consisting of O, N, S, Si and P, but is not limited to these substituents.

In the disclosure, the ‘functional group names’ corresponding to the aryl group, arylene group, and heterocyclic group provided as examples of the symbols and their substituents may be described with ‘the names of the functional groups reflecting the valence’, but may also be described with ‘the names of the parent compounds’. For example, in the case of ‘phenanthrene’, which is a type of aryl group, its name may be specified with its group identified, such as ‘phenanthryl (group)’ for the monovalent group, and ‘phenanthrylene (group)’ as the divalent group, but may also be specified as ‘phenanthrene’, which is the name of the parent compound, regardless of the valence. Similarly, pyrimidine may be specified as ‘pyrimidine’ regardless of the valence or may also be specified as pyrimidinyl (group) for the monovalent group and as pyrimidylene (group) for the divalent group. Therefore, in the disclosure, when the type of the substituent is specified with the name of the parent compound, it may mean an n-valent ‘group’ formed by detachment of the hydrogen atom bonded to a carbon atom and/or a heteroatom of the parent compound.

Further, unless explicitly stated, the formulas used in the disclosure may be applied in the same manner as the definition of the substituent by the index definition of the following formulas.

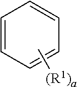

When a is 0, it means that the substituent R¹ does not exist, meaning that hydrogen is bonded to each of the carbon atoms forming the benzene ring. In this case, the chemical formula or chemical compound may be specified without expressing the hydrogen bonded to the carbon. Further, when a is 1, one substituent R¹ is bonded to any one of the carbon atoms forming the benzene ring, and when a is 2 or 3, it may be bonded as follows. When a is an integer of 4 to 6, it is bonded to the carbon of the benzene ring in a similar manner, and when a is an integer of 2 or more, R¹ may be identical or different.

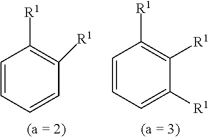

In the disclosure, when substituents are bonded to each other to form a ring, it may mean that adjacent groups are bonded to each other to form a monocycle or fused polycycle, and the monocycle or fused polycycle may include heterocycles containing at least one heteroatom as well as hydrocarbon rings and may include aromatic and non-aromatic rings.

In the disclosure, organic light emitting element may mean a component (s) between an anode and a cathode of an electronic device or an organic light emitting diode including an anode, a cathode, and component (s) positioned therebetween.

In some cases, in the disclosure, organic light emitting element may mean an organic light emitting diode and a panel including the same, or an electronic device including the panel and circuitry. The electronic device may include, e.g., a display device, a lighting device, a solar cell, a portable or mobile terminal (e.g., a smart phone, a tablet, a PDA, an electronic dictionary, or PMP), a navigation terminal, a game device, various TVs, and various computer monitors but, without being limited thereto, may include any type of device including the component (s).

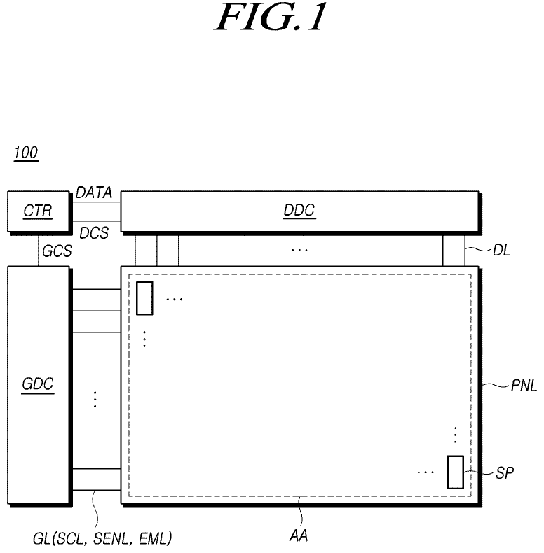

FIG. 1 illustrates a system configuration of a display device 100 according to embodiments of the disclosure.

Referring to FIG. 1, a display device 100 according to embodiments of the disclosure includes a display panel PNL in which a plurality of data lines DL and a plurality of gate lines GL are disposed, and a plurality of subpixels SP defined by the plurality of data lines DL and the plurality of gate lines GL are arranged, a data driving circuit DDC for driving the plurality of data lines DL, a gate driving circuit GDC for driving the plurality of gate lines GL, and a controller CTR for controlling the data driving circuit DDC and the gate driving circuit GDC.

The controller CTR supplies various control signals DCS and GCS to the data driving circuit DDC and the gate driving circuit GDC to control the data driving circuit DDC and the gate driving circuit GDC.

The data driving circuit DDC receives the image data DATA from the controller CTR and supplies data voltage to the plurality of data lines DL, thereby driving the plurality of data lines DL. Herein, the data driving circuit DDC is also referred to as a ‘source driving circuit’.

The gate driving circuit GDC sequentially drives the plurality of gate lines GL by sequentially supplying scan signals to the plurality of gate lines GL. Herein, the gate driving circuit GDC is also referred to as a ‘scan driving circuit’.

The gate driving circuit GDC sequentially supplies scan signals of ‘On voltage’ or ‘Off voltage’ to the plurality of gate lines GL under the control of the controller CTR.

When a specific gate line is opened by the gate driving circuit GDC, the data driving circuit DDC converts the image data DATA received from the controller CTR into an analog data voltage and supplies the analog data voltage to the plurality of data lines DL.

The data driving circuit DDC may be positioned on only one side (e.g., the top or bottom side) of the display panel PNL and, in some cases, the driving circuit may be positioned on each of two opposite sides (e.g., both the top and bottom sides) of the display panel PNL depending on, e.g., driving schemes or panel designs.

The gate driving circuit GDC may be positioned on only one side (e.g., the left or right side) of the display panel PNL and, in some cases, the gate driving circuit GDR may be positioned on each of two opposite sides (e.g., both the left and right sides) of the display panel PNL depending on, e.g., driving schemes or panel designs.

The display device 100 according to embodiments of the disclosure may be an organic light emitting display device, a liquid crystal display device, a plasma display device, and the like.

When the display device 100 according to the embodiments of the disclosure is an organic light emitting display device, each subpixel SP arranged on the display panel PNL may be composed of a circuit element such as an organic light emitting diode (OLED) that is a self-luminous element, and a driving transistor for driving the OLED.

The type and number of circuit elements constituting each subpixel SP may be varied depending on functions to be provided and design schemes.

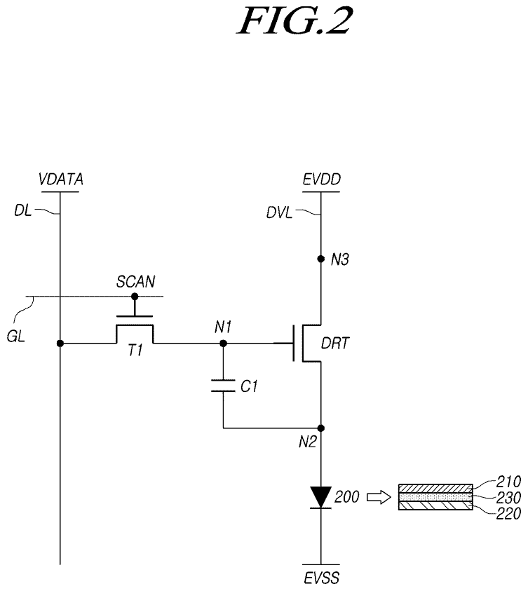

FIG. 2 illustrates a subpixel circuit of a display device according to embodiments of the disclosure.

Referring to FIG. 2, each subpixel SP may basically include an organic light emitting element 200 and a driving transistor DRT for driving the organic light emitting element 200.

Each subpixel SP may further include a first transistor T1 to transfer data voltage, VDATA, to a first node N1, which corresponds to a gate node of the driving transistor DRT, and a storage capacitor C1 to maintain the data voltage, VDATA, corresponding to an image signal voltage or a voltage corresponding to the data voltage VDATA for the time of one frame.

The organic light emitting element 200 may include a first electrode 210 (anode electrode or cathode electrode), an organic material layer 230, and a second electrode 220 (cathode electrode or anode electrode).

As an example, a base voltage EVSS may be applied to the second electrode 220 of the organic light emitting diode 200.

The driving transistor DRT supplies a driving current to the organic light emitting diode 200, thereby driving the organic light emitting diode 200.

The driving transistor DRT includes the first node N1, second node N2, and third node N3.

The first node N1 of the driving transistor DRT is a node corresponding to the gate node and may be electrically connected with the source node or drain node of the first transistor T1.

The second node N2 of the driving transistor DRT may be electrically connected with the first electrode 210 of the organic light emitting diode 200, and may be a source node or a drain node.

The third node N3 of the driving transistor DRT may be a node to which driving voltage EVDD is applied, may be electrically connected with a driving voltage line DVL for supplying the driving voltage EVDD, and may be the drain node or source node.

The first transistor T1 may be electrically connected between the data line DL and the first node N1 of the driving transistor DRT, and may be controlled by receiving the scan signal SCAN through the gate line and the gate node.

The storage capacitor C1 may be electrically connected between the first node N1 and second node N2 of the driving transistor DRT.

The storage capacitor C1 is an external capacitor intentionally designed to be outside the driving transistor DRT, but not a parasite capacitor (e.g., Cgs or Cgd) which is an internal capacitor present between the first node N1 and the second node N2 of the driving transistor DRT.

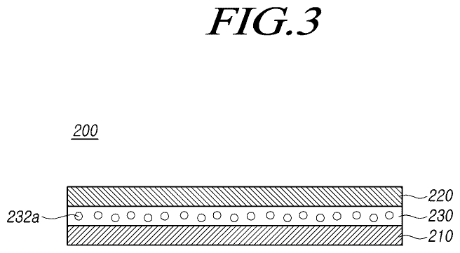

FIG. 3 is a cross-sectional view schematically illustrating an organic light emitting element according to embodiments of the disclosure.

The organic light emitting element 200 according to embodiments of the disclosure includes a first electrode 210, a second electrode 220, and an organic material layer 230 positioned between the first electrode 210 and the second electrode 220.

For example, the first electrode 210 may be the anode electrode, and the second electrode 220 may be the cathode electrode.

For example, the first electrode 210 may be a transparent electrode, and the second electrode 130 may be a reflective electrode. In another example, the first electrode 210 may be a reflective electrode, and the second electrode 130 may be a transparent electrode.

The organic material layer 230 is a layer positioned between the first electrode 210 and the second electrode 220 and including an organic material and may be composed of a plurality of layers.

The organic material layer 230 includes a compound 232a represented by chemical formula 1. The compound 232a is described below in detail. As the organic material layer 230 includes the compound 232a represented by chemical formula 1 described above, the organic light emitting element may have high efficiency, long lifespan, and/or low driving voltage.

The organic material layer 230 may include a light emitting layer. The organic layer 230 may further include at least one of a hole injection layer, a hole transport layer, an electron transport layer, and an electron injection layer.

For example, the organic material layer 230 may include a hole injection layer positioned on the first electrode 210, a hole transport layer positioned on the hole injection layer, a light emitting layer positioned on the hole transport layer, an electron transport layer positioned on the light emitting layer, and an electron injection layer positioned on the electron transport layer. In such an example, the first electrode 210 may be the anode electrode, and the second electrode 220 may be the cathode electrode.

The light emitting layer is a layer in which as holes and electrons transferred from the first electrode 210 and the second electrode 130 meet to emit light and may include, e.g., a host material and a dopant.

In other words, the organic material layer 230 may include, e.g., a hole injection layer, a hole transport layer, a light emitting layer, an electron transport layer, and an electron injection layer. The hole injection layer may be positioned on the first electrode 210 as the anode electrode. The hole transport layer may be positioned on the hole injection layer. The light emitting layer may be positioned on the hole transport layer. The electron transport layer may be positioned on the light emitting layer. The electron injection layer may be positioned on the electron transport layer.

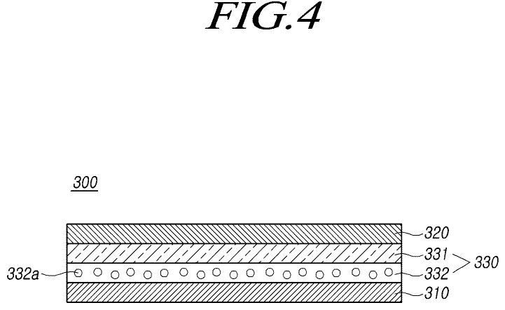

FIG. 4 is a cross-sectional view schematically illustrating an organic light emitting element 400 according to embodiments of the disclosure.

The organic light emitting element 300 may include a first electrode 310, a second electrode 320, and an organic material layer 330 positioned between the first electrode 310 and the second electrode 320.

The organic material layer 330 may include a light emitting layer 331 and a first layer 332.

The first layer 332 may include a compound 332a represented by chemical formula 1. The compound 332a is described below in detail. As the first layer 332 includes the compound 332a represented by chemical formula 1 described above, the organic light emitting element may have high efficiency, long lifespan, and/or low driving voltage.

The organic material layer 330 may include a light emitting layer 331 and a first layer 332. For example, the first electrode 310 may be an anode electrode, and the first layer 332 may be positioned between the first electrode 310 and the light emitting layer 331.

The first layer 332 may be, e.g., a hole injection layer or a hole transport layer. For example, the first layer 332 may be a hole injection layer. As the hole injection layer includes the compound 332a represented by chemical formula 1 described above, the organic light emitting element may have high efficiency, long lifespan, and/or low driving voltage.

The first layer 332 may include the compound 332a represented by chemical formula 1 described above as a dopant. The above-described compound 332a may be included in the first layer 332 as a p dopant. For example, the first layer 332 may be formed by doping with 1 wt % to 40 wt % of the compound 332a represented by chemical formula 1 described above. Alternatively, the first layer 332 may essentially consist of the compound 332a represented by chemical formula 1.

The organic material layer 330 may include, e.g., a first layer 332 that is a hole injection layer, a hole transport layer, a light emitting layer, an electron transport layer, and an electron injection layer. The hole injection layer may be positioned on the first electrode 210 as the anode electrode. The hole transport layer may be positioned on the hole injection layer. The light emitting layer may be positioned on the hole transport layer. The electron transport layer may be positioned on the light emitting layer. The electron injection layer may be positioned on the electron transport layer.

The hole injection layer may include an amine-based compound. For example, the hole injection layer may include one or more of HATCN (1,4,5,8,9,11-hexaazatriphenylenehexacarbonitile) and NPD (N,N′-di(1-naphthyl)-N,N′-diphenyl-(1,1′-biphenyl)-4,4′-diamine). However, the material for the hole injection layer is not limited to those described above, and may include other compounds that may be used as hole injection materials in the field of organic light emitting elements.

The hole transport layer may include an amine-based compound. For example, the hole transport layer may include one or more of HATCN (1,4,5,8,9,11-hexaazatriphenylenehexacarbonitile) and NPD (N,N′-di(1-naphthyl)-N,N′-diphenyl-(1,1′-biphenyl)-4,4′-diamine). However, the material for the hole transport layer is not limited to those described above, and may include other compounds that may be used as hole transport materials in the field of organic light emitting elements.

The light emitting layer may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis (N-carbazolyl)-1,1′-biphenyl). The iridium-based compound may be Ir(ppy)₃(tris(2-phenylpyridine)iridium(III)). However, the material for the light emitting layer is not limited to those described above, and may include other compounds that may be used as light emitting layer materials in the field of organic light emitting elements.

The electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1-H-benzimidazole)). However, the material for the electron transport layer is not limited to those described above, and may include other compounds that may be used as electron transport materials in the field of organic light emitting elements.

The electron injection layer may include at least one of an azine-based compound and an imidazole-based compound. For example, the electron injection layer may include one or more of LiF and LiQ. However, the material for the electron injection layer is not limited to those described above, and may include other compounds that may be used as electron injection materials in the field of organic light emitting elements.

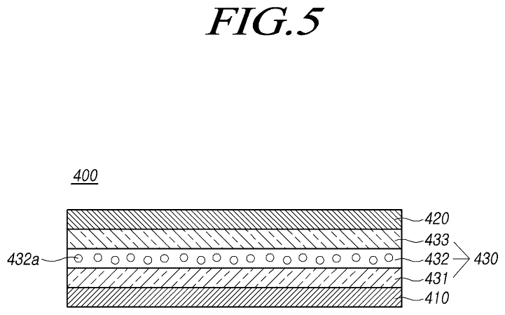

FIG. 5 is a cross-sectional view schematically illustrating an organic light emitting element 400 according to embodiments of the disclosure.

The organic light emitting element 400 may include a first electrode 410, a second electrode 420, and an organic material layer 430 positioned between the first electrode 410 and the second electrode 420.

The organic material layer 430 may include a first light emitting layer 431, a second light emitting layer 433, and a first layer 432 positioned between the first light emitting layer 431 and the second light emitting layer 433. In other words, the organic light emitting element 400 may be a tandem type organic light emitting element including two or more light emitting layers. The tandem type organic light emitting element may include a plurality of stacks each including a light emitting layer. For example, the tandem type organic light emitting element may include a first stack including a first light emitting layer 431 and a second stack including a second light emitting layer 433. In this example, the first stack may include additional functional layers in addition to the first light emitting layer 431. Further, the second stack may include additional functional layers in addition to the second light emitting layer 433.

The first light emitting layer 431 and the second light emitting layer 433 may be formed of the same material or different materials. The first light emitting layer 431 may emit light having a first color, and the second light emitting layer 433 may emit light having a second color. The first color and the second color may be the same or different from each other.

The first layer 432 may include a compound 432a represented by chemical formula 1. The compound 432a is described below in detail. As the first layer 432 includes the compound 432a represented by chemical formula 1 described above, the organic light emitting element may have high efficiency, long lifespan, and/or low driving voltage.

The first layer 432 may be a charge generation layer. For example, the organic light emitting element 400 may include a charge generation layer positioned between the first light emitting layer 431 and the second light emitting layer 433. The charge generation layer may include a p-type charge generation layer and an n-type charge generation layer. In this example, the first layer 432 may be a p-type charge generation layer.

The first layer 432 may include the compound 432a represented by chemical formula 1 described above as a dopant. The above-described compound 432a may be included in the first layer 432 as a p dopant. For example, the first layer 432 may be formed by doping with 1 wt % to 40 wt % of the compound 432a represented by chemical formula 1 described above. Alternatively, the first layer 432 may essentially consist of the compound 432a represented by chemical formula 1.

The first stack may further include a functional layer in addition to the first light emitting layer 431. For example, the first stack may include a hole injection layer, a first hole transport layer, a first light emitting layer 431 and a first electron transport layer.

The second stack may further include a functional layer in addition to the second light emitting layer 433. For example, the second stack may include a second hole transport layer, a second light emitting layer 433, a second electron transport layer, and an electron injection layer.

The hole injection layer may be positioned on the first electrode 410 as the anode electrode. The first hole transport layer may be positioned on the hole injection layer. The first light emitting layer 431 may be positioned on the first hole transport layer. The first electron transport layer may be positioned on the first light emitting layer 431. The n-type charge generation layer may be positioned on the first electron transport layer. The p-type charge generation layer may be positioned on the n-type charge generation layer. The second hole transport layer may be positioned on the p-type charge generation layer. The second light emitting layer 433 may be positioned on the second hole transport layer. The second electron transport layer may be positioned on the second light emitting layer 433. The electron injection layer may be positioned on the second electron transport layer. In this example, the first layer 432 may be a p-type charge generation layer.

The hole injection layer may include an amine-based compound. For example, the hole injection layer may include one or more of HATCN (1,4,5,8,9,11-hexaazatriphenylenehexacarbonitile) and NPD (N,N′-di(1-naphthyl)-N,N′-diphenyl-(1,1′-biphenyl)-4,4′-diamine). However, the material for the hole injection layer is not limited to those described above, and may include other compounds that may be used as hole injection materials in the field of organic light emitting elements.

The first hole transport layer may include an amine-based compound. For example, the hole transport layer may include one or more of HATCN (1,4,5,8,9,11-hexaazatriphenylenehexacarbonitile) and NPD (N,N′-di(1-naphthyl)-N,N′-diphenyl-(1,1′-biphenyl)-4,4′-diamine). However, the material for the first hole transport layer is not limited to those described above, and may include other compounds that may be used as hole transport materials in the field of organic light emitting elements.

The first light emitting layer may be a fluorescent light emitting layer or a phosphorescent light emitting layer. The fluorescent light emitting layer may include one or more of a boron-based compound, an anthracene-based compound, and a pyrene-based compound. The phosphorescent emitting layer may include at least one of a carbazole-based compound and an iridium-based compound.

The first electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1-H-benzimidazole)). However, the material for the electron transport layer is not limited to those described above, and may include other compounds that may be used as electron transport materials in the field of organic light emitting elements.

The n-type charge generation layer may include a phenanthroline-based compound. The phenanthroline-based compound may be bphen (bathophenanthroline). However, the material for the n-type charge generation layer is not limited to those described above, and may include other compounds that may be used as n-type charge generation layer materials in the field of organic light emitting elements.

The p-type charge generation layer may include the compound 432a represented by chemical formula 1 described above. Further, the p-type charge generation layer may further include an amine-based compound. The amine-based compound may be NPD (N,N′-di(1-naphthyl)-N,N′-diphenyl-(1,1′-biphenyl)-4,4′-diamine). However, the amine-based compound that may be used as a material for the p-type charge generation layer is not limited to those described above. As the p-type charge generation layer includes the compound 432a represented by chemical formula 1 described above, the organic light emitting element may have high efficiency, long lifespan, and/or low driving voltage.

The second hole transport layer may include an amine-based compound. For example, the hole transport layer may include one or more of HATCN (1,4,5,8,9,11-hexaazatriphenylenehexacarbonitile) and NPD (N,N′-di(1-naphthyl)-N,N′-diphenyl-(1,1′-biphenyl)-4,4′-diamine). However, the material for the second hole transport layer is not limited to those described above, and may include other compounds that may be used as hole transport materials in the field of organic light emitting elements.

The second light emitting layer may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis (N-carbazolyl)-1,1′-biphenyl). The iridium-based compound may be Ir(ppy)₃(tris(2-phenylpyridine)iridium(III)). However, the material for the light emitting layer is not limited to those described above, and may include other compounds that may be used as light emitting layer materials in the field of organic light emitting elements.

The second electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1-H-benzimidazole)). However, the material for the electron transport layer is not limited to those described above, and may include other compounds that may be used as electron transport materials in the field of organic light emitting elements.

The electron injection layer may include at least one of an azine-based compound and an imidazole-based compound. For example, the electron injection layer may include one or more of LiF and LiQ. However, the material for the electron injection layer is not limited to those described above, and may include other compounds that may be used as electron injection materials in the field of organic light emitting elements.

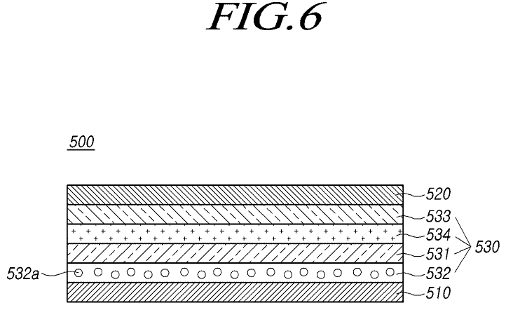

FIG. 6 is a cross-sectional view schematically illustrating an organic light emitting element 500 according to embodiments of the disclosure.

Referring to FIG. 6, the organic light emitting element 500 may include a first electrode 510, a second electrode 520, and an organic material layer 530 positioned between the first electrode 510 and the second electrode 520.

The organic material layer 530 may include a plurality of light emitting layers. For example, the organic material layer 530 may be a tandem type organic light emitting element including a first light emitting layer 531 and a second light emitting layer 533. The tandem type organic light emitting element may include a plurality of stacks each including a light emitting layer. For example, the tandem type organic light emitting element may include a first stack including a first light emitting layer 531 and a second stack including a second light emitting layer 533. In this example, the first stack may include additional functional layers in addition to the first light emitting layer 431. Further, the second stack may include additional functional layers in addition to the second light emitting layer 433.

The first light emitting layer 531 and the second light emitting layer 533 may be formed of the same material or different materials. The first light emitting layer 531 may emit light having a first color, and the second light emitting layer 533 may emit light having a second color. The first color and the second color may be the same or different from each other.

The first light emitting layer 531 may be positioned on the first electrode 510, the second light emitting layer 533 may be positioned on the first light emitting layer 531, and the second electrode 520 may be positioned on the second light emitting layer 533.

The organic material layer 530 may include a charge generation layer 534. The charge generation layer 534 may be positioned between any two light emitting layers included in the organic material layer 530. For example, the charge generation layer 534 may be positioned between the first light emitting layer 531 and the second light emitting layer 533.

The organic material layer 530 may include a first layer 532. The first layer 532 may be positioned between, e.g., the first electrode 510 and the first light emitting layer 531. In this example, the first electrode 510 may be an anode electrode.

The first layer 532 may include a compound 532a represented by chemical formula 1 described above. The compound 532a is described below in detail. When the first layer 532 including the compound 532a is positioned between the first electrode 510 and the first light emitting layer 531, the organic light emitting element 500 may have high efficiency, long lifespan and/or low driving voltage.

The first layer 532 may be a hole injection layer or a hole transport layer. For example, the first layer 532 may be a hole injection layer. When the first layer 532 including the compound 532a represented by chemical formula 1 described above is a hole injection layer or a hole transport layer, the organic light emitting element 500 may have high efficiency, long lifespan, or low driving voltage.

The first layer 532 may include the compound 532a represented by chemical formula 1 described above as a dopant. For example, the first layer 532 may be formed by doping with 1 wt % to 40 wt % of the compound 532a represented by chemical formula 1 described above. Alternatively, the first layer 532 may essentially consist of the compound 532a represented by chemical formula 1.

The first stack may further include a functional layer in addition to the first light emitting layer 531. For example, the first stack may include a hole injection layer, a first hole transport layer, a first light emitting layer 531 and a first electron transport layer.

The second stack may further include a functional layer in addition to the second light emitting layer 533. For example, the second stack may include a second hole transport layer, a second light emitting layer 533, a second electron transport layer, and an electron injection layer.

The hole injection layer may be positioned on the first electrode 510 as the anode electrode. The first hole transport layer may be positioned on the hole injection layer. The first light emitting layer 531 may be positioned on the first hole transport layer. The first electron transport layer may be positioned on the first light emitting layer 531. The charge generation layer 534 may include an n-type charge generation layer and a p-type charge generation layer. The n-type charge generation layer may be positioned on the first electron transport layer. The p-type charge generation layer may be positioned on the n-type charge generation layer. The second hole transport layer may be positioned on the p-type charge generation layer. The second light emitting layer 532 may be positioned on the second hole transport layer. The second electron transport layer may be positioned on the second light emitting layer 532. The electron injection layer may be positioned on the second electron transport layer. In this example, the first layer 532 may be a hole injection layer or a first hole transport layer.

Matters regarding the hole injection layer, the first hole transport layer, the first light emitting layer 531, the first electron transport layer, the charge generation layer, the second hole transport layer, the second light emitting layer 533, the second electron transport layer, and the electron injection layer of the organic light emitting device 500 illustrated in FIG. 6 may be the same as those regarding the hole injection layer, the first hole transport layer, the first light emitting layer 431, the first electron transport layer, the charge generation layer 432, the second hole transport layer, the second light emitting layer 433, the second electron transport layer, and the electron injection layer described above with reference to FIG. 5, unless otherwise described.

The hole injection layer or the first hole transport layer may include the compound 532a represented by chemical formula 1 described above. As the hole injection layer or the first hole transport layer includes the compound 532a represented by chemical formula 1 described above, the organic light emitting element may have high efficiency, long lifespan, and/or low driving voltage.

The p-type charge generation layer may include an amine-based compound. The amine-based compound may be NPD (N,N′-di(1-naphthyl)-N,N′-diphenyl-(1,1′-biphenyl)-4,4′-diamine). However, the amine-based compound that may be used as a material for the p-type charge generation layer is not limited to those described above.

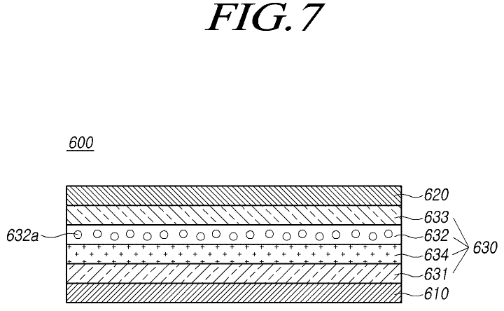

FIG. 7 is a cross-sectional view schematically illustrating an organic light emitting element 600 according to embodiments of the disclosure.

Referring to FIG. 7, the organic light emitting element 600 may include a first electrode 610, a second electrode 620, and an organic material layer 630 positioned between the first electrode 610 and the second electrode 620.

The organic material layer 630 may include a plurality of light emitting layers. For example, the organic material layer 630 may be a tandem type organic light emitting element including a first light emitting layer 631 and a second light emitting layer 633. The tandem type organic light emitting element may include a plurality of stacks each including a light emitting layer. For example, the tandem type organic light emitting element may include a first stack including a first light emitting layer 631 and a second stack including a second light emitting layer 633. In this example, the first stack may include additional functional layers in addition to the first light emitting layer 431. Further, the second stack may include additional functional layers in addition to the second light emitting layer 433.

The first light emitting layer 631 and the second light emitting layer 633 may be formed of the same material or of different materials. The first light emitting layer 631 may emit light having a first color, and the second light emitting layer 633 may emit light having a second color. The first color and the second color may be the same or different from each other.

The first light emitting layer 631 may be positioned on the first electrode 610, the second light emitting layer 633 may be positioned on the first light emitting layer 631, and the second electrode 620 may be positioned on the second light emitting layer 633.

The organic material layer 630 may include a charge generation layer 634. The charge generation layer 634 may be positioned between any two light emitting layers included in the organic material layer 630. For example, the charge generation layer 634 may be positioned between the first light emitting layer 631 and the second light emitting layer 633.

The organic material layer 630 may include a first layer 632. The first layer 632 may be positioned between, e.g., the first electrode 610 and the second light emitting layer 633. In this example, the first electrode 610 may be an anode electrode.

The first layer 632 may include a compound 632a represented by chemical formula 1 described above. The compound 632a is described below in detail. When the first layer 632 including the compound 632a is positioned between the first electrode 610 and the first light emitting layer 631, the organic light emitting element 600 may have high efficiency, long lifespan and/or low driving voltage.

The first layer 632 may be a hole injection layer or a hole transport layer. For example, the first layer 632 may be a hole injection layer. When the first layer 632 including the compound 632a represented by chemical formula 1 described above is a hole injection layer or a hole transport layer, the organic light emitting element 600 may have high efficiency, long lifespan, and/or low driving voltage.

The first layer 632 may include the compound 632a represented by chemical formula 1 described above as a dopant. The above-described compound 632a may be included in the first layer 632 as a p dopant. For example, the first layer 632 may be formed by doping with 1 wt % to 40 wt % of the compound 632a represented by chemical formula 1 described above. Alternatively, the first layer 632 may essentially consist of the compound 632a represented by chemical formula 1.

The first stack may further include a functional layer in addition to the first light emitting layer 631. For example, the first stack may include a hole injection layer, a first hole transport layer, a first light emitting layer 631 and a first electron transport layer.

The second stack may further include a functional layer in addition to the second light emitting layer 633. For example, the second stack may include a second hole transport layer, a second light emitting layer 633, a second electron transport layer, and an electron injection layer.

A hole injection layer may be positioned on the first electrode 610 as the anode electrode. A first hole transport layer may be positioned on the hole injection layer. The first light emitting layer 631 may be positioned on the first hole transport layer. A first electron transport layer may be positioned on the first light emitting layer 631. The charge generation layer 634 may include an n-type charge generation layer and a p-type charge generation layer. The n-type charge generation layer may be positioned on the first electron transport layer. The p-type charge generation layer may be positioned on the n-type charge generation layer. The first layer 632 may be positioned on the p-type charge generation layer as a second hole transport layer. The second light emitting layer 633 may be positioned on the second hole transport layer. A second electron transport layer may be positioned on the second light emitting layer 633. An electron injection layer may be positioned on the second electron transport layer. In this example, the first layer 632 is a hole transport layer.

Matters regarding the hole injection layer, the first hole transport layer, the first light emitting layer 631, the first electron transport layer, the charge generation layer 634, the second hole transport layer, the second light emitting layer 633, the second electron transport layer, and the electron injection layer of the organic light emitting device 600 illustrated in FIG. 7 may be the same as those regarding the hole injection layer, the first hole transport layer, the first light emitting layer 431, the first electron transport layer, the charge generation layer 432, the second hole transport layer, the second light emitting layer 433, the second electron transport layer, and the electron injection layer described above with reference to FIG. 5, unless otherwise described.

The p-type charge generation layer may include an amine-based compound. The amine-based compound may be NPD (N,N′-di(1-naphthyl)-N,N′-diphenyl-(1,1′-biphenyl)-4,4′-diamine). However, the amine-based compound that may be used as a material for the p-type charge generation layer is not limited to those described above.

The second hole transport layer may include the compound represented by chemical formula 2 described above. As the second hole transport layer includes the compound represented by chemical formula 1 described above, the organic light emitting element may have high efficiency, long lifespan, and/or low driving voltage.

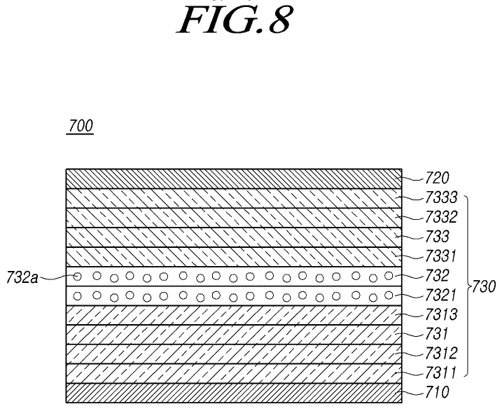

FIG. 8 is a view schematically illustrating an organic light emitting element according to embodiments of the disclosure. Matters regarding the organic light emitting element illustrated in FIG. 8 may be the same as those described for the organic light emitting element shown in FIG. 5 unless otherwise described.

The organic light emitting element 700 may include a first electrode 710, a second electrode 720, and an organic material layer 730 positioned between the first electrode 710 and the second electrode 720.

The organic material layer 730 may include a first light emitting layer 731, a second light emitting layer 733, and a first layer 732 positioned between the first light emitting layer 731 and the second light emitting layer 733. In other words, the organic light emitting element 700 may be a tandem type organic light emitting element including two or more light emitting layers. The tandem type organic light emitting element may include a plurality of stacks each including a light emitting layer. For example, the tandem type organic light emitting element may include a first stack including a first light emitting layer 731 and a second stack including a second light emitting layer 733.

The first stack may further include additional functional layers in addition to the first light emitting layer 731. For example, the first stack may include a hole injection layer 7311 positioned on the first electrode 710, a first hole transport layer 7312 positioned on the hole injection layer 7311, a first light emitting layer 731 positioned on the first hole transport layer 7312, and a first electron transport layer 7313 positioned on the first light emitting layer 731.

Further, the second stack may include additional functional layers in addition to the second light emitting layer 733. For example, the second stack may include a second hole transport layer 7331, a second light emitting layer 733 positioned on the second hole transport layer 7331, a second electron transport layer 7332 positioned on the second light emitting layer 733, and an electron injection layer 7333 positioned on the second electron transport layer 7332.

The organic light emitting element 700 may include a charge generation layer positioned between the first stack and the second stack. The charge generation layer may include an n-type charge generation layer 7321 and a first layer 732. In this example, the first layer 732 may be a p-type charge generation layer.

The first layer 732 may include compound 732a represented by chemical formula 1 as a p dopant. The compound 732a represented by chemical formula 1 is described below in detail.

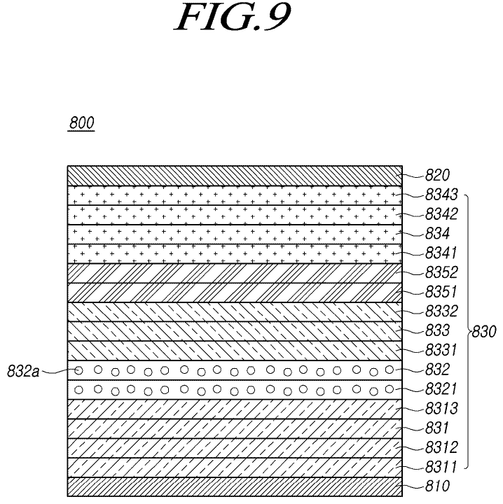

FIG. 9 is a view schematically illustrating an organic light emitting element according to embodiments of the disclosure. Matters regarding the organic light emitting element illustrated in FIG. 9 may be the same as those described for the organic light emitting element shown in FIG. 8 unless otherwise described.

The organic light emitting element 800 may include a first electrode 810, a second electrode 820, and an organic material layer 830 positioned between the first electrode 810 and the second electrode 820.

The organic material layer 830 may include a first light emitting layer 831, a second light emitting layer 833, a third light emitting layer 834, and a first layer 832 positioned between the first light emitting layer 831 and the second light emitting layer 833. In other words, the organic light emitting element 800 may be a tandem type organic light emitting element including three or more light emitting layers. The tandem type organic light emitting element may include a plurality of stacks each including a light emitting layer. For example, the tandem type organic light emitting element may include a first stack including a first light emitting layer 831, a second stack including a second light emitting layer 833, and a third stack including a third light emitting layer 834.

The first stack may further include additional functional layers in addition to the first light emitting layer 831. For example, the first stack may include a hole injection layer 8311 positioned on the first electrode 810, a first hole transport layer 8312 positioned on the hole injection layer 8311, a first light emitting layer 831 positioned on the first hole transport layer 8312, and a first electron transport layer 8313 positioned on the first light emitting layer 831.

Further, the second stack may include additional functional layers in addition to the second light emitting layer 833. For example, the second stack may include a second hole transport layer 8331, a second light emitting layer 833 positioned on the second hole transport layer 8331, and a second electron transport layer 8332 positioned on the second light emitting layer 833.

Further, the third stack may include additional functional layers in addition to the third light emitting layer 834. For example, the third stack may include a third hole transport layer 8341, a third light emitting layer 834 positioned on the third hole transport layer 8341, a third electron transport layer 8342 positioned on the third light emitting layer 834, and an electron injection layer 8343 positioned on the third electron transport layer 8342.

The organic light emitting element 800 may include a first charge generation layer positioned between the first stack and the second stack. The first charge generation layer may include a first n-type charge generation layer 8321 and a first layer 832. In this example, the first layer 832 may be a p-type charge generation layer.

The first layer 832 may include a compound 832a represented by chemical formula 1 as a p dopant. The compound 832a represented by chemical formula 1 is described below in detail.

The organic light emitting element 800 may include a second charge generation layer positioned between the second stack and the third stack. The second charge generation layer may include a second n-type charge generation layer 8351 and a p-type charge generation layer 8352. The p-type charge generation layer 8352 may include a p dopant 832b. The p dopant 832b may be the same as the compound 832a represented by chemical formula 1 of the first layer 832.

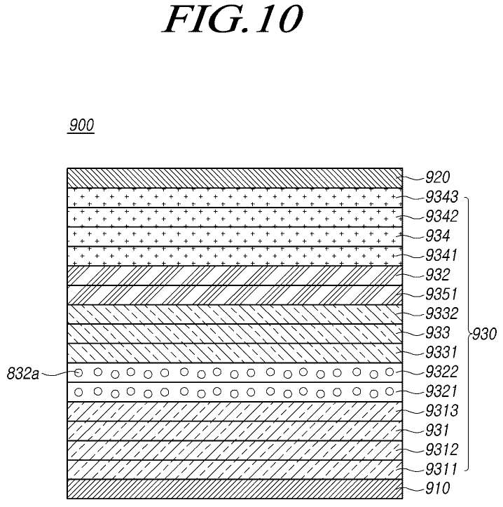

FIG. 10 is a view schematically illustrating an organic light emitting element according to embodiments of the disclosure. Matters regarding the organic light emitting element illustrated in FIG. 10 may be the same as those described for the organic light emitting element shown in FIG. 9 unless otherwise described.

The organic light emitting element 900 may include a first electrode 910, a second electrode 920, and an organic material layer 930 positioned between the first electrode 910 and the second electrode 920.

The organic material layer 930 may include a first light emitting layer 931, a second light emitting layer 933, a third light emitting layer 934, and a first layer 932 positioned between the second light emitting layer 933 and the third light emitting layer 934. In other words, the organic light emitting element 900 may be a tandem type organic light emitting element including three or more light emitting layers. The tandem type organic light emitting element may include a plurality of stacks each including a light emitting layer. For example, the tandem type organic light emitting element may include a first stack including a first light emitting layer 931, a second stack including a second light emitting layer 933, and a third stack including a third light emitting layer 934.

The first stack may further include additional functional layers in addition to the first light emitting layer 931. For example, the first stack may include a hole injection layer 9311 positioned on the first electrode 910, a first hole transport layer 9312 positioned on the hole injection layer 9311, a first light emitting layer 931 positioned on the first hole transport layer 9312, and a first electron transport layer 9313 positioned on the first light emitting layer 931.

Further, the second stack may include additional functional layers in addition to the second light emitting layer 933. For example, the second stack may include a second hole transport layer 9331, a second light emitting layer 933 positioned on the second hole transport layer 9331, and a second electron transport layer 9332 positioned on the second light emitting layer 933.

Further, the third stack may include additional functional layers in addition to the third light emitting layer 934. For example, the third stack may include a third hole transport layer 9341, a third light emitting layer 934 positioned on the third hole transport layer 9341, a third electron transport layer 9342 positioned on the third light emitting layer 934, and an electron injection layer 9343 positioned on the third electron transport layer 9342.

The organic light emitting element 900 may include a first charge generation layer positioned between the first stack and the second stack. The first charge generation layer may include a first n-type charge generation layer 9321 and a p-type charge generation layer 9322.

The organic light emitting element 900 may include a second charge generation layer positioned between the second stack and the third stack. The second charge generation layer may include a second n-type charge generation layer 9351 and a first layer 932. The first layer 932 may be a p-type charge generation layer. The first layer 932 may include a compound 932a represented by chemical formula 1 as a p dopant. The compound 932a represented by chemical formula 1 is described below in detail.

The p-type charge generation layer 9322 may include a p dopant 932b. The p dopant 932b may be the same as the compound 932a represented by chemical formula 1.

The compounds 232a, 332a, 432a, 532a, 632a, 732a, 832a, and 932a represented by chemical formula 1 described above are described below.

The above-described compounds 232a, 332a, 432a, 532a, 632a, 732a, 832a, and 932a are represented by chemical formula 1 below.

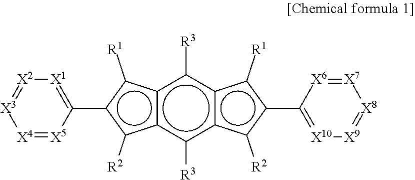

Hereinafter, chemical formula 1 is described.

R¹ and R² are each independently selected from the group consisting of hydrogen, deuterium, tritium, halogen, a cyano group, an alkyl group of C₁-C₅₀, a haloalkyl group of C₁-C₅₀, an alkoxy group of C₁-C₃₀, a haloalkoxy group of C₁-C₃₀, an aryl group of C₆-C₆₀, a haloaryl group of C₆-C₆₀, a heterocyclic group of C₂-C₆₀ including at least one heteroatom among O, N, S, Si and P, a haloheterocyclic group of C₂-C₆₀ including at least one heteroatom among O, N, S, Si and P, and malononitrile.

For example, R¹ and R² may be each independently selected from the group consisting of hydrogen, deuterium, tritium, and a cyano group.

One R³ is a halogen or a cyano group, and the other R³ is selected from the group consisting of hydrogen, deuterium, tritium, a haloalkyl group of C₁-C₅₀, a haloalkoxy group of C₁-C₃₀, and a haloaryl group of C₆-C₆₀ substituted with one or more of a halogen and a haloalkoxy group of C₁-C₃₀. In other words, one R³ is a halogen or cyano group, which is an electron withdrawing group (EWG), and the other R³ is a substituent having an increased steric demand/volume. As the compounds 232a, 332a, 432a, 532a, 632a, 732a, 832a, and 932a have such a structure, the organic light emitting elements 200, 300, 400, 500, 600, 700, 800, and 900 may have excellent efficiency, a long lifespan, and/or a low driving voltage.

X¹ to X⁵ are each independently CR^{a} or N, and at least two of X¹ to X⁵ are CR^{a}.

R^{a} is each independently selected from the group consisting of hydrogen, deuterium, tritium, halogen, a cyano group, an alkyl group of C₁-C₅₀, a haloalkyl group of C₁-C₅₀, an alkoxy group of C₁-C₅₀, and a haloalkoxy group of C₁-C₅₀. At least one of R^{a} is selected from the group consisting of halogen, a cyano group, a haloalkyl group of C₁-C₅₀, and a haloalkoxy group of C₁-C₅₀. In other words, at least one of R^{a} is an electron withdrawing group (EWG).

For example, R^{a} may be each independently selected from among hydrogen, deuterium, tritium, halogen, a cyano group, a haloalkyl group of C₁-C₅₀, and a haloalkoxy group of C₁-C₅₀. At least one of R^{a} may be selected from the group consisting of halogen, a cyano group, a haloalkyl group of C₁-C₅₀, and a haloalkoxy group of C₁-C₅₀.

When R^{a} is a haloalkyl group, R^{a} may be a haloalkyl group of C₁-C₃₀, a haloalkyl group of C₁-C₁₅, a haloalkyl group of C₁-C₁₀, or a haloalkyl group of C₁-C₃.

When R^{a} is a haloalkoxy group, R^{a} may be a haloalkoxy group of C₁-C₃₀, a haloalkoxy group of C₁-C₁₅, a haloalkoxy group of C₁-C₁₀ or a haloalkoxy group of C₁-C₃.

X⁶ to X¹⁰ are each independently CR^{b} or N, and at least two of X⁶ to X¹⁰ are CR^{b}.

R^{b} is each independently selected from the group consisting of hydrogen, deuterium, tritium, halogen, a cyano group, an alkyl group of C₁-C₅₀, a haloalkyl group of C₁-C₅₀, an alkoxy group of C₁-C₅₀, and a haloalkoxy group of C₁-C₅₀. At least one of R^{b} is selected from the group consisting of halogen, a cyano group, a haloalkyl group of C₁-C₅₀, and a haloalkoxy group of C₁-C₅₀. In other words, at least one of R^{b} is an electron withdrawing group (EWG).

For example, R^{b} may be each independently selected from among hydrogen, deuterium, tritium, halogen, a cyano group, a haloalkyl group of Cₐ-C₅₀, and a haloalkoxy group of C₁-C₅₀. At least one of R^{b} may be selected from the group consisting of halogen, a cyano group, a haloalkyl group of C₁-C₅₀, and a haloalkoxy group of C₁-C₅₀.

When R^{b} is a haloalkyl group, R^{b} may be a haloalkyl group of C₁-C₃₀, a haloalkyl group of C₁-C₁₅, a haloalkyl group of C₁-C₁₀, or a haloalkyl group of C₁-C₃.

When R^{b} is a haloalkoxy group, R^{a} may be a haloalkoxy group of C₁-C₃₀, a haloalkoxy group of C₁-C₁₅, a haloalkoxy group of C₁-C₁₀, or a haloalkoxy group of C₁-C₃.

Independently each of R¹ to R³, R^{a} and R^{b} in chemical formula 1 may be further substituted. E.g., in an alkyl group, a haloalkyl group, an alkoxy group, a haloalkoxy group, an aryl group, a haloaryl group, a heterocyclic group, and a haloheterocyclic group, at least one substituent selected from the group consisting of deuterium, a nitro group, a cyano group, an amino group, an alkoxy group of C₁-C₂₀, a haloalkoxy group of C₁-C₂₀, an alkyl group of C₁-C₂₀, a haloalkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C₆₀, an arylsilyl group of C₁₈-C₆₀, and an alkylarylsilyl group of C₈-C₆₀ may be further substituted. That is, each of the alkyl group, the haloalkyl group, the alkoxy group, the haloalkoxy group, the aryl group, the haloaryl group, the heterocyclic group, and the haloheterocyclic group may independently be further substituted with a substituent selected from the group consisting of deuterium, a nitro group, a cyano group, an amino group, an alkoxy group of C₁-C₂₀, a haloalkoxy group of C₁-C₂₀, an alkyl group of C₁-C₂₀, a haloalkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C₆₀, an arylsilyl group of C₁₈-C₆₀, and an alkylarylsilyl group of C₈-C₆₀.

The compounds 232a, 332a, 432a, 532a, 632a, 732a, 832a, and 932a represented by chemical formula 1 may be represented by any one of chemical formula 2 and chemical formula 3 below.

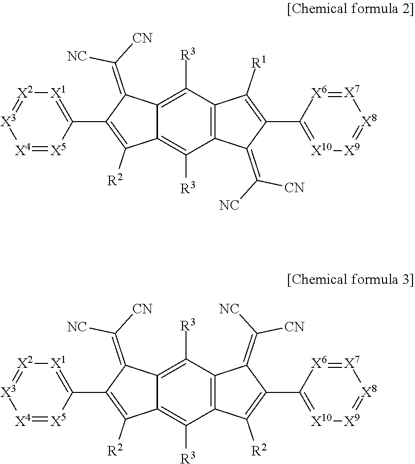

In chemical formula 2 and chemical formula 3, R¹ to R³ and X¹ to X¹⁰ may be the same as R¹ to R³ and X¹ to X¹⁰ defined in chemical formula 1 described above.

The compounds 232a, 332a, 432a, 532a, 632a, 732a, 832a, and 932a represented by chemical formula 1 may be represented by any one of chemical formula 4 and chemical formula 5 below.

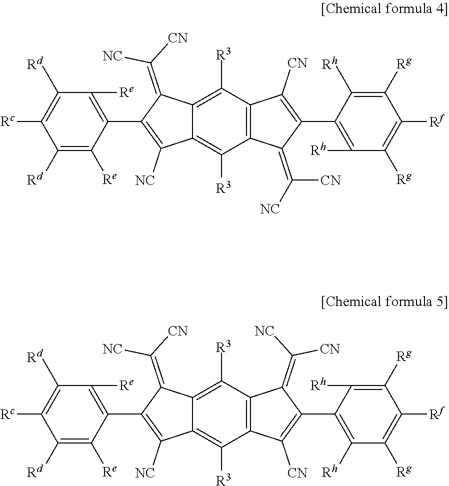

Hereinafter, chemical formula 4 and chemical formula 5 are described.

R^{c} to R^{h} are each independently selected from the group consisting of hydrogen; deuterium; tritium; halogen; an alkyl group of C₁-C₅₀; a haloalkyl group of C₁-C₅₀; an alkoxy group of C₁-C₅₀; and a haloalkoxy group of C₁-C₅₀

When R^{c} to R^{h} are a haloalkyl group, R^{c} to R^{h} which are a haloalkyl group may be a haloalkyl group of C₁-C₃₀, a haloalkyl group of C₁-C₁₅, or a haloalkyl group of C₁-C₁₀.

When R^{c} to R^{h} are a haloalkoxy group, R^{c} to R^{h} may be a haloalkoxy group of C₁-C₃₀, a haloalkoxy group of C₁-C₁₅, or a haloalkoxy group of C₁-C₁₀.

R³ is the same as R³ to R⁷ defined in chemical formula 1.

Hereinafter, chemical formula 4 is described in more detail.

R^{c} may be selected from the group consisting of halogen, a haloalkyl group of C₁-C₅₀, and a haloalkoxy group of C₁-C₅₀. In other words, R^{c} may be an electron withdrawing group (EWG).

One R^{d} may be hydrogen, deuterium, or tritium, and the other R^{d} may be selected from the group consisting of halogen, a haloalkyl group of C₁-C₅₀ and a haloalkoxy group of C₁-C₅₀. In this example, one R^{d} of the two R^{d}s may be an electron withdrawing group (EWG).

In another example, two R^{d}s may be each independently selected from the group consisting of halogen, a haloalkyl group of C₁-C₅₀, and a haloalkoxy group of C₁-C₅₀. In this example, both the R^{d}s may be an electron withdrawing group (EWG).

In the benzene ring in which R^{c} and R^{d} are substituted in chemical formula 4, R^{c}, which is in a para position in relation to the indacene moiety, may be an electron withdrawing group (EWG), and at least one of R^{c}, which is in a para position in relation to the indacene moiety, and R^{d}, which is in a meta position in relation to the indacene moiety, is an electron withdrawing group (EWG). In R^{c} and R^{d}, the electron withdrawing group (EWG) does not include a cyano group. The organic light emitting elements 200, 300, 400, 500, 600, 700, 800, and 900 including the compounds 232a, 332a, 432a, 532a, 632a, 732a, 832a, and 932a having such a structure and represented in chemical formula 4 have excellent efficiency, a long lifespan, or a low driving voltage.

R^{e} may be each independently selected from among hydrogen, deuterium, tritium, halogen, a cyano group, a haloalkyl group of C₁-C₅₀, and a haloalkoxy group of C₁-C₃₀.

R^{f} may be selected from the group consisting of halogen, a haloalkyl group of C₁-C₅₀, and a haloalkoxy group of C₁-C₅₀. In other words, R^{f} may be an electron withdrawing group (EWG).

R^{g} may be each independently selected from the group consisting of halogen, a haloalkyl group of C₁-C₅₀, and a haloalkoxy group of C₁-C₅₀. In other words, R^{g} may be an electron withdrawing group (EWG).

One R^{h} may be hydrogen, deuterium, or tritium, and the other R^{h} may be selected from the group consisting of halogen, a haloalkyl group of C₁-C₅₀ and a haloalkoxy group of C₁-C₅₀. In this example, one R^{h} of the two R^{h}s may be an electron withdrawing group (EWG).

In another example, two R^{h}s may be each independently selected from the group consisting of halogen, a haloalkyl group of C₁-C₅₀, and a haloalkoxy group of C₁-C₅₀. In this example, both the R^{h}s may be an electron withdrawing group (EWG).

In the benzene ring in which R^{f} and R^{g} are substituted in chemical formula 4, R^{f}, which is in a para position in relation to indacene moiety, may be an electron withdrawing group (EWG), and at least one of R^{f}, which is in a para position in relation to the indacene moiety, and R^{g}, which is in a meta position in relation to indacene moiety, is an electron withdrawing group (EWG). In R^{f} and R^{g}, the electron withdrawing group (EWG) does not include a cyano group. The organic light emitting elements 200, 300, 400, 500, 600, 700, 800, and 900 including the compounds 232a, 332a, 432a, 532a, 632a, 732a, 832a, and 932a having such a structure and represented in chemical formula 4 have excellent efficiency, a long lifespan, or a low driving voltage.

R^{h} may be each independently selected from among hydrogen, deuterium, tritium, halogen, a cyano group, a haloalkyl group of C₁-C₅₀, and a haloalkoxy group of C₁-C₃₀.

Hereinafter, chemical formula 5 is described in more detail.

R^{c} may be selected from the group consisting of halogen, a haloalkyl group of C₁-C₅₀, and a haloalkoxy group of C₁-C₅₀. In other words, R^{c} may be an electron withdrawing group (EWG).

One R^{d} may be hydrogen, deuterium, or tritium, and the other R^{d} may be selected from the group consisting of halogen, a haloalkyl group of C₁-C₅₀ and a haloalkoxy group of C₁-C₅₀. In this example, one R^{d} of the two R^{d}s may be an electron withdrawing group (EWG).

In another example, two R^{d}s may be each independently selected from the group consisting of halogen, a haloalkyl group of C₁-C₅₀, and a haloalkoxy group of C₁-C₅₀. In this example, both the R^{d}s may be an electron withdrawing group (EWG).

In the benzene ring in which R^{c} and R^{d} are substituted in chemical formula 5, R^{c}, which is in a para position in relation to indacene moiety, may be an electron withdrawing group (EWG), and at least one of R^{c}, which is in a para position in relation to indacene moiety, and R^{d}, which is in a meta position in relation to indacene moiety, is an electron withdrawing group (EWG). In R^{c} and R^{d}, the electron withdrawing group (EWG) does not include a cyano group. The organic light emitting elements 200, 300, 400, 500, 600, 700, 800, and 900 including the compounds 232a, 332a, 432a, 532a, 632a, 732a, 832a, and 932a having such a structure and represented in chemical formula 5 have excellent efficiency, a long lifespan, or a low driving voltage.

R^{e} may be each independently selected from among hydrogen, deuterium, tritium, halogen, a cyano group, a haloalkyl group of C₁-C₅₀, and a haloalkoxy group of C₁-C₃₀.

R^{f} may be selected from the group consisting of halogen, a haloalkyl group of C₁-C₅₀, and a haloalkoxy group of C₁-C₅₀. In other words, R^{f} may be an electron withdrawing group (EWG).

R^{g} may be each independently selected from the group consisting of halogen, a haloalkyl group of C₁-C₅₀, and a haloalkoxy group of C₁-C₅₀. In other words, R^{g} may be an electron withdrawing group (EWG).

One R^{h} may be hydrogen, deuterium, or tritium, and the other R^{h} may be selected from the group consisting of halogen, a haloalkyl group of C₁-C₅₀ and a haloalkoxy group of C₁-C₅₀. In this example, one R^{h} of the two R^{h}s may be an electron withdrawing group (EWG).

In another example, two R^{h}s may be each independently selected from the group consisting of halogen, a haloalkyl group of C₁-C₅₀, and a haloalkoxy group of C₁-C₅₀. In this example, both the R^{h}s may be an electron withdrawing group (EWG).

In the benzene ring in which R^{f} and R^{g} are substituted in chemical formula 5, R^{f}, which is in a para position in relation to indacene moiety, may be an electron withdrawing group (EWG), and at least one of R^{f}, which is in a para position in relation to indacene moiety, and R^{g}, which is in a meta position in relation to indacene moiety, is an electron withdrawing group (EWG). In R^{f} and R^{g}, the electron withdrawing group (EWG) does not include a cyano group. The organic light emitting elements 200, 300, 400, 500, 600, 700, 800, and 900 including the compounds 232a, 332a, 432a, 532a, 632a, 732a, 832a, and 932a having such a structure and represented in chemical formula 5 have excellent efficiency, a long lifespan, and/or a low driving voltage.

R^{h} may be each independently selected from among hydrogen, deuterium, tritium, halogen, a cyano group, a haloalkyl group of C₁-C₅₀, and a haloalkoxy group of C₁-C₃₀.

Independently each of R^{e} to R^{h} in chemical formulas 4 and 5 may be further substituted. E.g., in the haloalkyl group and haloalkoxy group, at least one substituent selected from the group consisting of deuterium, a nitro group, a cyano group, an amino group, an alkoxy group of C₁-C₂₀, a haloalkoxy group of C₁-C₂₀, an alkyl group of C₁-C₂₀, a haloalkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C₆₀, an arylsilyl group of C₁₈-C₆₀, and an alkylarylsilyl group of C₈-C₆₀ may be further substituted.

The compounds 232a, 332a, 432a, 532a, 632a, 732a, 832a, and 932a represented by chemical formula 1 may be represented by any one of chemical formula 6 to chemical formula 15 below. More specifically, the compound represented by chemical formula 2 described above may be represented by any one of chemical formulas 6 to 10, and the compound represented by chemical formula 3 described above may be represented by any one of chemical formulas 11 to 15.

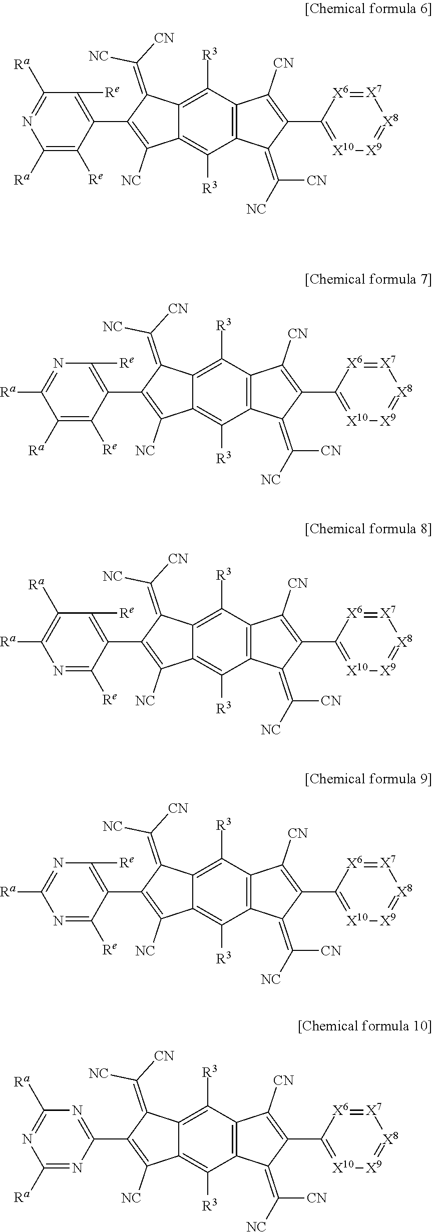

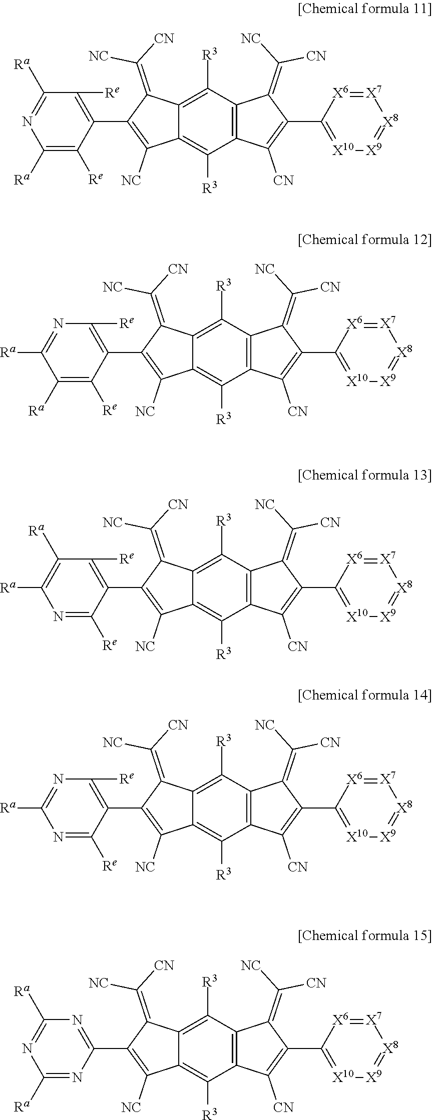

Hereinafter, chemical formulas 6 to chemical formula 15 are described.

R^{a}, R³ and X⁶ to X¹⁰ are the same as R^{a}, R³ and X⁶ to X¹⁰ defined in chemical formula 1.

R^{e} may be each independently selected from among hydrogen; deuterium; tritium; halogen; a cyano group; a haloalkyl group of C₁-C₅₀; and a haloalkoxy group of C₁-C₃₀, optionally R^{e} may be each independently selected from hydrogen, deuterium, or tritium. For example, R^{e} may be hydrogen.

The compounds 232a, 332a, 432a, 532a, 632a, 732a, 832a, and 932a represented by chemical formula 1 may be one or more of the compounds described below.

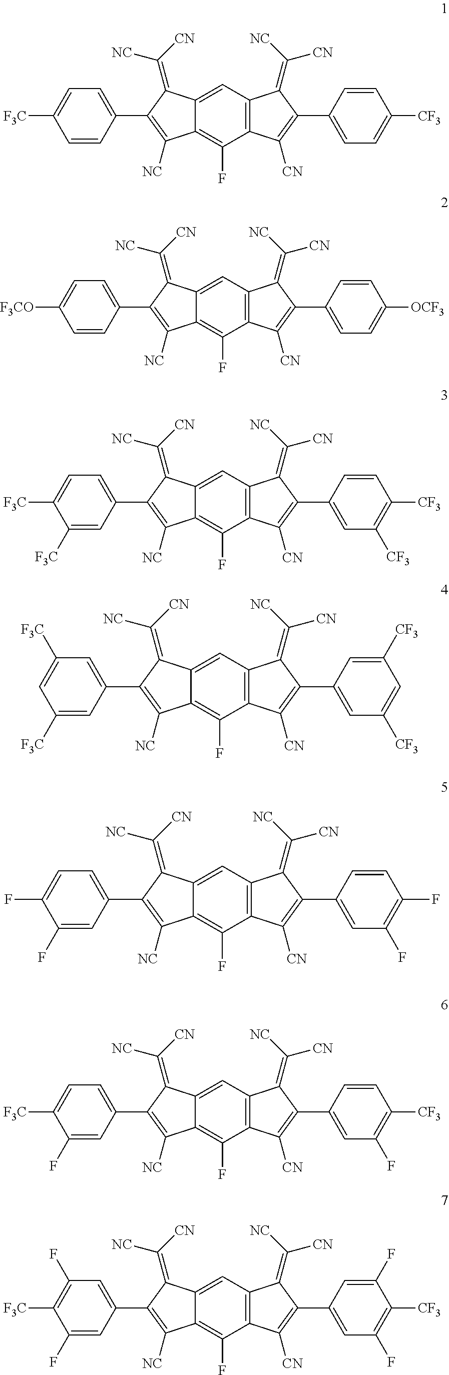

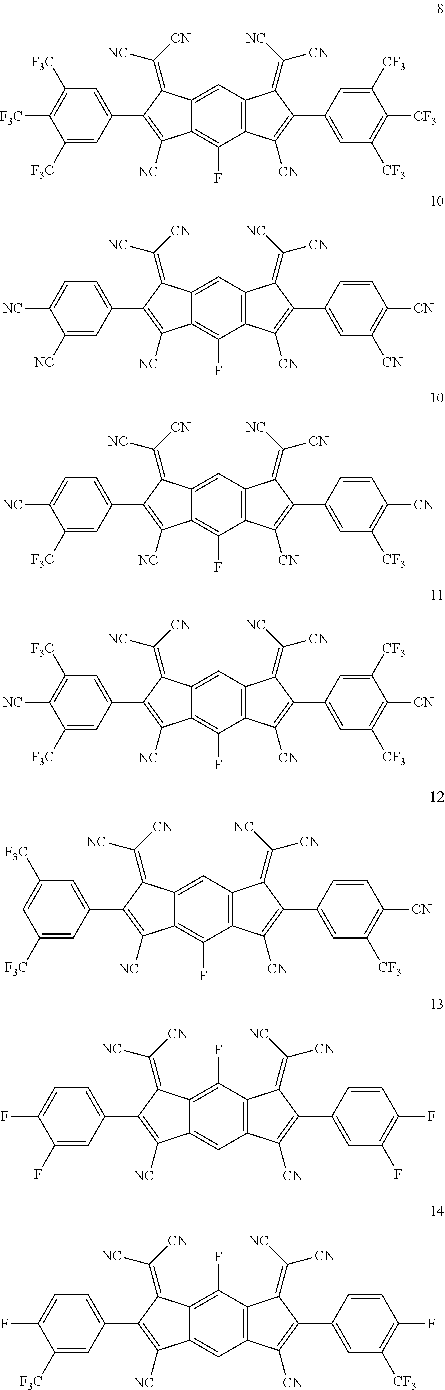

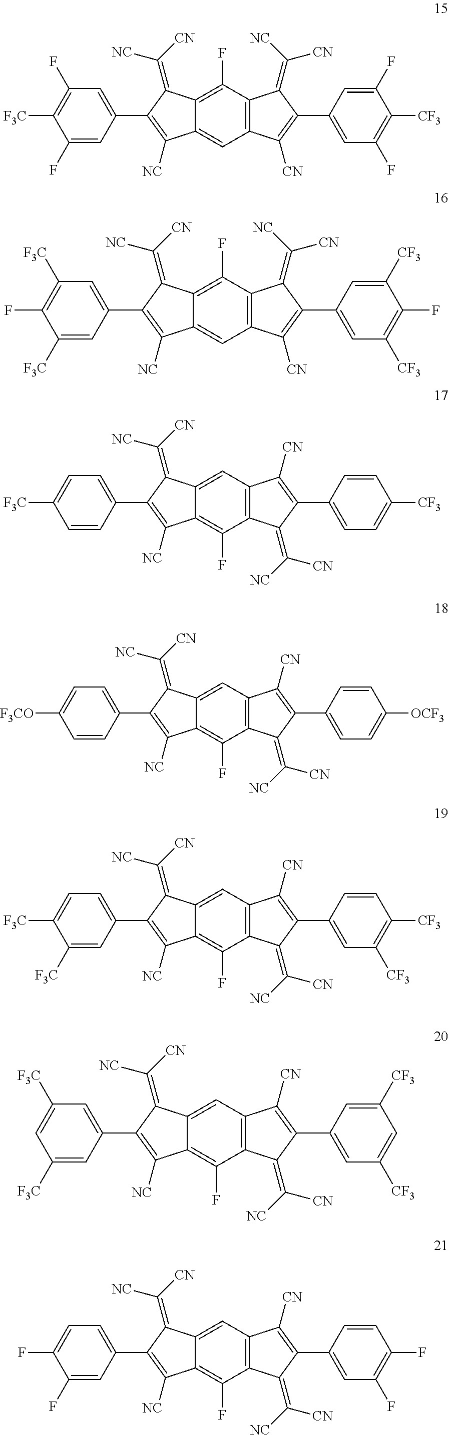

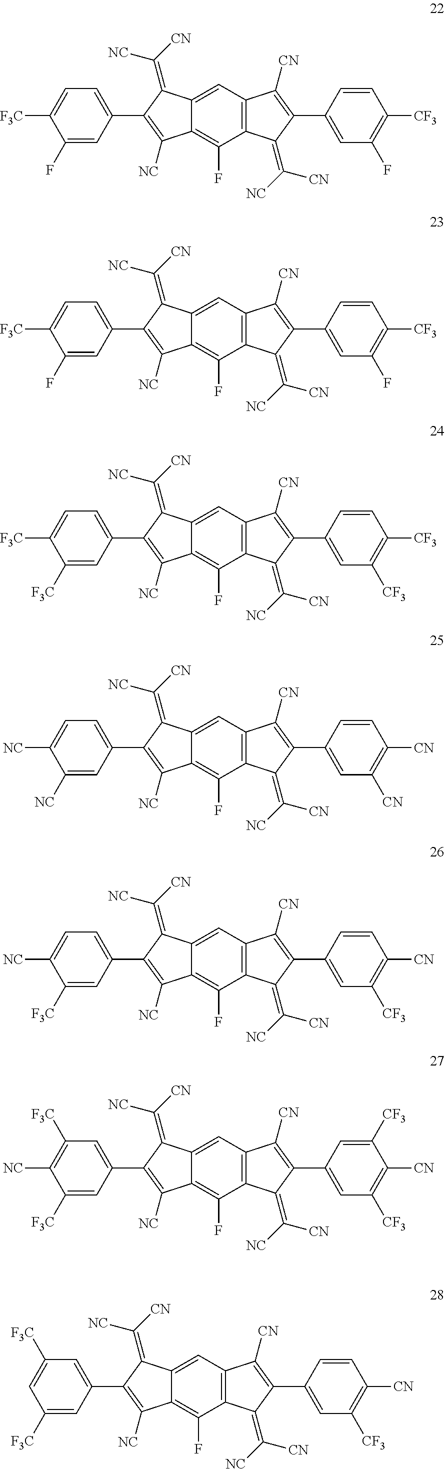

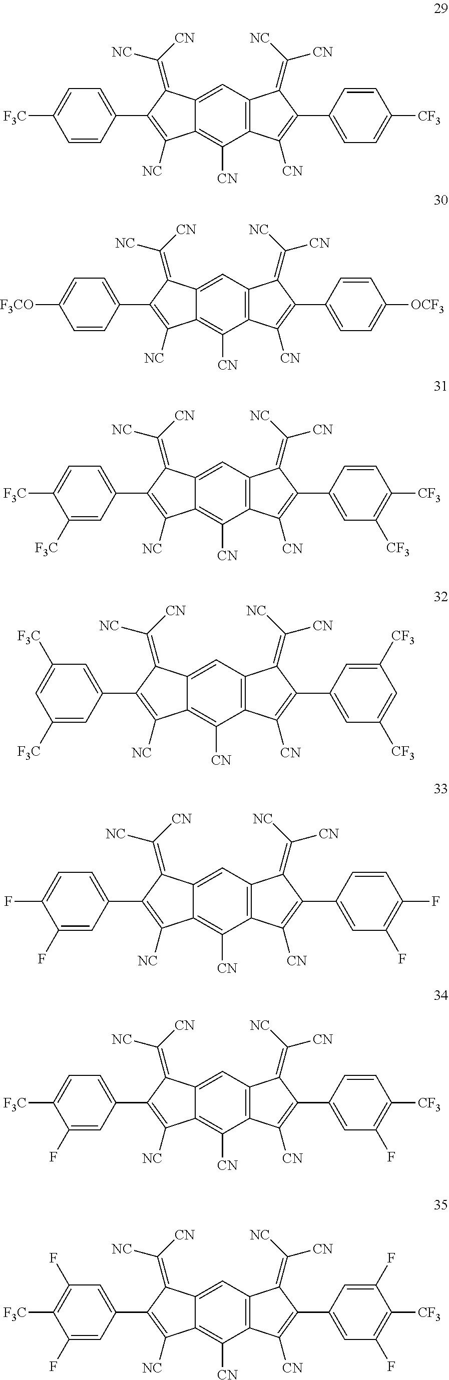

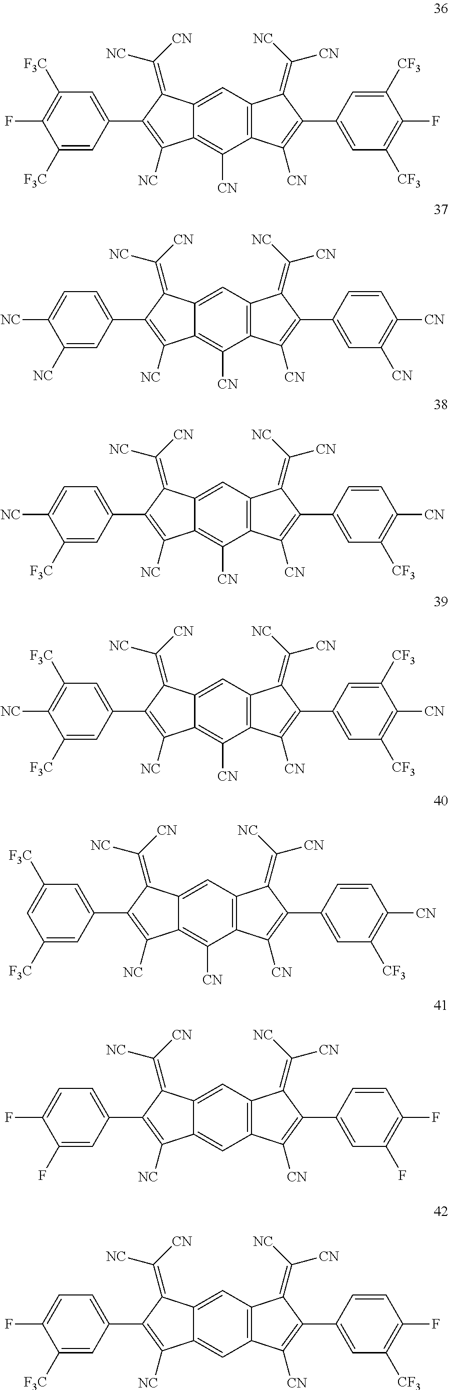

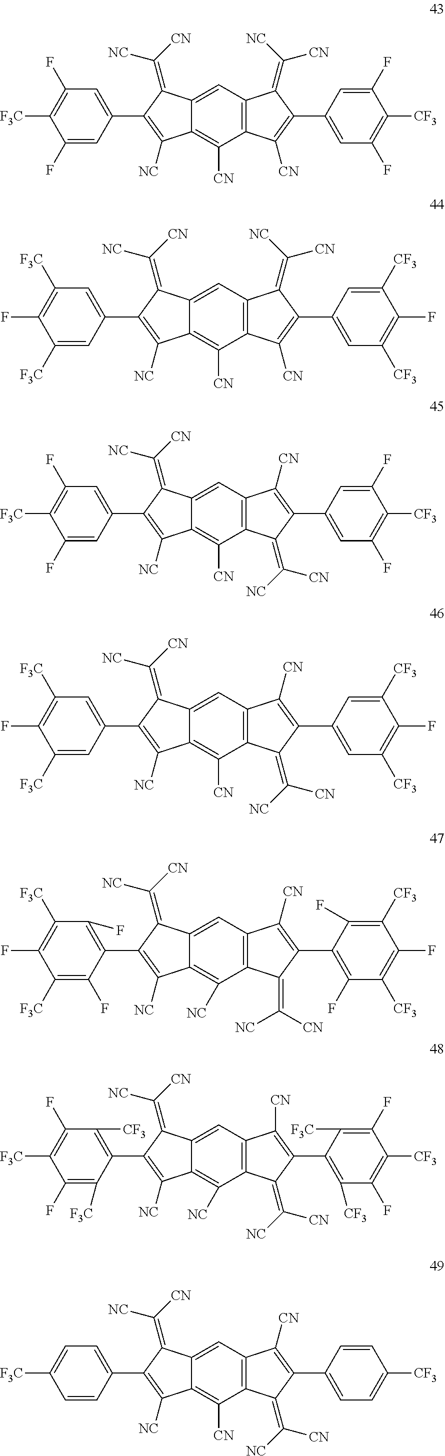

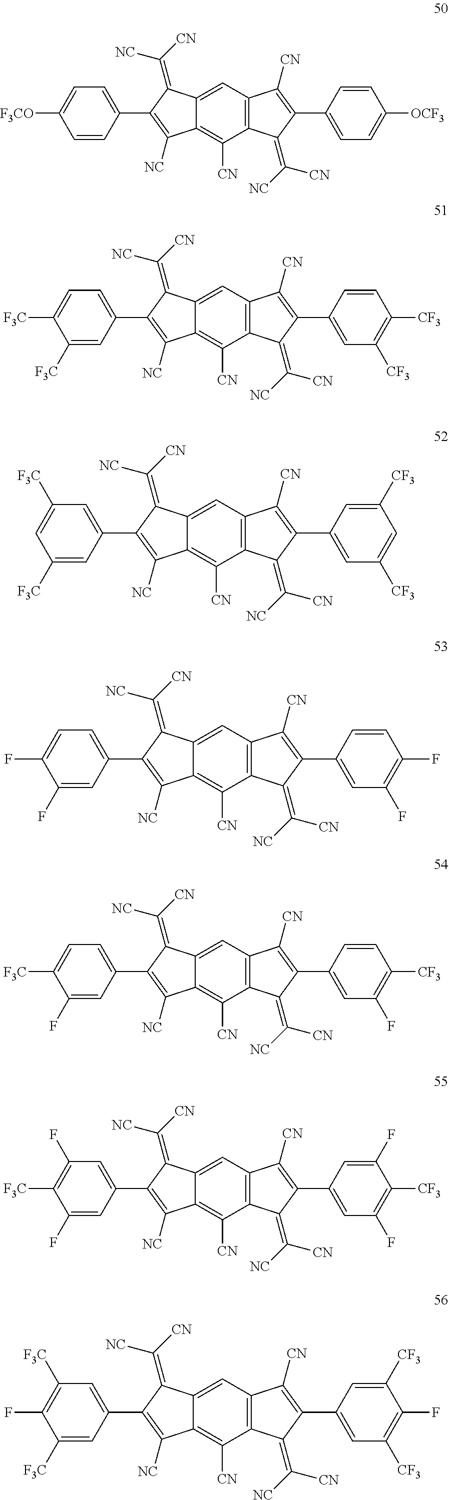

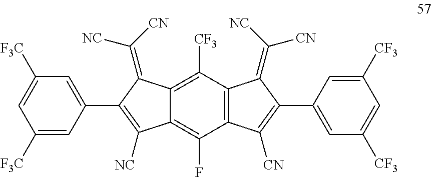 [SMILES: N#CC(C#N)=C1C(c2cc(C(F)(F)F)cc(C(F)(F)F)c2)=C(C#N)c2c(F)c3c(c(C(F)(F)F)c21)C(=C(C#N)C#N)C(c1cc(C(F)(F)F)cc(C(F)(F)F)c1)=C3C#N]

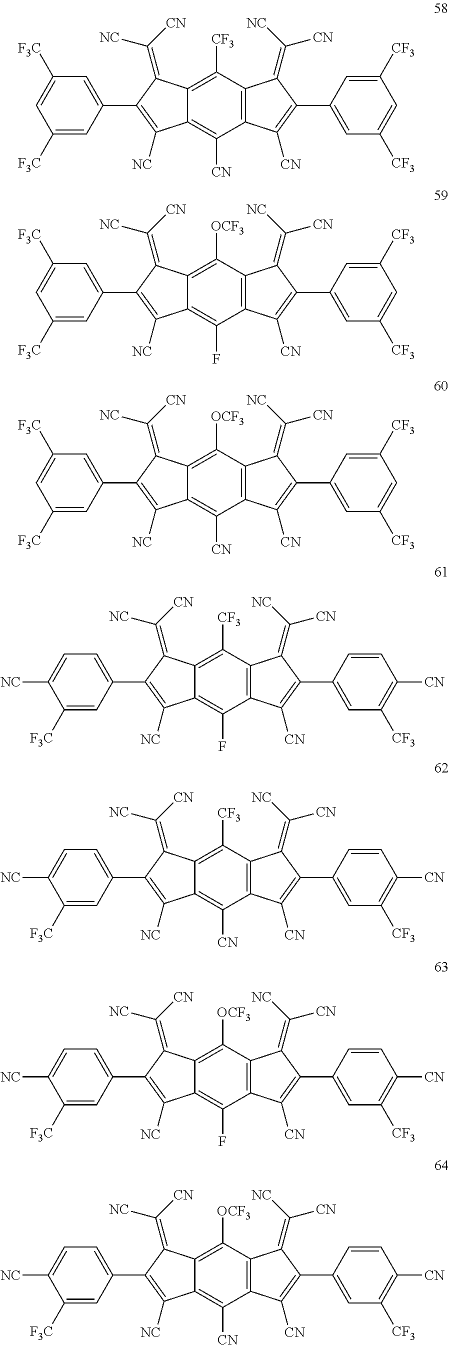

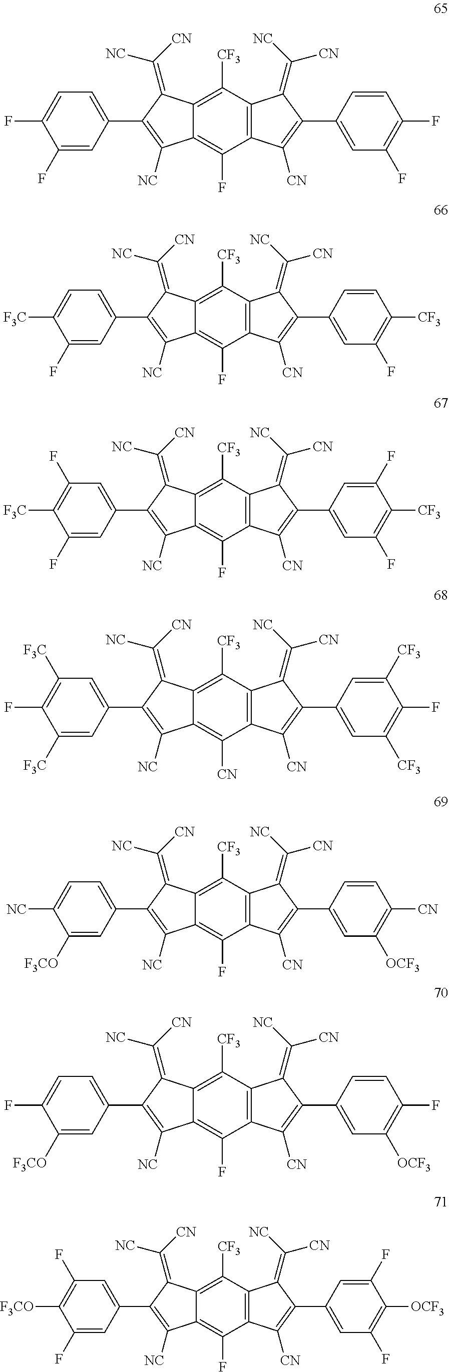

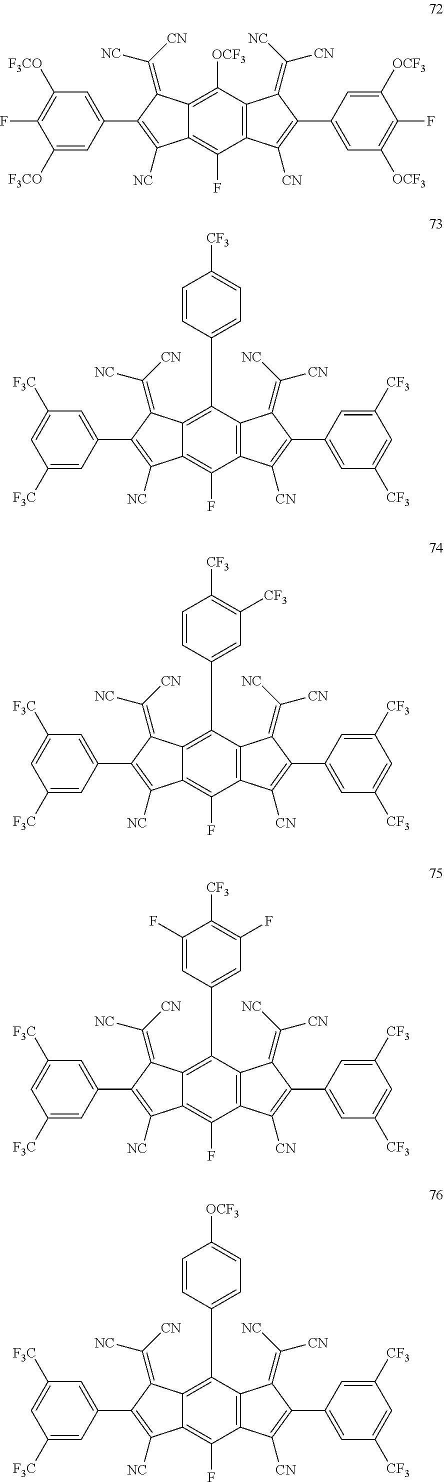

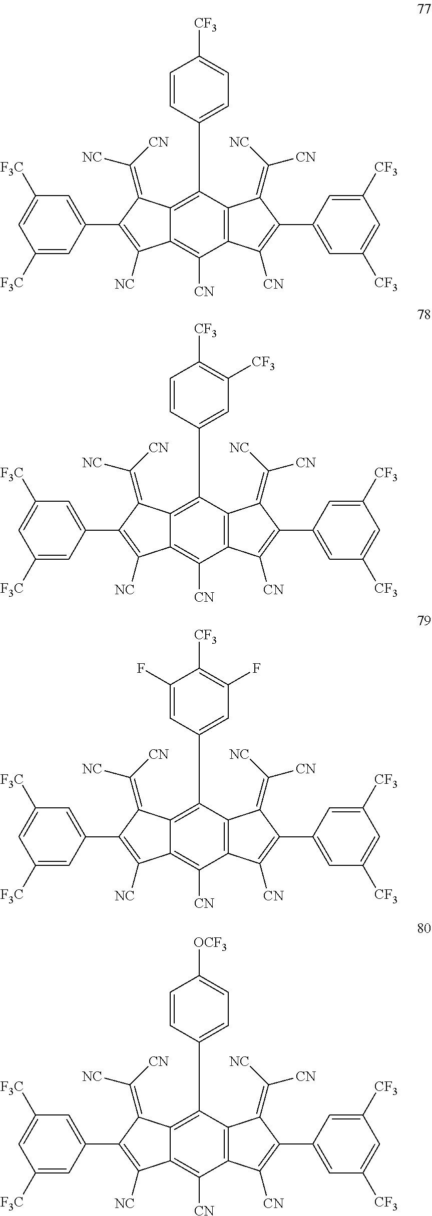

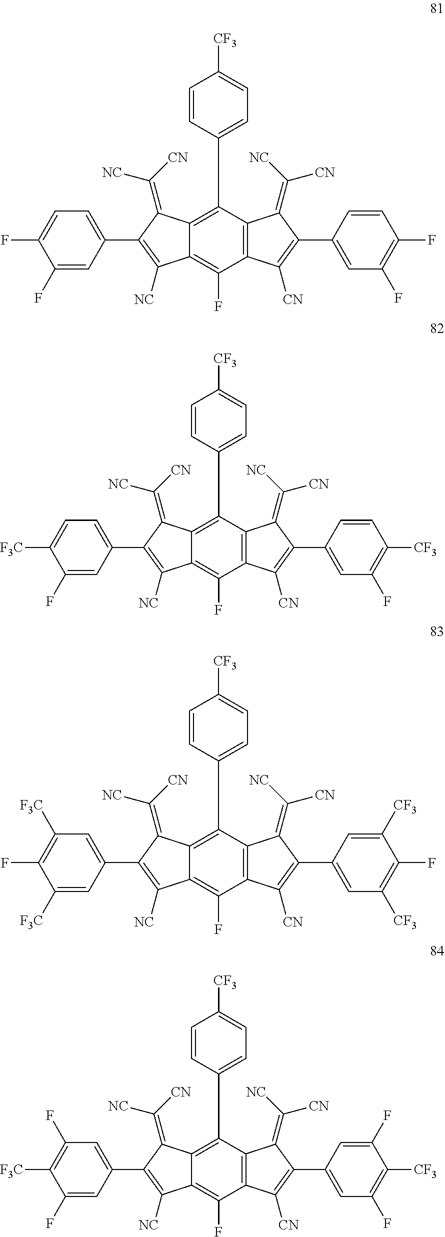

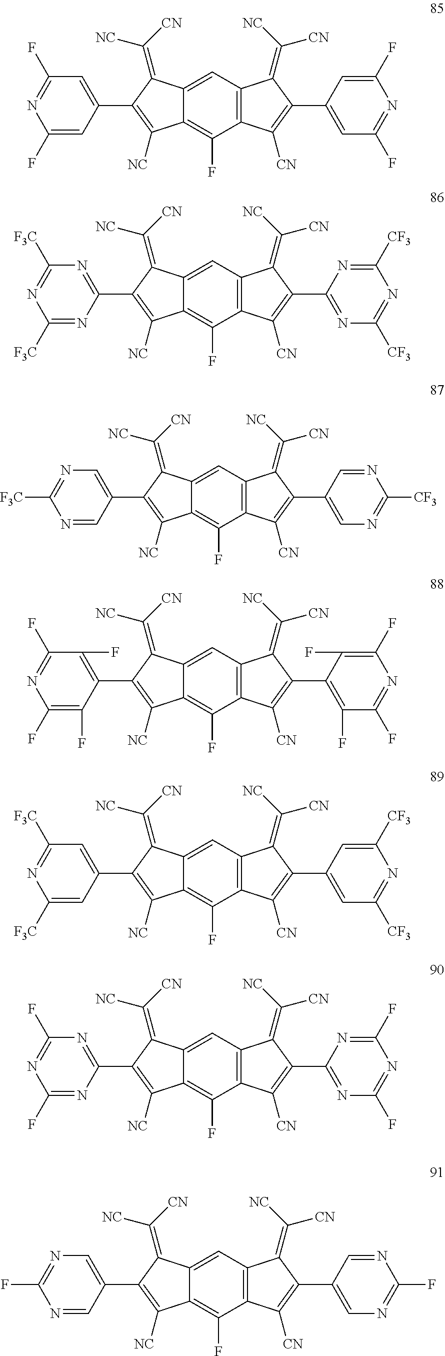

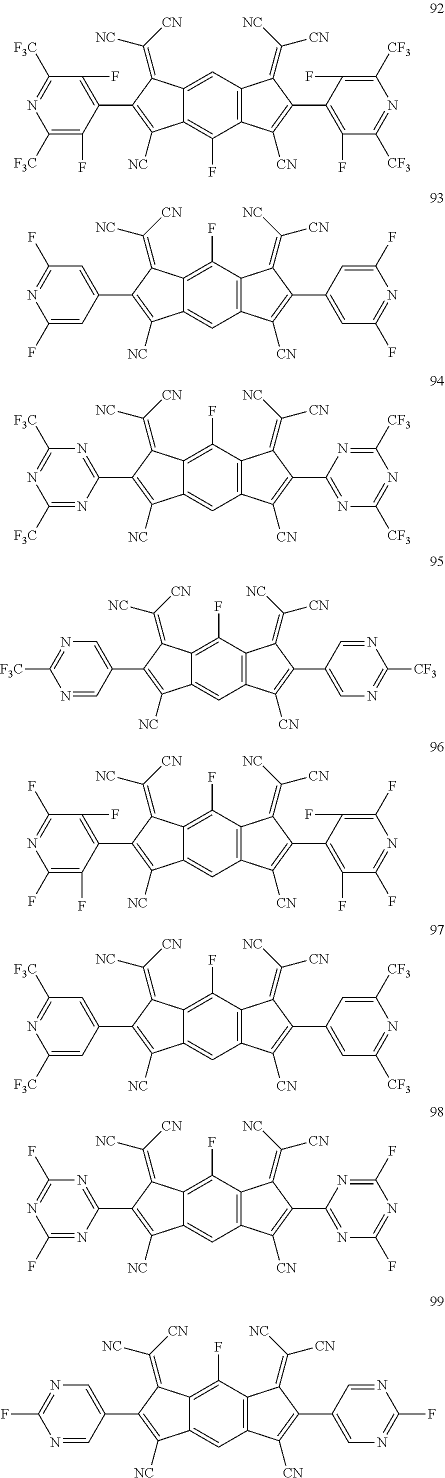

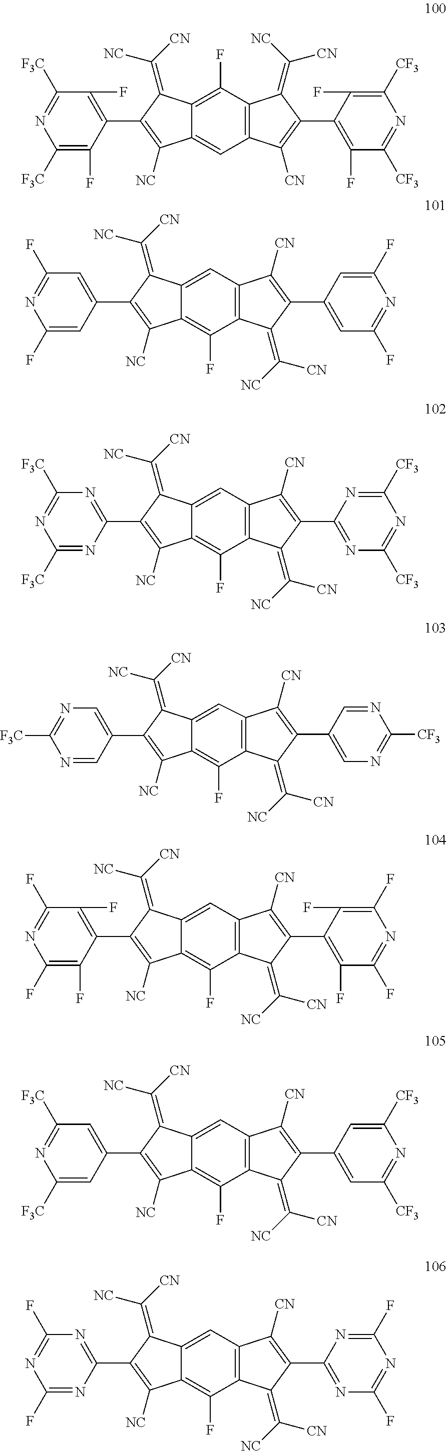

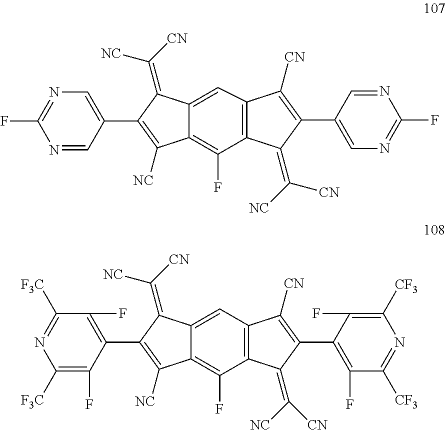 [SMILES: N#CC(C#N)=C1C(c2cnc(F)nc2)=C(C#N)c2c1cc1c(c2F)C(=C(C#N)C#N)C(c2cnc(F)nc2)=C1C#N ; N#CC(C#N)=C1C(c2c(F)c(C(F)(F)F)nc(C(F)(F)F)c2F)=C(C#N)c2c1cc1c(c2F)C(=C(C#N)C#N)C(c2c(F)c(C(F)(F)F)nc(C(F)(F)F)c2F)=C1C#N]

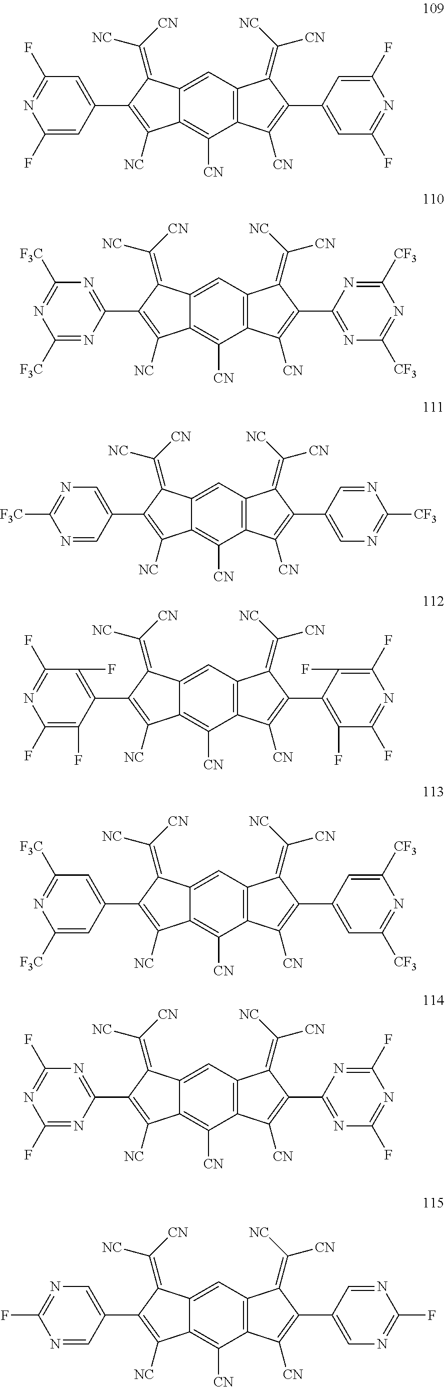

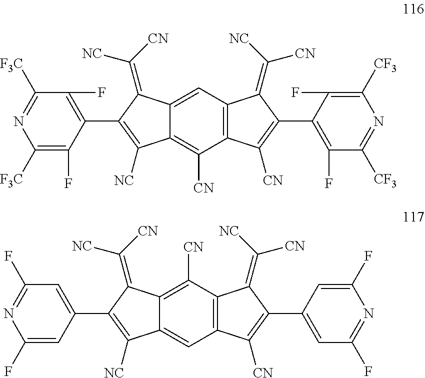 [SMILES: N#CC(C#N)=C1C(c2c(F)c(C(F)(F)F)nc(C(F)(F)F)c2F)=C(C#N)c2c1cc1c(c2C#N)C(C#N)=C(c2c(F)c(C(F)(F)F)nc(C(F)(F)F)c2F)C1=C(C#N)C#N ; N#CC(C#N)=C1C(c2cc(F)nc(F)c2)=C(C#N)c2cc3c(c(C#N)c21)C(=C(C#N)C#N)C(c1cc(F)nc(F)c1)=C3C#N]

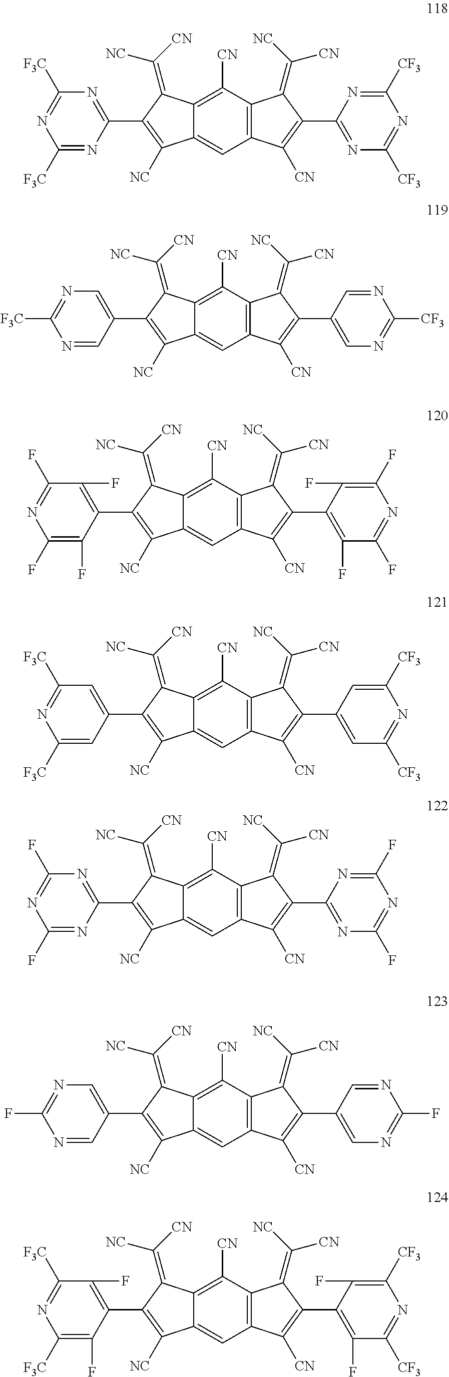

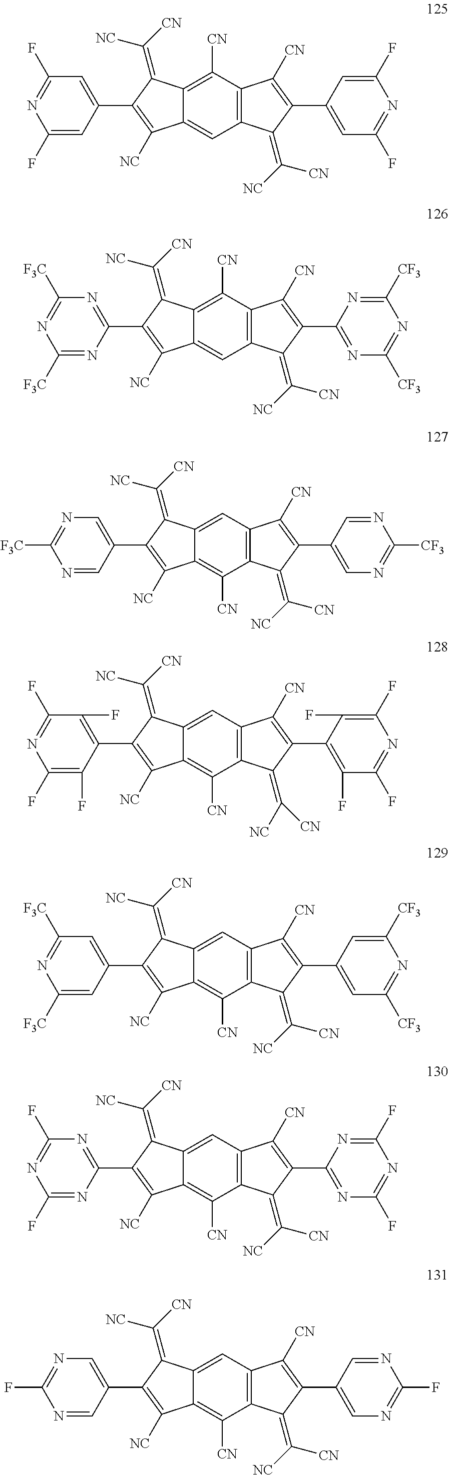

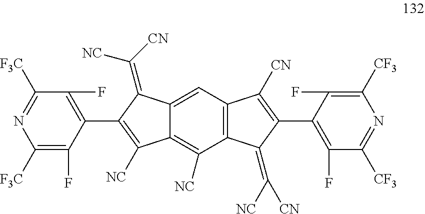 [SMILES: N#CC(C#N)=C1C(c2c(F)c(C(F)(F)F)nc(C(F)(F)F)c2F)=C(C#N)c2c1cc1c(c2C#N)C(=C(C#N)C#N)C(c2c(F)c(C(F)(F)F)nc(C(F)(F)F)c2F)=C1C#N]

Other embodiments of the disclosure may provide a display device. The display device may include the organic light emitting 5 elements 200, 300, 400, 500, 600, 700, 800, and 900 described above with reference to FIGS. 1 to 6.

Embodiments of the disclosure described above are briefly described below.

An organic light emitting element 200, 300, 400, 500, 600, 700, 800, or 900 according to embodiments of the disclosure may comprise a first electrode 210, 310, 410, 510, 610, 710, 810, or 910, a second electrode 220, 320, 420, 520, 620, 720, 820, or 920, and an organic material layer 230, 330, 430, 530, 630, 730, 830, or 930. The organic material layer 230, 330, 430, 530, 630, 730, 830, or 930 may include a compound 232a, 332a, 432a, 532a, 632a, 732a, 832a, or 932a represented by chemical formula 1 described above.

The organic material layer 330, 430, 530, 630, 730, 830, and 930 may include a first light emitting layer 331, 431, 531, 631, 731, 831, or 931 and a first layer 332, 432, 532, 632, 732, 832, or 932. The first layer 332, 432, 532, 632, 732, 832, or 932 may include the above-described compound 332a, 432a, 532a, 632a, 732a, 832a, or 932a.

The first electrode 310, 410, 510, 610, 710, 810, or 910 may be an anode electrode, the second electrode 320, 420, 520, 620, 720, 820, or 920 may be a cathode electrode, and the first layer 332, 432, 532, 632, 732, 832, or 932 may be positioned between the first electrode 310, 410, 510, 610, 710, 810, or 910 and the first light emitting layer 331, 431, 531, 631, 731, 831, or 931. The first layer 332, 432, 532, 632, 732, 832, or 932 may be a hole injection layer, a hole transport layer, or a charge generation layer.

The organic material layer 430, 530, 630, 730, 830, or 930 may include a second light emitting layer 433, 533, 633, 733, 833, or 933, and the first layer 432, 632, 732, 832, or 932 may be positioned between the first light emitting layer 431, 531, 631, 731, 831, or 931 and the second light emitting layer 433, 533, 633, 733, 833, or 933.

The first layer 432, 732, 832, or 932 may be a charge generation layer. In this example, the first layer 432, 732, 832, or 932 may be a p-type charge generation layer.

The compound may be a p dopant of the first layer 432, 532, 732, 832, or 932.

The organic material layer 830 or 930 may further include a third light emitting layer 834 or 934 and a charge generation layer 8351 and 8352 or 9351 and 932. The charge generation layer 8351 and 8352 or 9351 and 932 may be positioned between the second light emitting layer 833 or 933 and the third light emitting layer 834 or 934.

The charge generation layer 8351 and 8352 or 9351 and 932 positioned between the second light emitting layer 833 or 933 and the third light emitting layer 834 or 934 may include the compound 832b or 932b. The compound 832b or 932b may be represented by chemical formula 1.

The display device 100 according to embodiments includes an organic light emitting element 200, 300, 400, 500, 600, 700, 800, or 900.

An example of manufacturing an organic light emitting element according to embodiments of the disclosure are described below in detail with reference to embodiments thereof, but embodiments of the disclosure are not limited to the following embodiments.

### Compound Synthesis Examples

### Preparation Example 1-1. Synthesis of Compound 6-A

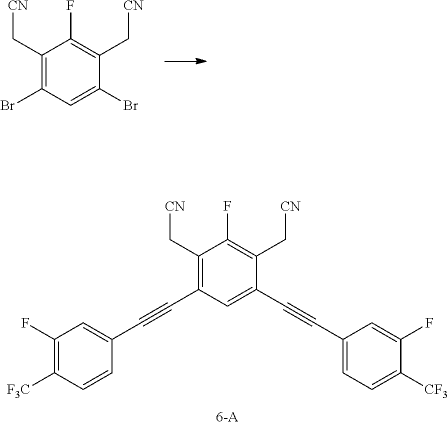 [SMILES: N#CCc1c(Br)cc(Br)c(CC#N)c1F ; N#CCc1c(C#Cc2ccc(C(F)(F)F)c(F)c2)cc(C#Cc2ccc(C(F)(F)F)c(F)c2)c(CC#N)c1F]

20.9 g (63.1 mmol) of 2,2′-(1,3-dibromo-5-fluoro-4,6-phenylene) diacetonitrile, 250 mL of toluene, 12.6 mmol of copper iodide, 12.6 mmol of tetrakistriphenylphosphine palladium, 315.5 mmol of diisopropylamine, and 62.7 mmol of 4-ethynyl-2-fluoro-1-(trifluoromethyl) benzene were mixed, heated to 100° C., and stirred for 2 hours. After the reaction was complete, 150 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 15.5 g of compound 6-A (yield 45%, MS [M+H]=547).

### Preparation Example 1-2. Synthesis of Compound 6-B

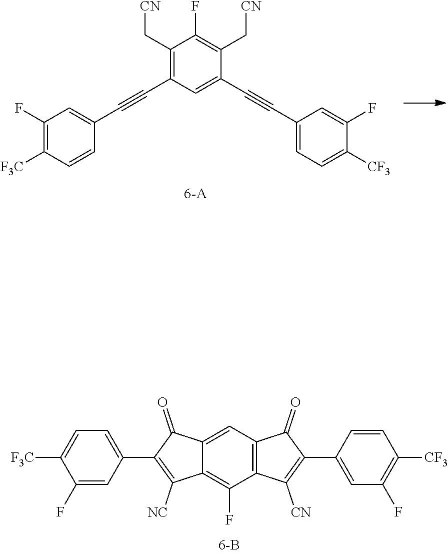 [SMILES: N#CCc1c(C#Cc2ccc(C(F)(F)F)c(F)c2)cc(C#Cc2ccc(C(F)(F)F)c(F)c2)c(CC#N)c1F ; N#Cc1c(-c2ccc(C(F)(F)F)c(F)c2)c(=O)c2cc3c(=O)c(-c4ccc(C(F)(F)F)c(F)c4)c(C#N)c3c(F)c12]

13.9 g (25.4 mmol) of 6-A, 180 mL of 1,4-dioxane, 152.4 mmol of diphenyl sulfoxide, 5.1 mmol of copper bromide (II), and 5.1 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 5.1 g of compound 6-B (yield 35%, MS [M+H]=575).

### Preparation Example 1-3. Synthesis of Compound 6

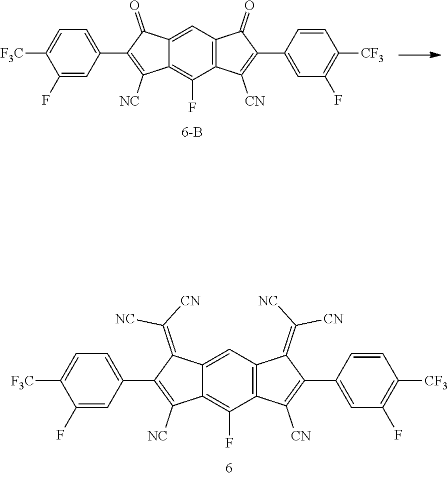 [SMILES: N#Cc1c(-c2ccc(C(F)(F)F)c(F)c2)c(=O)c2cc3c(=O)c(-c4ccc(C(F)(F)F)c(F)c4)c(C#N)c3c(F)c12 ; N#CC(C#N)=C1C(c2ccc(C(F)(F)F)c(F)c2)=C(C#N)c2c1cc1c(c2F)C(C#N)=C(c2ccc(C(F)(F)F)c(F)c2)C1=C(C#N)C#N]

4.3 g (7.5 mmol) of 6-B, 140 mL of dichloromethane, and 52.5 mmol of malononitrile were added and cooled to 0° C. After slowly adding 37.5 mmol of titanium chloride (IV) at 0° C., the mixture was stirred for 1 hour while maintaining the temperature at 0° C. 52.5 mmol of pyridine was dissolved in 40 mL dichloromethane, and then added slowly to the mixture at 0° C., and then the mixture was stirred for one hour while maintaining the temperature. After the reaction was complete, 52.5 mmol of acetic acid was added and the solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 2.0 g of compound 6 (yield 40%, MS [M+H]=671).

### Preparation Example 2-1. Synthesis of Compound 10-A

37.8 g (113.8 mmol) of 2,2′-(1,3-dibromo-5-fluoro-4,6-phenylene) diacetonitrile, 350 mL of toluene, 22.8 mmol of copper iodide, 22.8 mmol of tetrakistriphenylphosphine palladium, 569 mmol of diisopropylamine, and 341.4 mmol of 4-ethynyl-2-(trifluoromethyl) benzonitrile were mixed, heated to 100° C., and stirred for 2 hours. After the reaction was complete, 250 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was off distilled again, and recrystallization was performed twice with tetrahydrofuran/ethanol to obtain 25.5 g of compound 10-A (yield 40%, MS [M+H]=561).

### Preparation Example 2-2. Synthesis of Compound 10-B

14.6 g (26.0 mmol) of 10-A, 200 mL of 1,4-dioxane, 156.0 mmol of diphenyl sulfoxide, 5.2 mmol of copper bromide (II), and 5.2 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 5.2 g of compound 10-B (yield 34%, MS [M+H]=589).

### Preparation Example 2-3. Synthesis of Compound 10

4.0 g (6.8 mmol) of 10-B, 130 mL of dichloromethane, and 47.6 mmol of malononitrile were added and cooled to 0° C. After slowly adding 34 mmol of titanium chloride (IV) at 0° C., the mixture was stirred for 1 hour while maintaining the temperature at 0° C. 47.6 mmol of pyridine was dissolved in 40 mL dichloromethane, and then added slowly to the mixture at 0° C., and then the mixture was stirred for one hour while maintaining the temperature. After the reaction was complete, 47.6 mmol of acetic acid was added and the solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.4 g of compound 10 (yield 30%, MS [M+H]=685).

### Preparation Example 3-1. Synthesis of Compound 22-A

27.9 g (83.9 mmol) of 2,2′-(1,4-dibromo-2-fluoro-3,6-phenylene) diacetonitrile, 300 mL of toluene, 16.8 mmol of copper iodide, 16.8 mmol of tetrakistriphenylphosphine palladium, 419.5 mmol of diisopropylamine, and 251.7 mmol of 4-ethynyl-2-fluoro-1-(trifluoromethyl) benzene were mixed, heated to 100° C., and stirred for 2 hours. After the reaction was complete, 200 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 16.5 g of compound 22-A (yield 36%, MS [M+H]=547).

### Preparation Example 3-2. Synthesis of Compound 22-B

12.7 g (23.2 mmol) of 22-A, 160 mL of 1,4-dioxane, 139.2 mmol of diphenyl sulfoxide, 4.6 mmol of copper bromide (II), and 4.6 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.0 g of compound 22-B (yield 30%, MS [M+H]=575).

### Preparation Example 3-3. Synthesis of Compound 22

3.2 g (5.5 mmol) of 22-B, 100 mL of dichloromethane, and 38.5 mmol of malononitrile were added and cooled to 0° C. After slowly adding 27.5 mmol of titanium chloride (IV) at 0° C., the mixture was stirred for 1 hour while maintaining the temperature at 0° C. 38.5 mmol of pyridine was dissolved in 30 mL dichloromethane, and then added slowly to the mixture at 0° C., and then the mixture was stirred for one hour while maintaining the temperature. After the reaction was complete, 38.5 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.3 g of compound 22 (yield 35%, MS [M+H]=671).

### Preparation Example 4-1. Synthesis of Compound 26-A

28.6 g (86.1 mmol) of 2,2′-(1,4-dibromo-2-fluoro-3,6-phenylene) diacetonitrile, 300 mL of toluene, 17.2 mmol of copper iodide, 17.2 mmol of tetrakistriphenylphosphine palladium, 430.5 mmol of diisopropylamine, and 258.3 mmol of 4-ethynyl-2-(trifluoromethyl) benzonitrile were mixed, heated to 100° C., and stirred for 2 hours. After the reaction was complete, 200 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 18.0 g of compound 26-A (yield 38%, MS [M+H]=551).

### Preparation Example 4-2. Synthesis of Compound 26-B

16.8 g (30.6 mmol) of 26-A, 220 mL of 1,4-dioxane, 183.6 mmol of diphenyl sulfoxide, 6.1 mmol of copper bromide (II), and 6.1 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.5 g of compound 26-B (yield 25%, MS [M+H]=589).

### Preparation Example 4-3. Synthesis of Compound 26

3.9 g (6.6 mmol) of 26-B, 120 mL of dichloromethane, and 46.2 mmol of malononitrile were added and cooled to 0° C. After slowly adding 33 mmol of titanium chloride (IV) at 0° C., the mixture was stirred for 1 hour while maintaining the temperature at 0° C. 46.2 mmol of pyridine was dissolved in 40 mL dichloromethane, and then added slowly to the mixture at 0° C., and then the mixture was stirred for one hour while maintaining the temperature. After the reaction was complete, 46.2 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.4 g of compound 26 (yield 31%, MS [M+H]=685).

### Preparation Example 5-1. Synthesis of Compound 34-A

37.9 g (111.8 mmol) of 2,2′-(1,3-dibromo-5-cyano-4,6-phenylene) diacetonitrile, 400 mL of toluene, 22.4 mmol of copper iodide, 22.4 mmol of tetrakistriphenylphosphine palladium, 559 mmol of diisopropylamine, and 335.4 mmol of 4-ethynyl-2-fluoro-1-(trifluoromethyl) benzene were mixed, heated to 100° C., and stirred for 2 hours. After the reaction was complete, 250 mL of the solvent was distilled, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 20.4 g of compound 34-A (yield 33%, MS [M+H]=554).

### Preparation Example 5-2. Synthesis of Compound 34-B

18.2 g (33.0 mmol) of 34-A, 250 mL of 1,4-dioxane, 198.0 mmol of diphenyl sulfoxide, 6.6 mmol of copper bromide (II), and 6.6 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.6 g of compound 34-B (yield 24%, MS [M+H]=582).

### Preparation Example 5-3. Synthesis of Compound 34

3.4 g (5.9 mmol) of 34-B, 110 mL of dichloromethane, and 41.3 mmol of malononitrile were added and cooled to 0° C. After slowly adding 29.5 mmol of titanium chloride (IV) at 0° C., the mixture was stirred for 1 hour while maintaining the temperature at 0° C. 41.3 mmol of pyridine was dissolved in 35 mL dichloromethane, and then added slowly to the mixture at 0° C., and then the mixture was stirred for one hour while maintaining the temperature. After the reaction was complete, 41.3 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.0 g of compound 34 (yield 25%, MS [M+H]=678).

### Preparation Example 6-1. Synthesis of Compound 38-A

60.8 g (179.3 mmol) of 2,2′-(1,3-dibromo-5-cyano-4,6-phenylene) diacetonitrile, 600 mL of toluene, 35.9 mmol of copper iodide, 35.9 mmol of tetrakistriphenylphosphine palladium, 896.5 of diisopropylamine, and 537.9 mmol of 4-ethynyl-2-mmol (trifluoromethyl) benzonitrile were mixed, heated to 100° C., and stirred for 2 hours. After the reaction was complete, 450 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 30.5 g of compound 38-A (yield 30%, MS [M+H]=568).

### Preparation Example 6-2. Synthesis of Compound 38-B

25.4 g (44.8 mmol) of 38-A, 350 mL of 1,4-dioxane, 268.8 mmol of diphenyl sulfoxide, 9.0 mmol of copper bromide (II), and 9.0 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 6.4 g of compound 38-B (yield 24%, MS [M+H]=596).

### Preparation Example 6-3. Synthesis of Compound 38

4.9 g (8.2 mmol) of 38-B, 150 mL of dichloromethane, and 57.4 mmol of malononitrile were added and cooled to 0° C. After slowly adding 41 mmol of titanium chloride (IV) at 0° C., the mixture was stirred for 1 hour while maintaining the temperature at 0° C. 57.4 mmol of pyridine was dissolved in 50 mL dichloromethane, and then added slowly to the mixture at 0° C., and then the mixture was stirred for one hour while maintaining the temperature. After the reaction was complete, 57.4 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.7 g of compound 38 (yield 30%, MS [M+H]=692).

### Preparation Example 7-1. Synthesis of Compound 54-A

72.3 g (213.4 mmol) of 2,2′-(1,4-dibromo-2-cyano-3,6-phenylene) diacetonitrile, 750 mL of toluene, 42.7 mmol of copper iodide, 42.7 mmol of tetrakistriphenylphosphine palladium, 1067.0 mmol of diisopropylamine, and 640.2 mmol of 4-ethynyl-2-fluoro-1-(trifluoromethyl) benzene were mixed, heated to 100° C., and stirred for 2 hours. After the reaction was complete, 600 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 35.4 g of compound 54-A (yield 30%, MS [M+H]=554).

Preparation example 7-2. Synthesis of compound 54-B 28.6 g (51.6 mmol) of 54-A, 400 mL of 1,4-dioxane, 309.6 mmol of diphenyl sulfoxide, 10.3 mmol of copper bromide (II), and 10.3 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 6.0 g of compound 54-B (yield 20%, MS [M+H]=582).

### Preparation Example 7-3. Synthesis of Compound 54

4.3 g (7.4 mmol) of 54-B, 150 mL of dichloromethane, and 51.8 mmol of malononitrile were added and cooled to 0° C. After slowly adding 37.0 mmol of titanium chloride (IV) at 0° C., the mixture was stirred for 1 hour while maintaining the temperature at 0° C. 51.8 mmol of pyridine was dissolved in 40 mL dichloromethane, and then added slowly to the mixture at 0° C., and then the mixture was stirred for one hour while maintaining the temperature. After the reaction was complete, 51.8 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.2 g of compound 54 (yield 24%, MS [M+H]=678).

### Preparation Example 8-1. Synthesis of Compound 61-A

133.7 g (334.4 mmol) of 2,2′-(1,3-dibromo-2-trifluoromethyl-5-fluoro-4,6-phenylene) diacetonitrile, 1.5 L of toluene, 66.9 mmol of copper iodide, 66.9 mmol of tetrakistriphenylphosphine palladium, 1672 mmol of diisopropylamine, and 1003.2 mmol of 4-ethynyl-2-(trifluoromethyl) benzonitrile were mixed, heated to 100° C., and stirred for 2 hours. After the reaction was complete, 1.3 L of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 42 g of compound 61-A (yield 20%, MS [M+H]=629).

### Preparation Example 8-2. Synthesis of Compound 61-B

35.6 g (56.6 mmol) of 61-A, 500 mL of 1,4-dioxane, 339.6 mmol of diphenyl sulfoxide, 11.3 mmol of copper bromide (II), and 11.3 mmol of palladium acetate were mixed, heated to 100° C., and stirred for 5 hours. After the reaction, the solvent was distilled off, dissolved in chloroform, acid clay was added, and stirred for one hour. After filtering the stirred solution, the solvent was distilled off again and reverse-precipitated was performed using hexane to obtain a solid. The obtained solid was recrystallized with tetrahydrofuran/hexane and filtered to obtain 5.2 g of compound 61-B (yield 14%, MS [M+H]=657).

### Preparation Example 8-3. Synthesis of Compound 61

4.4 g (6.6 mmol) of 61-B, 150 mL of dichloromethane, and 46.2 mmol of malononitrile were added and cooled to 0° C. After slowly adding 33 mmol of titanium chloride (IV) at 0° C., the mixture was stirred for 1 hour while maintaining the temperature at 0° C. 46.2 mmol of pyridine was dissolved in 40 mL dichloromethane, and then added slowly to the mixture at 0° C., and then the mixture was stirred for one hour while maintaining the temperature. After the reaction was complete, 46.2 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.0 g of compound 61 (yield 20%, MS [M+H]=753).

### Preparation Example 9-1. Synthesis of Compound 66-A

61.1 g (152.7 mmol) of 2,2′-(1,3-dibromo-2-trifluoromethyl-5-fluoro-4,6-phenylene) diacetonitrile, 600 mL of toluene, 30.5 mmol of copper iodide, 30.5 mmol of tetrakistriphenylphosphine palladium, 763.5 mmol of diisopropylamine, and 458.1 mmol of 4-ethynyl-2-fluoro-1-(trifluoromethyl) benzene were mixed, heated to 100° C., and stirred for 2 hours. After the reaction was complete, 500 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 30.0 g of compound 66-A (yield 32%, MS [M+H]=615).

### Preparation Example 9-2. Synthesis of Compound 66-B

22.2 g (63.1 mmol) of 66-A, 300 ml of 1,4-dioxane, 378.6 mmol of diphenyl sulfoxide, 12.6 mmol of copper bromide (II), and 12.6 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 5.8 g of compound 66-B (yield 25%, MS [M+H]=643).

### Preparation Example 9-3. Synthesis of Compound 66

4.7 g (7.3 mmol) of 66-B, 160 mL of dichloromethane, and 51.1 mmol of malononitrile were added and cooled to 0° C. After slowly adding 36.5 mmol of titanium chloride (IV) at 0° C., the mixture was stirred for 1 hour while maintaining the temperature at 0° C. 51.1 mmol of pyridine was dissolved in 50 mL dichloromethane, and then added slowly to the mixture at 0° C., and then the mixture was stirred for one hour while maintaining the temperature. After the reaction was complete, 51.1 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.5 g of compound 66 (yield 28%, MS [M+H]=739).

### Preparation Example 10-1. Synthesis of Compound 69-A

40.4 g (101.0 mmol) of 2,2′-(1,3-dibromo-2-trifluoromethyl-5-fluoro-4,6-phenylene) diacetonitrile, 400 mL of toluene, 20.2 mmol of copper iodide, 20.2 mmol of tetrakistriphenylphosphine palladium, 505.0 mmol of diisopropylamine, and 303.0 mmol of 4-ethynyl-2-(trifluoromethoxy) benzonitrile were mixed, heated to 100° C., and stirred for 2 hours. After the reaction was complete, 300 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 22.0 g of compound 69-A (yield 33%, MS [M+H]=661).

### Preparation Example 10-2. Synthesis of Compound 69-B

16.1 g (24.4 mmol) of 69-A, 200 mL of 1,4-dioxane, 146.4 mmol of diphenyl sulfoxide, 4.9 mmol of copper bromide (II), and 4.9 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.2 g of compound 69-B (yield 25%, MS [M+H]=689).

### Preparation Example 10-3. Synthesis of Compound 69

3.8 g (5.5 mmol) of 69-B, 120 mL of dichloromethane, and 38.5 mmol of malononitrile were added and cooled to 0° C. After slowly adding 27.5 mmol of titanium chloride (IV) at 0° C., the mixture was stirred for 1 hour while maintaining the temperature at 0° C. 38.5 mmol of pyridine was dissolved in 40 mL dichloromethane, and then added slowly to the mixture at 0° C., and then the mixture was stirred for one hour while maintaining the temperature. After the reaction was complete, 38.5 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.0 g of compound 69 (yield 23%, MS [M+H]=785).

### Preparation Example 11-1. Synthesis of Compound 82-A

52.3 g (109.9 mmol) of 2,2′-(1,3-dibromo-2-(4-(trifluoromethyl) phenyl)-5-fluoro-4,6-phenylene) diacetonitrile, 600 mL of toluene, 22 mmol of copper iodide, 22 mmol of tetrakistriphenylphosphine palladium, 550 mmol of diisopropylamine, and 329.7 mmol of 4-ethynyl-2-fluoro-1-(trifluoromethyl) benzene were mixed, heated to 100° C., and stirred for 2 hours. After the reaction was complete, 500 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 23.5 g of compound 82-A (yield 31%, MS [M+H]=691).

### Preparation Example 11-2. Synthesis of Compound 82-B

20.3 g (29.4 mmol) of 82-A, 300 mL of 1,4-dioxane, 176.4 mmol of diphenyl sulfoxide, 5.9 mmol of copper bromide (II), and 5.9 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 3.8 g of compound 82-B (yield 18%, MS [M+H]=719).

### Preparation Example 11-3. Synthesis of Compound 82

3.4 g (4.7 mmol) of 82-B, 120 mL of dichloromethane, and 32.9 mmol of malononitrile were added and cooled to 0° C. After slowly adding 23.5 mmol of titanium chloride (IV) at 0° C., the mixture was stirred for 1 hour while maintaining the temperature at 0° C. 32.9 mmol of pyridine was dissolved in 35 mL dichloromethane, and then added slowly to the mixture at 0° C., and then the mixture was stirred for one hour while maintaining the temperature. After the reaction was complete, 32.9 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 0.9 g of compound 82 (yield 23%, MS [M+H]=831).

### Preparation Example 12-1. Synthesis of Compound 85-A

35.6 g (107.3 mmol) of 2,2′-(1,3-dibromo-5-fluoro-4,6-phenylene) diacetonitrile, 400 mL of toluene, 21.5 mmol of copper iodide, 21.5 mmol of tetrakistriphenylphosphine palladium, 536.5 mmol of diisopropylamine, and 321.9 mmol 4-ethynyl-2,6-difluoropyridine were mixed, heated to 100° C., and stirred for 2 hours. After the reaction was complete, 250 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 20.0 g of compound 85-A (yield 42%, MS [M+H]=445).

### Preparation Example 12-2. Synthesis of Compound 85-B

18.7 g (42.0 mmol) of 85-A, 250 mL of 1,4-dioxane, 252.0 mmol of diphenyl sulfoxide, 8.4 mmol of copper bromide (II), and 8.4 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 7.0 g of compound 85-B (yield 35%, MS [M+H]=477).

### Preparation Example 12-3. Synthesis of Compound 85

5.5 g (11.4 mmol) of 85-B, 160 mL of dichloromethane, and 79.8 mmol of malononitrile were added and cooled to 0° C. After slowly adding 57.0 mmol of titanium chloride (IV) at 0° C., the mixture was stirred for 1 hour while maintaining the temperature at 0° C. 79.8 mmol of pyridine was dissolved in 50 mL dichloromethane, and then added slowly to the mixture at 0° C., and then the mixture was stirred for one hour while maintaining the temperature. After the reaction was complete, 79.8 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 2.1 g of compound 85 (yield 32%, MS [M+H]=573).

### Preparation Example 13-1. Synthesis of Compound 101-A

46.7 g (140.8 mmol) of 2,2′-(1,4-dibromo-2-fluoro-3,6-phenylene) diacetonitrile, 500 mL of toluene, 28.2 mmol of copper iodide, 28.2 mmol of tetrakistriphenylphosphine palladium, 704.0 mmol of diisopropylamine, and 422.4 mmol of 4-ethynyl-2,6-difluoropyridine were mixed, heated to 100° C., and stirred for 2 hours. After the reaction was complete, 400 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 25.0 g of compound 101-A (yield 40%, MS [M+H]=445).

### Preparation Example 13-2. Synthesis of Compound 101-B

19.2 g (43.3 mmol) of 101-A, 250 mL of 1,4-dioxane, 259.8 mmol of diphenyl sulfoxide, 8.7 mmol of copper bromide (II), and 8.7 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 6.6 g of compound 101-B (yield 32%, MS [M+H]=477).

### Preparation Example 13-3. Synthesis of Compound 101

5.3 g (11.2 mmol) of 101-B, 180 mL of dichloromethane, and 78.4 mmol of malononitrile were added and cooled to 0° C. After slowly adding 56.0 mmol of titanium chloride (IV) at 0° C., the mixture was stirred for 1 hour while maintaining the temperature at 0° C. 78.4 mmol of pyridine was dissolved in 50 mL dichloromethane, and then added slowly to the mixture at 0° C., and then the mixture was stirred for one hour while maintaining the temperature. After the reaction was complete, 78.4 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was again using acetonitrile/tert-recrystallized butylmethylether and purified by sublimation, obtaining 1.8 g of compound 101 (yield 28%, MS [M+H]=573).

### Preparation Example 14-1. Synthesis of Compound 109-A

91.5 g (270.0 mmol) of 2,2′-(1,3-dibromo-5-cyano-4,6-phenylene) diacetonitrile, 1 L of toluene, 54.0 mmol of copper iodide, 54.0 mmol of tetrakistriphenylphosphine palladium, 1350.0 mmol of diisopropylamine, and 810.0 of 4-ethynyl-2,6-difluoropyridine were mixed, heated to 100° C., and stirred for 2 hours. After the reaction was complete, 800 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 43.0 g of compound 109-A (yield 35%, MS [M+H]=456).

### Preparation Example 14-2. Synthesis of Compound 109-B

34.4 g (75.6 mmol) of 109-A, 500 mL of 1,4-dioxane, 453.6 mmol of diphenyl sulfoxide, 15.1 mmol of copper bromide (II), and 15.1 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 7.3 g of compound 109-B (yield 20%, MS [M+H]=484).

### Preparation Example 14-3. Synthesis of Compound 109

5.0 g (10.4 mmol) of 109-B, 180 mL of dichloromethane, and 72.8 mmol of malononitrile were added and cooled to 0° C. After slowly adding 52.0 mmol of titanium chloride (IV) at 0° C., the mixture was stirred for 1 hour while maintaining the temperature at 0° C. 72.8 mmol of pyridine was dissolved in 50 mL dichloromethane, and then added slowly to the mixture at 0° C., and then the mixture was stirred for one hour while maintaining the temperature. After the reaction was complete, 72.8 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.5 g of compound 109 (yield 25%, MS [M+H]=580).

### Preparation Example 15-1. Synthesis of Compound 125-A

67.3 g (198.5 mmol) of 2,2′-(1,4-dibromo-2-cyano-3,6-phenylene) diacetonitrile, 700 mL of toluene, 39.7 mmol of copper iodide, 39.7 mmol of tetrakistriphenylphosphine palladium, 992.5 mmol of diisopropylamine, and 595.5 mmol of 4-ethynyl-2,6-difluoropyridine were mixed, heated to 100° C., and stirred for 2 hours. After the reaction was complete, 600 mL of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 28.0 g of compound 125-A (yield 31%, MS [M+H]=456).

### Preparation Example 15-2. Synthesis of Compound 125-B

25.1 g (55.2 mmol) of 125-A, 350 mL of 1,4-dioxane, 331.2 mmol of diphenyl sulfoxide, 11.0 mmol of copper bromide (II), and 11.0 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.0 g of compound 125-B (yield 15%, MS [M+H]=484).

### Preparation Example 15-3. Synthesis of Compound 125

3.5 g (7.2 mmol) of 125-B, 120 mL of dichloromethane, and 50.4 mmol of malononitrile were added and cooled to 0° C. After slowly adding 36.0 mmol of titanium chloride (IV) at 0° C., it was stirred for 1 hour while maintaining the temperature at 0° C. 50.4 mmol of pyridine was dissolved in 30 mL dichloromethane, and then added slowly to the mixture at 0° C., and then the mixture was stirred for one hour while maintaining the temperature. After the reaction was complete, 50.4 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. After filtering the solution, it was recrystallized with acetonitrile/toluene and washed with toluene. The obtained solid was recrystallized again using acetonitrile/tert-butylmethylether and purified by sublimation, obtaining 1.0 g of compound 125 (yield 24%, MS [M+H]=580).

### [Manufacturing Evaluation of Organic Light Emitting Element]

A hole injection layer (HIL, 80 Å, NPD doped with 10 wt % of the HIL compound shown in Table 1 below), a hole transport layer (HTL, 950 Å, NPD), the light emitting material layer (EML, 250 Å, host (9,10-di(naphtha-2-yl) anthracene)+dopant (1,6-bis (diphenylamine) pyrene, 3 wt %), an electron transport layer (ETL, 200 Å, TmPyPB), an electron injection layer (EIL, LiF, 10 Å), and a cathode (Al, 2000 Å) were sequentially stacked on the ITO (anode), forming an organic light emitting device.

TABLE 1

| | Organic | | Efficiency | T95 |
|---|---|---|---|---|
| | electron | Voltage | (relative | (relative |
| | acceptor | (V) | value, %) | value, %) |
| comparison example 1 | HATCN | 5.1 | 100 | 100 |
| comparison example 2 | PD1 | 4.0 | 108 | 111 |
| comparison example 3 | PD2 | 4.0 | 109 | 110 |
| comparison example 4 | PD3 | 3.8 | 116 | 106 |
| comparison example 5 | PD4 | 3.8 | 118 | 102 |
| embodiment 1 | 6 | 3.9 | 120 | 125 |
| embodiment 2 | 10 | 3.9 | 119 | 120 |
| embodiment 3 | 22 | 3.8 | 123 | 131 |
| embodiment 4 | 26 | 3.7 | 126 | 142 |
| embodiment 5 | 34 | 3.9 | 121 | 128 |
| embodiment 6 | 38 | 3.9 | 122 | 127 |
| embodiment 7 | 54 | 3.8 | 121 | 130 |
| embodiment 8 | 61 | 3.7 | 128 | 140 |
| embodiment 9 | 66 | 3.7 | 126 | 145 |
| embodiment 10 | 69 | 3.8 | 120 | 136 |
| embodiment 11 | 82 | 3.8 | 123 | 134 |
| embodiment 12 | 85 | 3.8 | 122 | 131 |
| embodiment 13 | 101 | 3.9 | 120 | 128 |
| embodiment 14 | 109 | 3.8 | 124 | 122 |
| embodiment 15 | 125 | 3.9 | 121 | 120 |

HATCN of comparative example 1 of Table 1 is 1,4,5,8,9,11-hexaazatriphenylenehexacarbonitile. The PD1 to PD4 compounds of comparative examples 2 and 3 are as follows.

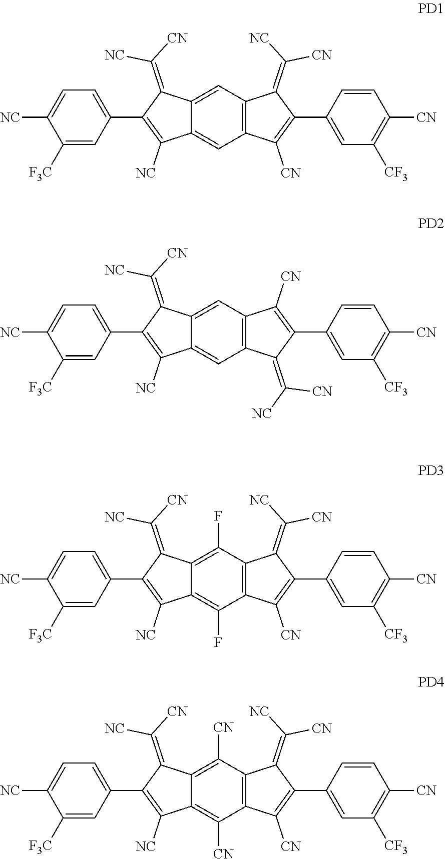 [SMILES: N#CC(C#N)=C1C(c2ccc(C#N)c(C(F)(F)F)c2)=C(C#N)c2cc3c(cc21)C(=C(C#N)C#N)C(c1ccc(C#N)c(C(F)(F)F)c1)=C3C#N ; N#CC(C#N)=C1C(c2ccc(C#N)c(C(F)(F)F)c2)=C(C#N)c2cc3c(cc21)C(C#N)=C(c1ccc(C#N)c(C(F)(F)F)c1)C3=C(C#N)C#N ; N#CC(C#N)=C1C(c2ccc(C#N)c(C(F)(F)F)c2)=C(C#N)c2c(F)c3c(c(F)c21)C(=C(C#N)C#N)C(c1ccc(C#N)c(C(F)(F)F)c1)=C3C#N ; N#CC(C#N)=C1C(c2ccc(C#N)c(C(F)(F)F)c2)=C(C#N)c2c(C#N)c3c(c(C#N)c21)C(=C(C#N)C#N)C(c1ccc(C#N)c(C(F)(F)F)c1)=C3C#N]

Referring to Table 1, the organic light emitting element of the embodiment which uses the compound represented by chemical formula 1 of the disclosure in the hole injection layer has better efficiency, longer lifespan, and lower driving voltage than the organic light emitting element of the comparative example.

In the PD1 and PD2 compounds, benzene bonded to the indacene derivative in the center has a cyano group as a substituent, but the compounds used in the embodiments do not have a cyano group bonded to the corresponding position. Due to this difference, the organic light emitting element according to the embodiments appears to have better efficiency, longer lifespan, and lower driving voltage than the organic light emitting elements of comparative example 2 and comparative example 3.

In the PD3 and PD4 compounds, two electron withdrawing groups are substituted in the benzene portion of the indacene derivative in the center. The organic light emitting element according to embodiments using the compound in which one electron withdrawing group is substituted in the benzene portion of the indacene derivative in the center has better efficiency, longer lifespan or lower driving voltage than organic light emitting elements of comparative example 4 and comparative example 5 using the compound in which two electron withdrawing groups are substituted.

### [Manufacturing Evaluation 2 of Organic Light Emitting Element]

A hole injection layer (HIL, 80 Å, NPD+HATCN (10 wt %)), a first hole transport layer (HTL1, 950 Å, NPD), a first light emitting layer (EML1, 250 Å, host (9,10-di (naphtha-2-yl) anthracene)+dopant (1,6-bis (diphenylamine) pyrene, 3 wt %)), a first electron transport layer (ETL1, 150 Å, 1,3,5-tri(m-pyridin-3-ylphenyl)benzene (TmPyPB)), an n-type charge generation layer (n-CGL, 200 Å, bphen+Li (2 wt %)), a p-type charge generation layer (p-CGL, 150 Å, NPD doped with 20 wt % of p-CGL compound shown in Table 2 below), a second hole transport layer (HTL2, 300 Å, NPD), a second light emitting layer (EML2, 300 Å, host (CBP)+dopant (Ir(ppy)₃, 8 wt %)), a second electron transport layer (ETL2, 200 Å, 2,2′,2″-(1,3,5-benzinetriyl)-tris(1-phenyl-1-H benzimidazole) (TPBi)), an electron injection layer (EIL, LiF, 10 Å) and a cathode (Al, 1500 Å) were sequentially stacked on an ITO (anode), forming an organic light emitting diode.

TABLE 2

| | Organic | | Efficiency | T95 |
|---|---|---|---|---|
| | electron | Voltage | (relative | (relative |
| | acceptor | (V) | value, %) | value, %) |
| comparison example 6 | HATCN | 9.7 | 100 | 100 |
| comparison example 7 | PD1 | 8.1 | 115 | 122 |
| comparison example 8 | PD2 | 8.0 | 117 | 127 |
| comparison example 9 | PD3 | 7.8 | 118 | 115 |
| comparison example 10 | PD4 | 7.8 | 121 | 112 |
| embodiment 16 | 6 | 7.9 | 125 | 136 |
| embodiment 17 | 10 | 7.8 | 126 | 138 |
| embodiment 18 | 22 | 7.8 | 130 | 147 |
| embodiment 19 | 26 | 7.7 | 134 | 156 |
| embodiment 20 | 34 | 7.9 | 124 | 140 |
| embodiment 21 | 38 | 7.9 | 125 | 142 |
| embodiment 22 | 54 | 7.8 | 128 | 146 |
| embodiment 23 | 61 | 7.7 | 137 | 160 |
| embodiment 24 | 66 | 7.6 | 136 | 162 |
| embodiment 25 | 69 | 7.8 | 129 | 150 |
| embodiment 26 | 82 | 7.7 | 132 | 158 |
| embodiment 27 | 85 | 7.8 | 130 | 155 |
| embodiment 28 | 101 | 7.9 | 124 | 149 |
| embodiment 29 | 109 | 7.8 | 131 | 152 |
| embodiment 30 | 125 | 7.9 | 127 | 143 |

HATCN of comparative example 4 of Table 2 is 1,4,5,8,9,11-hexaazatriphenylenehexacarbonitile. The PD1 and PD2 compounds of comparative examples 5 and 6 are the same as the PD1 and PD2 compounds of comparative examples 2 and 3 described above.

Referring to Table 2, the organic light emitting element of the embodiment which uses the compound represented by chemical formula 1 of the disclosure in the p-type charge generation layer has better efficiency, longer lifespan, and lower driving voltage than the organic light emitting element of the comparative example.

In the PD1 and PD2 compounds, benzene bonded to the indacene derivative in the center has a cyano group as a substituent, but the compounds used in the embodiments do not have a cyano group bonded to the corresponding position. Due to this difference, the organic light emitting element according to the embodiments appears to have better efficiency, longer lifespan, and lower driving voltage than the organic light emitting elements of comparative example 7 and comparative example 8.

In the PD3 and PD4 compounds, two electron withdrawing groups are substituted in the benzene portion of the indacene derivative in the center. The organic light emitting element according to embodiments using the compound in which one electron withdrawing group is substituted in the benzene portion of the indacene derivative in the center has better efficiency, longer lifespan or lower driving voltage than organic light emitting elements of comparative example 4 and comparative example 5 using the compound in which two electron withdrawing groups are substituted.

It will be apparent to those skilled in the art that various modifications and variations can be made in the organic light emitting element and the display device of the present disclosure without departing from the technical idea or scope of the disclosure. Thus, it is intended that the present disclosure cover the modifications and variations of this disclosure provided they come within the scope of the appended claims and their equivalents.

What is claimed:

1. An organic light emitting element, comprising:
a first electrode;
a second electrode; and
an organic material layer positioned between the first electrode and the second electrode, wherein the organic material layer includes a compound represented by chemical formula 1 below: 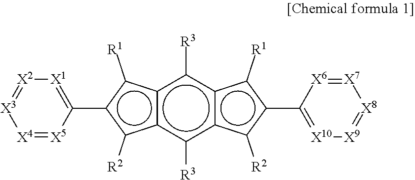
in chemical formula 1,
R¹ and R² are each independently selected from the group consisting of hydrogen; deuterium; tritium; halogen; a cyano group; an alkyl group of C₁-C₅₀; a haloalkyl group of C₁-C₅₀; an alkoxy group of C₁-C₃₀; a haloalkoxy group of C₁-C₃₀; an aryl group of C₆-C₆₀; a haloaryl group of C₆-C₆₀; a heterocyclic group of C₂-C₆₀ including at least one heteroatom among O, N, S, Si and P; a haloheterocyclic group of C₂-C₆₀ including at least one heteroatom among O, N, S, Si and P; and malononitrile,
one R³ is halogen; or a cyano group, and the other R³ is selected from the group consisting of hydrogen; deuterium; tritium; a haloalkyl group of C₁-C₅₀; a haloalkoxy group of C₁-C₃₀; and a haloaryl group of C₆-C₆₀ substituted with one or more of a halogen and a haloalkoxy group of C₁-C₃₀,
X¹ to X⁵ are each independently CR^{a} or N, and at least two of X¹ to X⁵ are CR^{a}, wherein R^{a} is each independently selected from the group consisting of hydrogen; deuterium; tritium; halogen; a cyano group; an alkyl group of C₁-C₅₀; a haloalkyl group of C₁-C₅₀; an alkoxy group of C₁-C₅₀; and a haloalkoxy group of C₁-C₅₀, and at least one of R^{a} is selected from the group consisting of halogen; a cyano group; a haloalkyl group of C₁-C₅₀; and a haloalkoxy group of C₁-C₅₀, and
X⁶ to X¹⁰ are each independently CR^{b} or N, and at least two of X⁶ to X¹⁰ are CR^{b}, wherein R^{b} is each independently selected from the group consisting of hydrogen; deuterium; tritium; halogen; a cyano group; an alkyl group of C₁-C₅₀; a haloalkyl group of C₁-C₅₀; an alkoxy group of C₁-C₅₀; and a haloalkoxy group of C₁-C₅₀, and at least one of R^{b} is selected from the group consisting of halogen; a cyano group; a haloalkyl group of C₁-C₅₀; and a haloalkoxy group of C₁-C₅₀, and
wherein in the alkyl group, the haloalkyl group, the alkoxy group, the haloalkoxy group, the aryl group, the haloaryl group, the heterocyclic group, and the haloheterocyclic group, at least one substituent selected from the group consisting of deuterium; a nitro group; a cyano group; an amino group; an alkoxy group of C₁-C₂₀; a haloalkoxy group of C₁-C₂₀; an alkyl group of C₁-C₂₀; a haloalkyl group of C₁-C₂₀; an alkenyl group of C₂-C₂₀; an alkynyl group of C₂-C₂₀; an aryl group of C₆-C₂₀; an aryl group of C₆-C₂₀ substituted with deuterium; a fluorenyl group; a heterocyclic group of C₂-C₂₀; an alkylsilyl group of C₃-C₆₀; an arylsilyl group of C₁₈-C₆₀; and an alkylarylsilyl group of C₈-C₆₀ may be further substituted.

2. The organic light emitting element of claim 1, wherein the compound is represented by any one of chemical formula 2 and chemical formula 3 below: 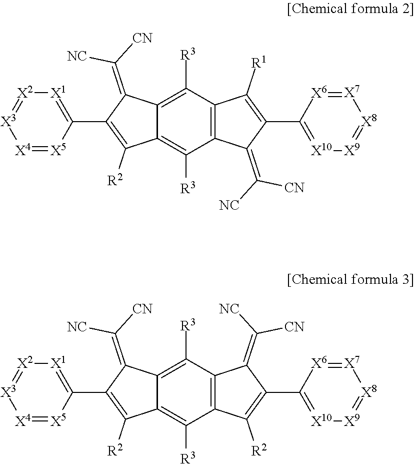
in chemical formula 2 and chemical formula 3, R¹ to R³ and X¹ to X¹⁰ are the same as R¹ to R³ and X¹ to X¹⁰ defined in chemical formula 1.

3. The organic light emitting element of claim 1, wherein the compound is represented by any one of chemical formula 4 and chemical formula 5 below: 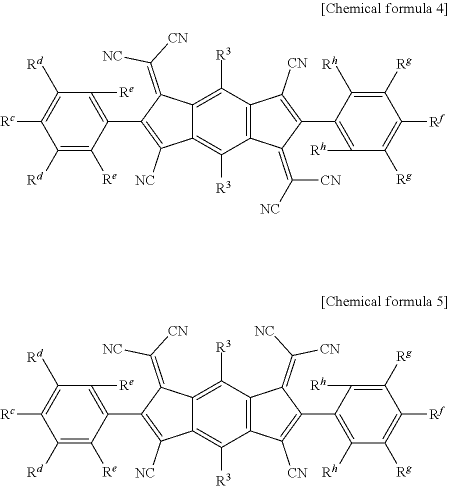
in chemical formula 4 and chemical formula 5, R^{c} to R^{h} are each independently selected from the group consisting of hydrogen; deuterium; tritium; halogen; an alkyl group of C₁-C₅₀; a haloalkyl group of C₁-C₅₀; an alkoxy group of C₁-C₅₀; and a haloalkoxy group of C₁-C₅₀, wherein R³ is the same as R³ defined in chemical formula 1.

4. The organic light emitting element of claim 3, wherein in chemical formula 4,
R^{c} is selected from the group consisting of halogen; a haloalkyl group of C₁-C₅₀; and a haloalkoxy group of C₁-C₅₀,
i) one of two R^{d}s is selected from the group consisting of hydrogen; deuterium; or tritium; and the other R^{d} is selected from the group consisting of halogen; a haloalkyl group of C₁-C₅₀; and a haloalkoxy group of C₁-C₅₀; or
ii) the two R^{d}s are each independently selected from the group consisting of halogen; a haloalkyl group of C₁-C₅₀; and a haloalkoxy group of C₁-C₅₀,
R^{e} is each independently selected from among hydrogen; deuterium; tritium; halogen; a cyano group; a haloalkyl group of C₁-C₅₀; and a haloalkoxy group of C₁-C₃₀,
R^{f} is selected from the group consisting of halogen; a haloalkyl group of C₁-C₅₀; and a haloalkoxy group of C₁-C₅₀,
i) one of two R^{g}s is selected from the group consisting of hydrogen; deuterium; or tritium; and the other R^{g} is selected from the group consisting of halogen; a haloalkyl group of C₁-C₅₀; and a haloalkoxy group of C₁-C₅₀; or
ii) the two R^{g}s are each independently selected from the group consisting of halogen; a haloalkyl group of C₁-C₅₀; and a haloalkoxy group of C₁-C₅₀, and
R^{h} is each independently selected from among hydrogen; deuterium; tritium; halogen; a cyano group; a haloalkyl group of C₁-C₅₀; and a haloalkoxy group of C₁-C₃₀, and
wherein in chemical formula 5,
R^{c} is selected from the group consisting of halogen; a haloalkyl group of C₁-C₅₀; and a haloalkoxy group of C₁-C₅₀,
i) one of two R^{d}s is selected from the group consisting of hydrogen; deuterium; or tritium; and the other R^{d} is selected from the group consisting of halogen; a haloalkyl group of C₁-C₅₀; and a haloalkoxy group of C₁-C₅₀; or
ii) the two R^{d}s are each independently selected from the group consisting of halogen; a haloalkyl group of C₁-C₅₀; and a haloalkoxy group of C₁-C₅₀,
R^{e} is each independently selected from among hydrogen; deuterium; tritium; halogen; a cyano group; a haloalkyl group of C₁-C₅₀; and a haloalkoxy group of C₁-C₃₀,
R^{f} is selected from the group consisting of halogen; a haloalkyl group of C₁-C₅₀; and a haloalkoxy group of C₁-C₅₀, and
i) one of two R^{g}s is selected from the group consisting of hydrogen; deuterium; or tritium; and the other R^{g} is selected from the group consisting of halogen; a haloalkyl group of C₁-C₅₀; and a haloalkoxy group of C₁-C₅₀; or
ii) the two R^{g}s are each independently selected from the group consisting of halogen; a haloalkyl group of C₁-C₅₀; and a haloalkoxy group of C₁-C₅₀, and
R^{h} is each independently selected from among hydrogen; deuterium; tritium; halogen; a cyano group; a haloalkyl group of C₁-C₅₀; and a haloalkoxy group of C₁-C₃₀.

5. The organic light emitting element of claim 1, wherein the compound is represented by any one of chemical formula 6 to chemical formula 15 below: 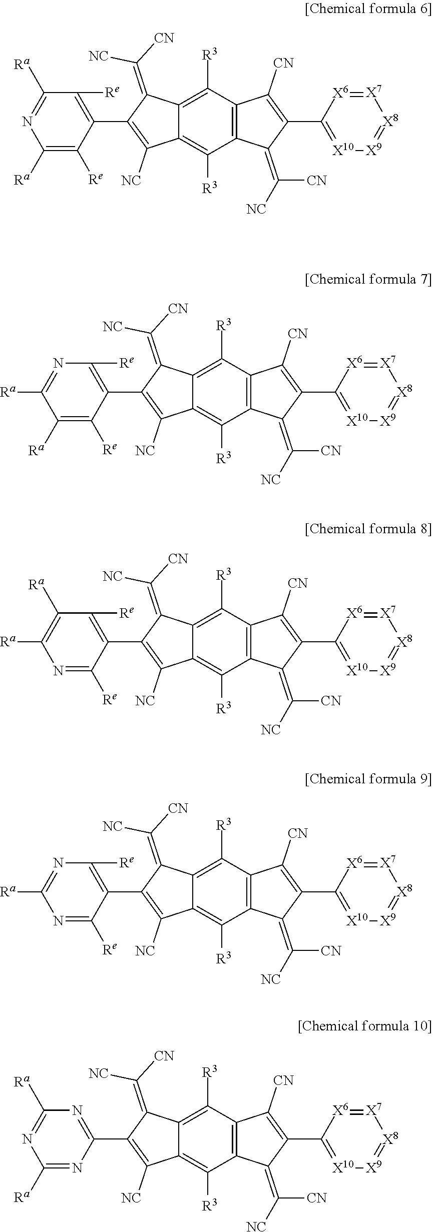 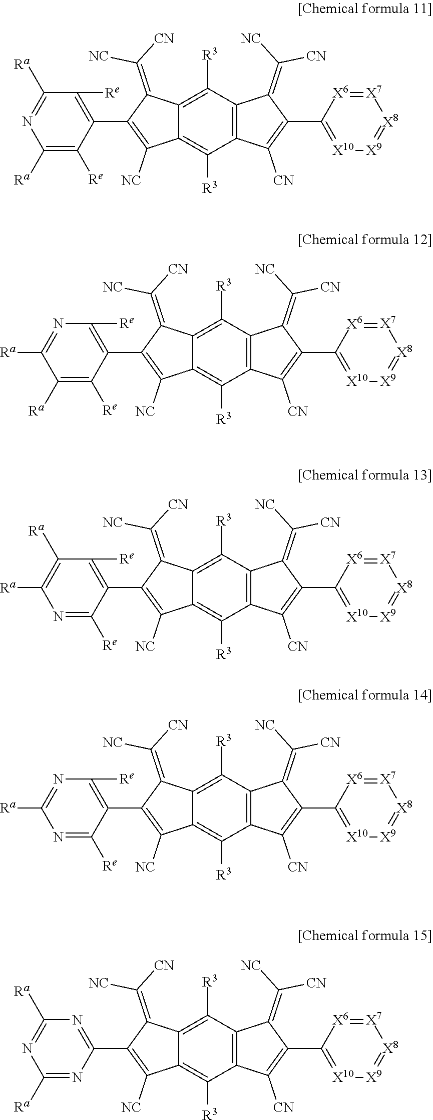
in chemical formula 6 to chemical formula 15, R^{a}, R³ and X⁶ to X¹⁰ are the same as R^{a}, R³ and X⁶ to X¹⁰ defined in chemical formula 1, and wherein R^{e} is each independently selected from among hydrogen; deuterium; tritium; halogen; a cyano group; a haloalkyl group of C₁-C₅₀; and a haloalkoxy group of C₁-C₃₀.

6. The organic light emitting element of claim 1, wherein the compound represented by chemical formula 1 is one or more of compounds below:

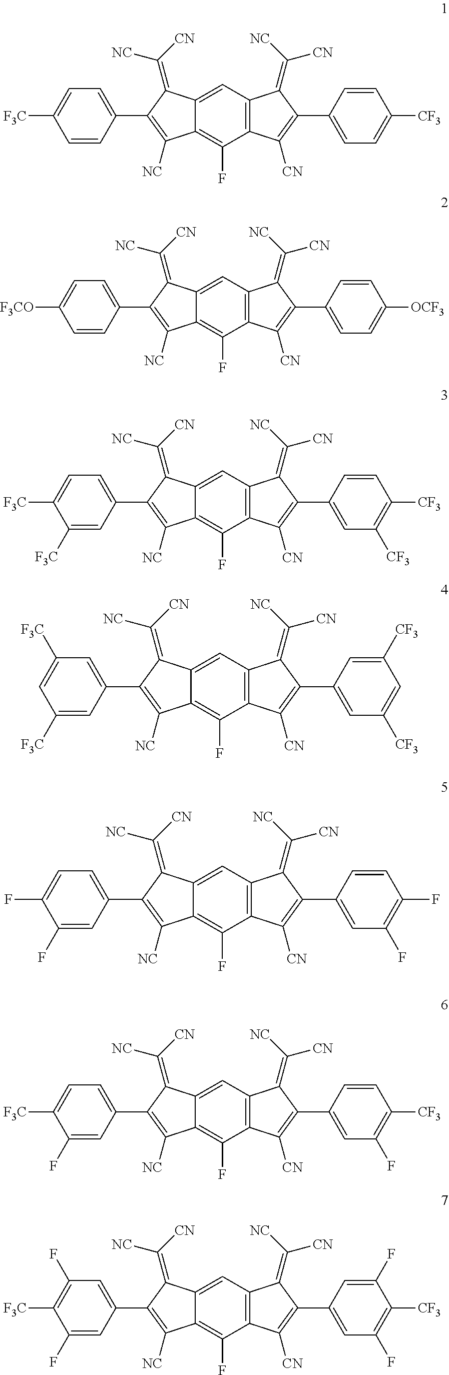

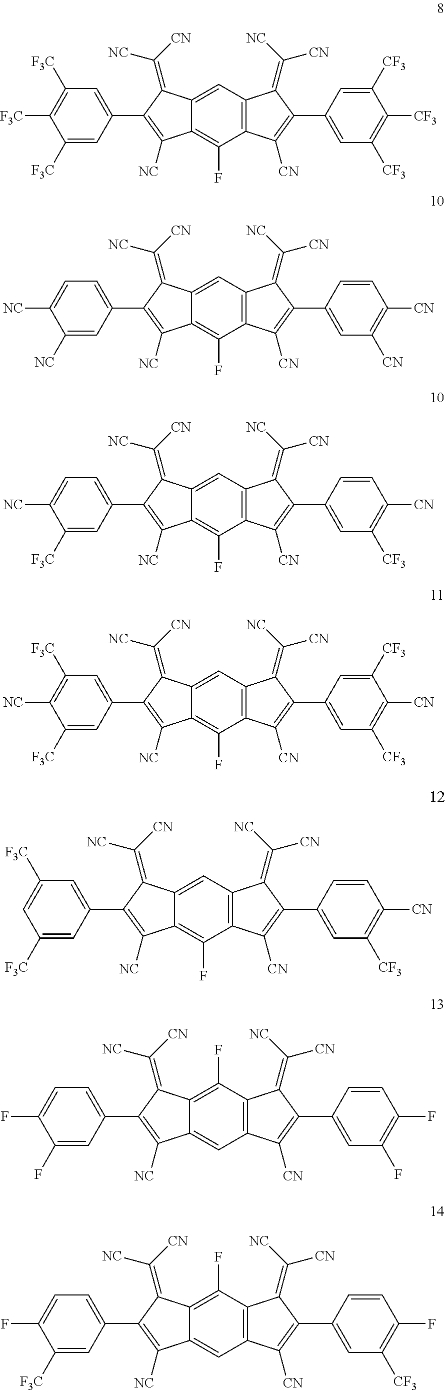

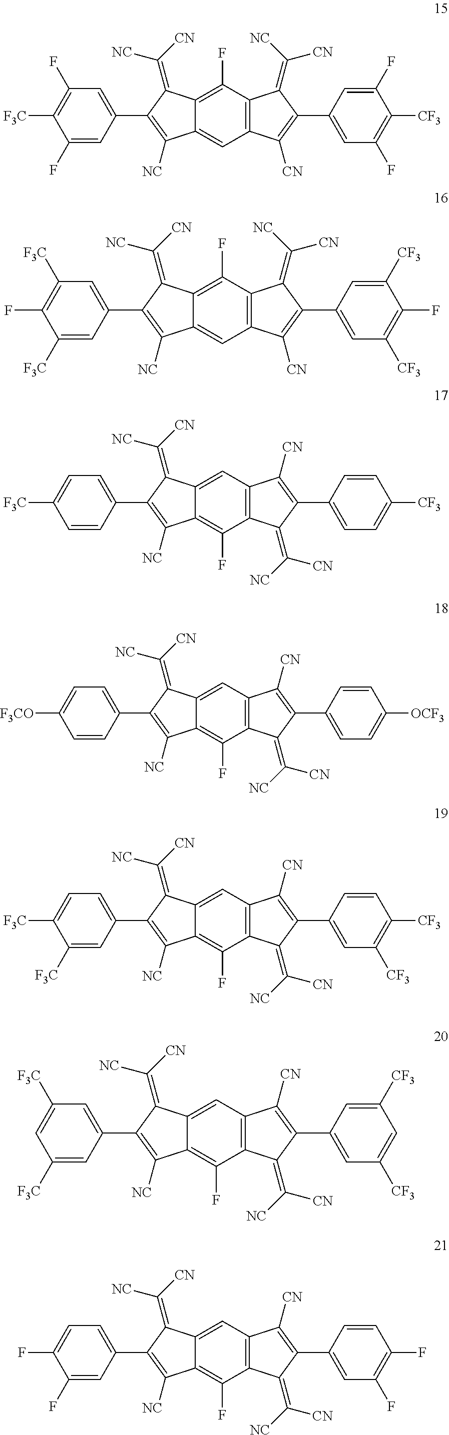

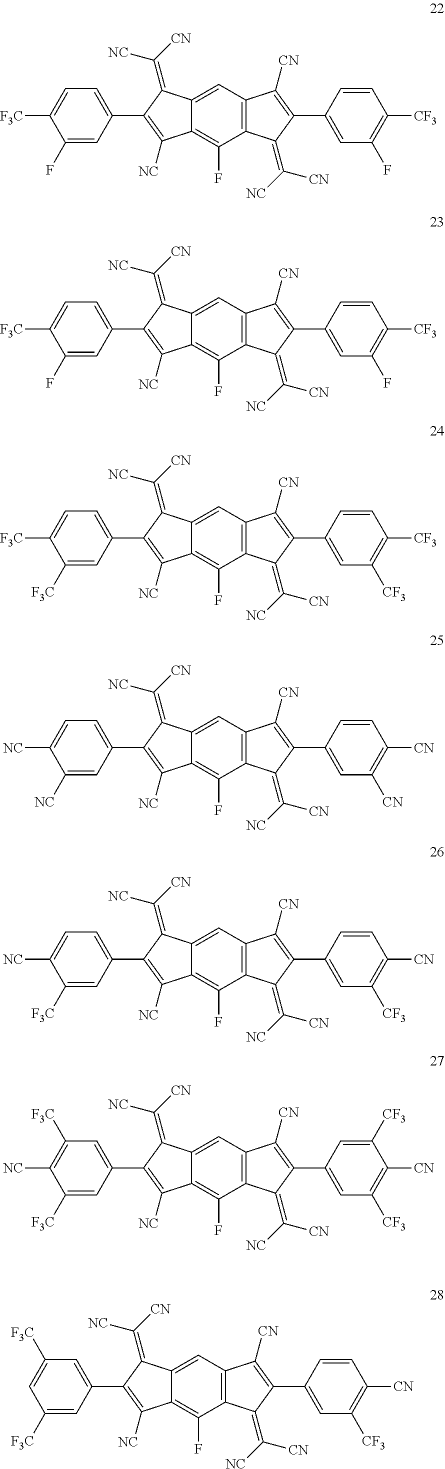

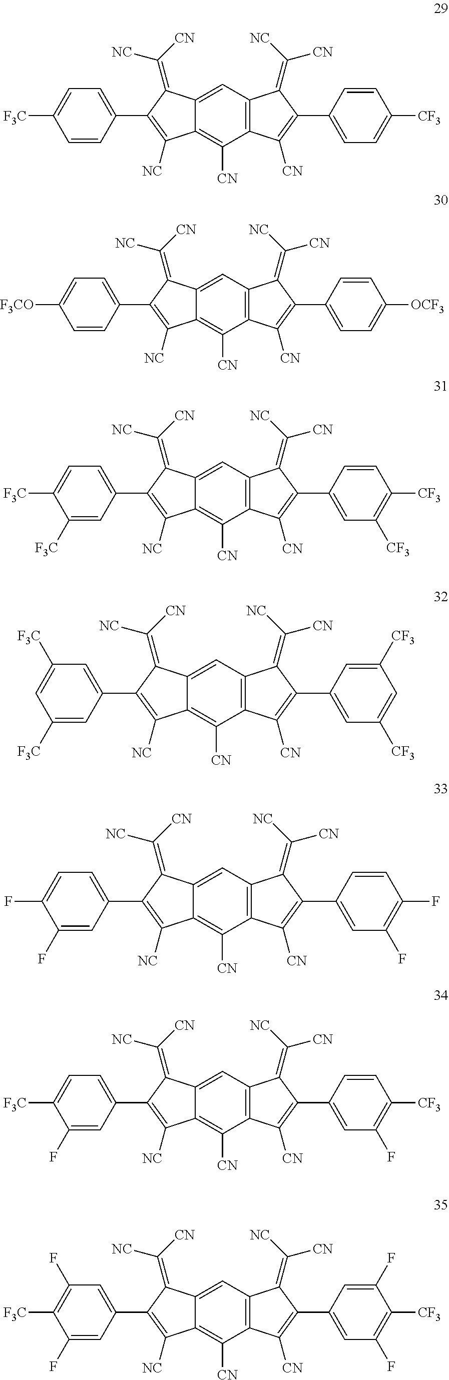

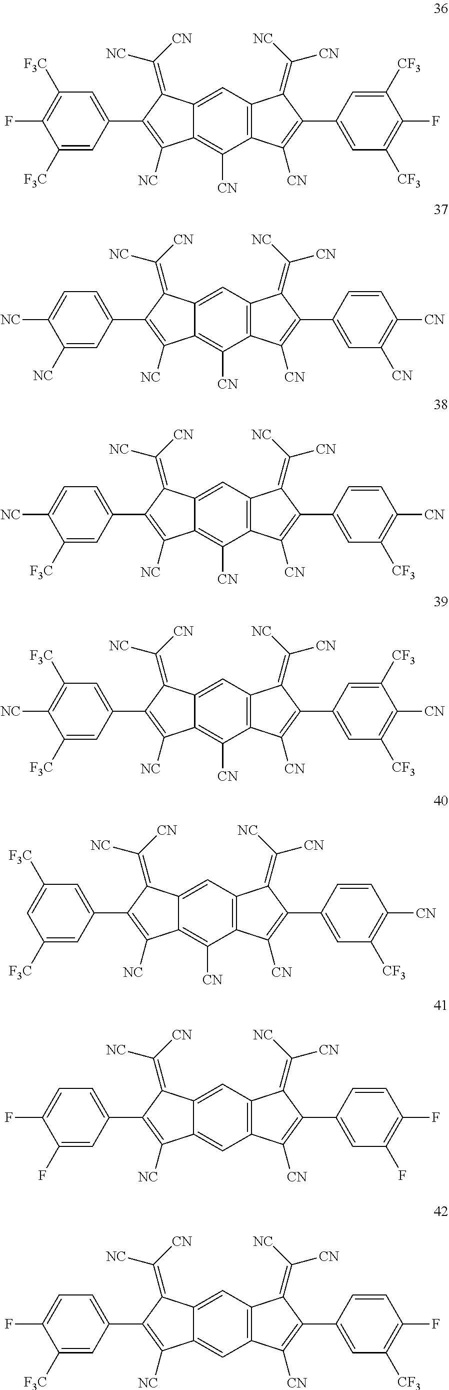

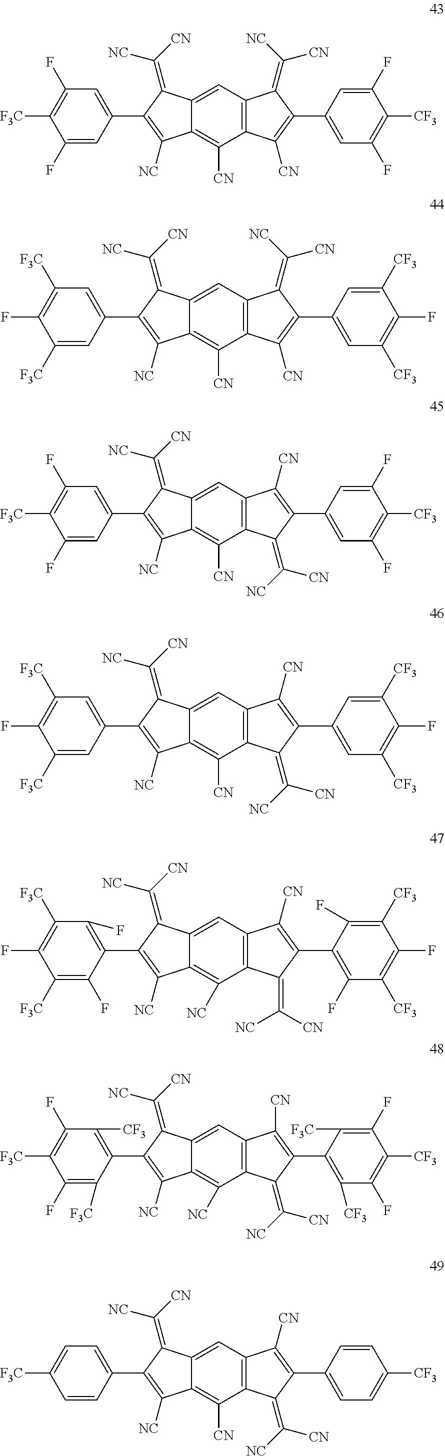

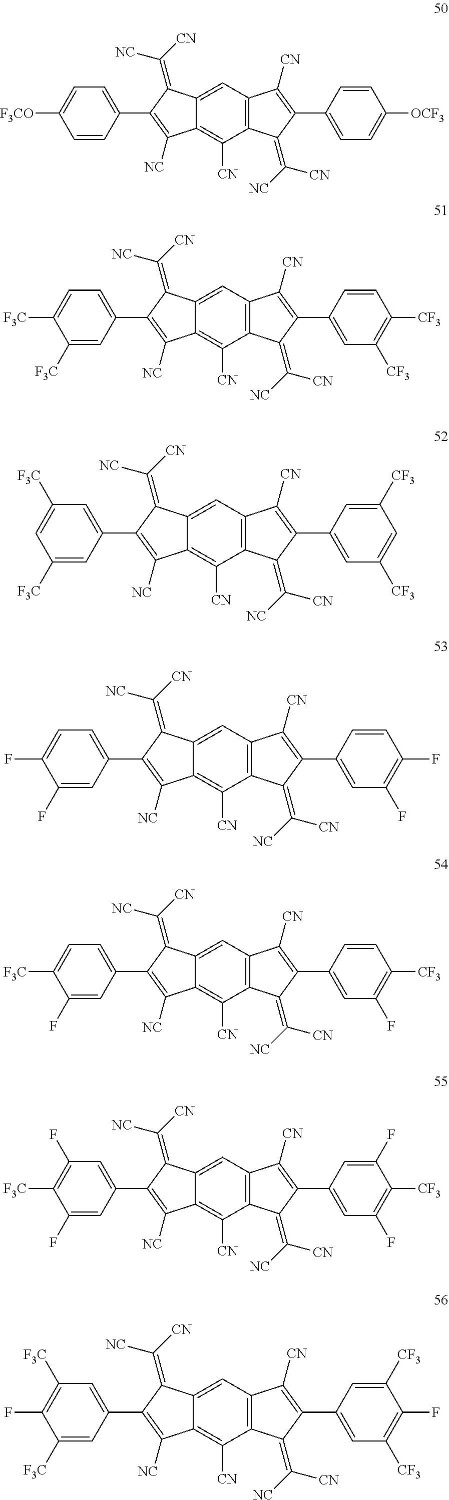

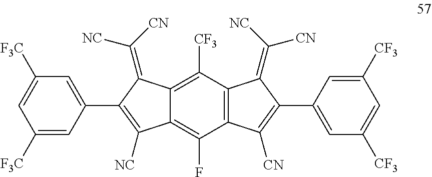 [SMILES: N#CC(C#N)=C1C(c2cc(C(F)(F)F)cc(C(F)(F)F)c2)=C(C#N)c2c(F)c3c(c(C(F)(F)F)c21)C(=C(C#N)C#N)C(c1cc(C(F)(F)F)cc(C(F)(F)F)c1)=C3C#N]

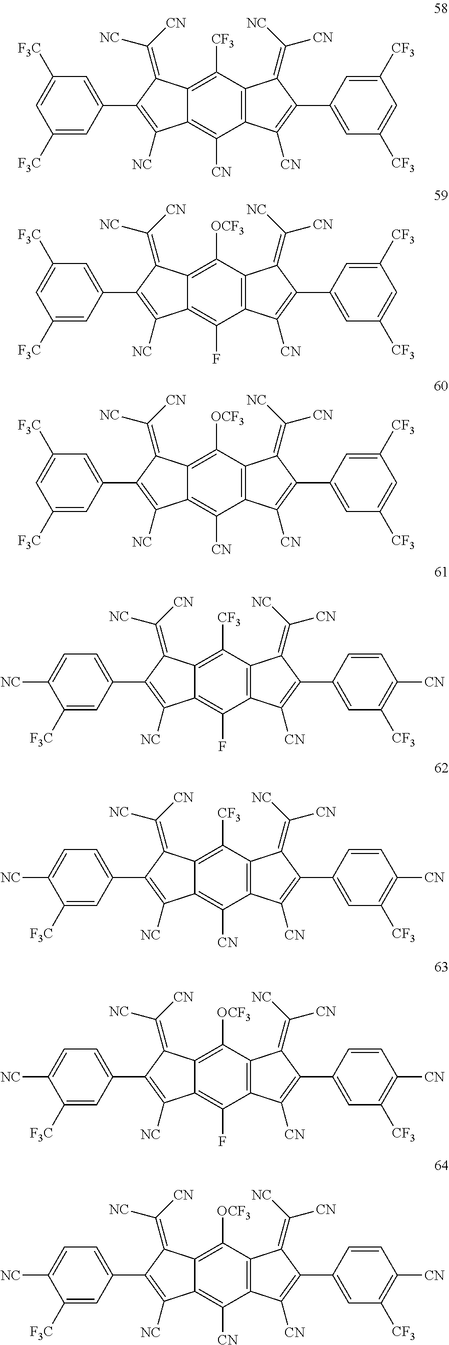

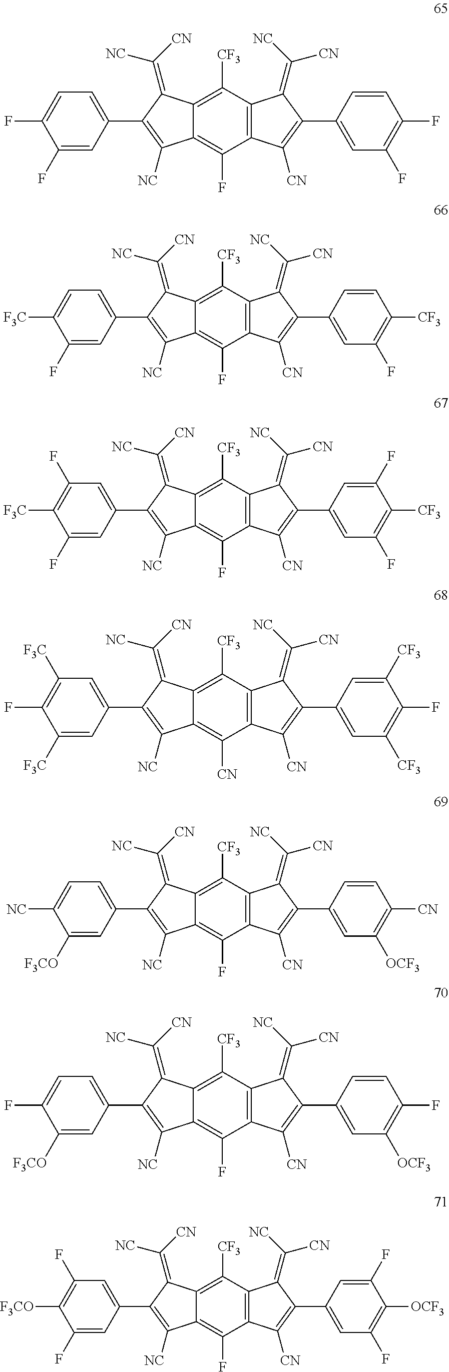

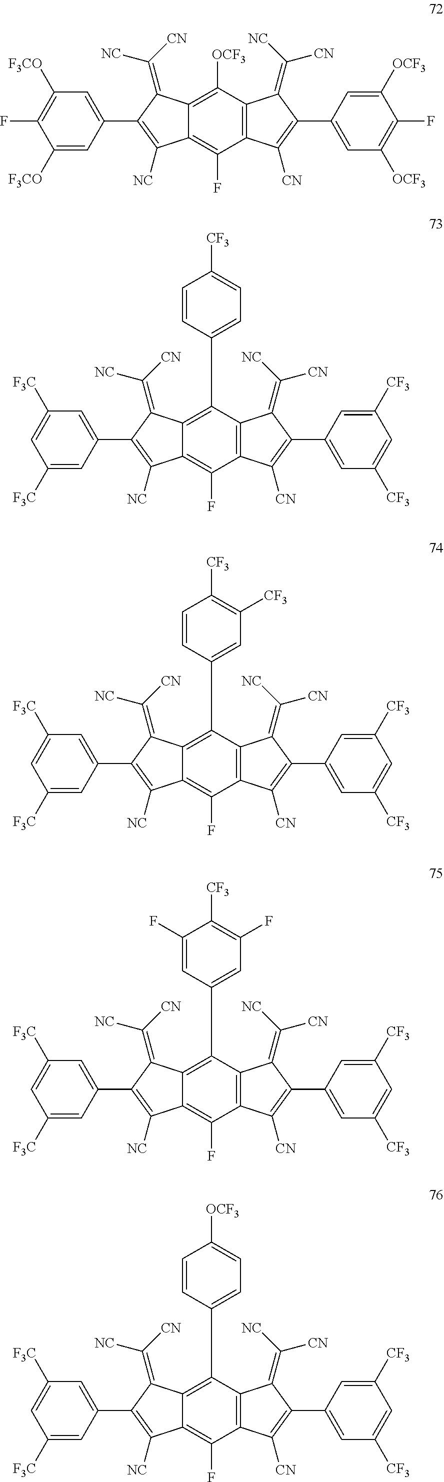

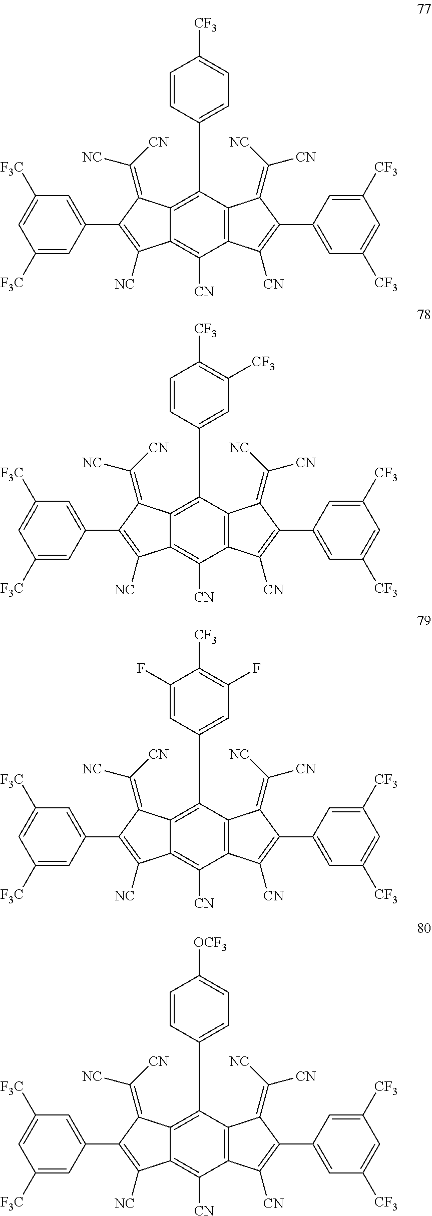

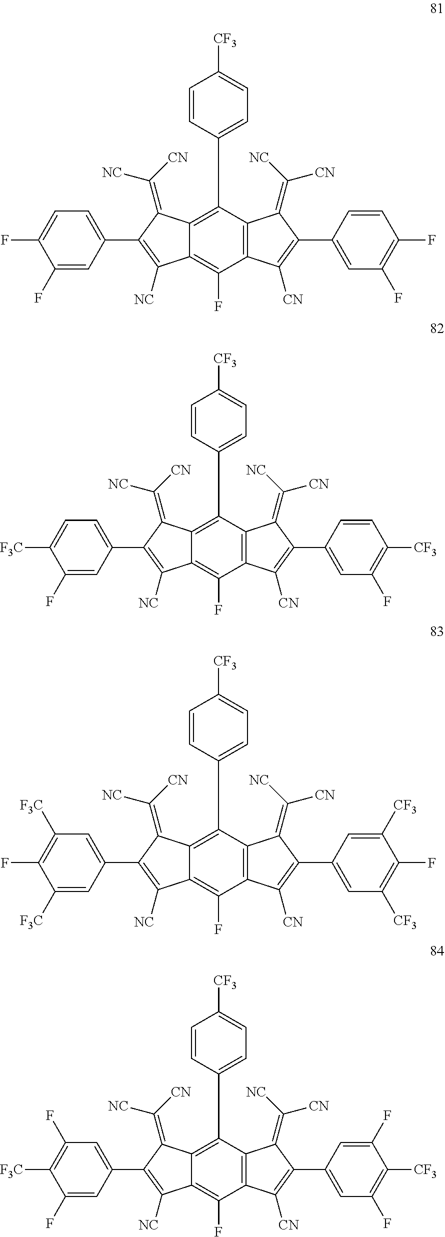

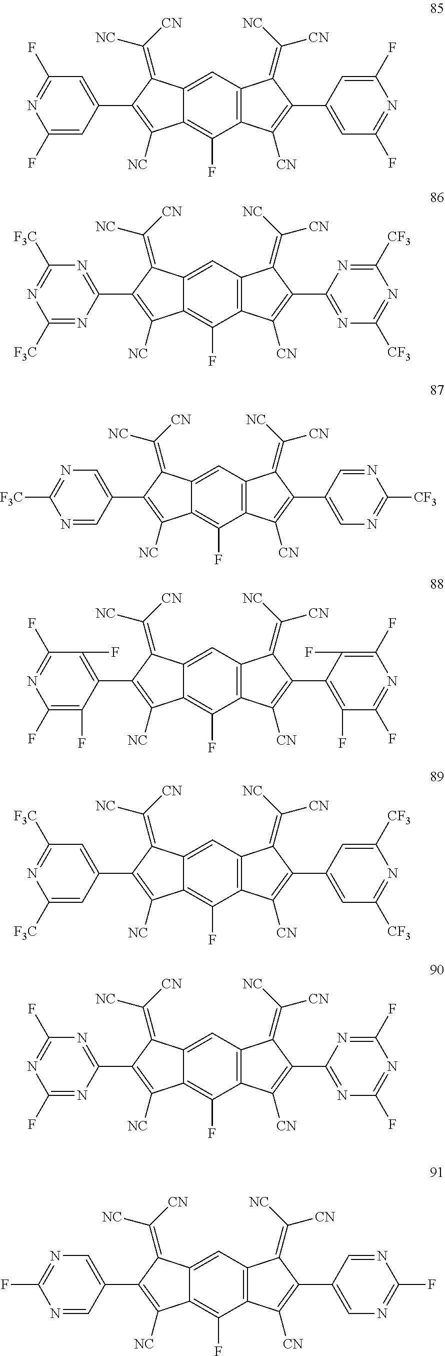

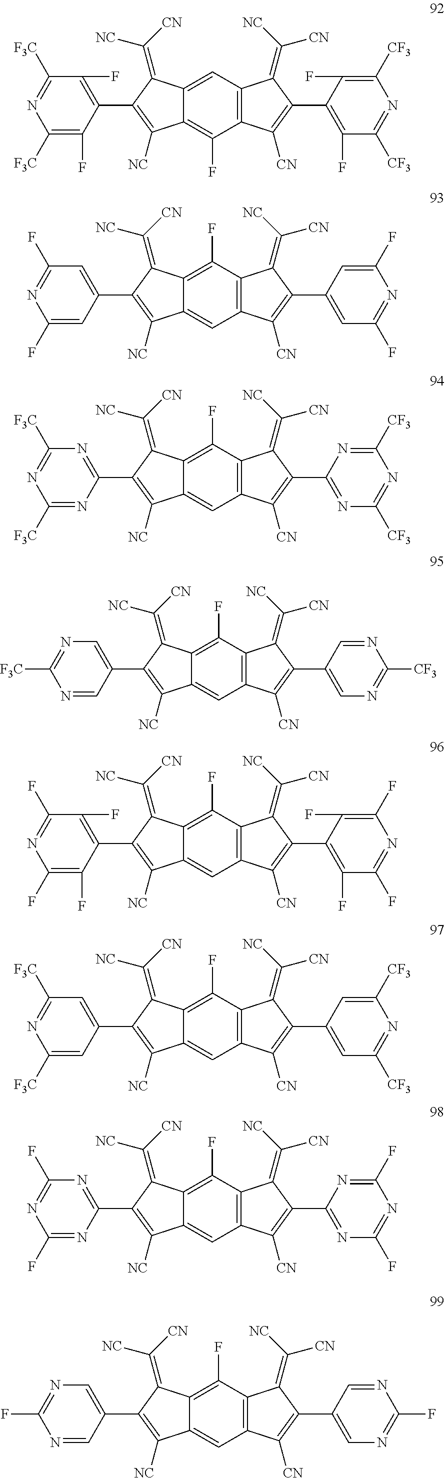

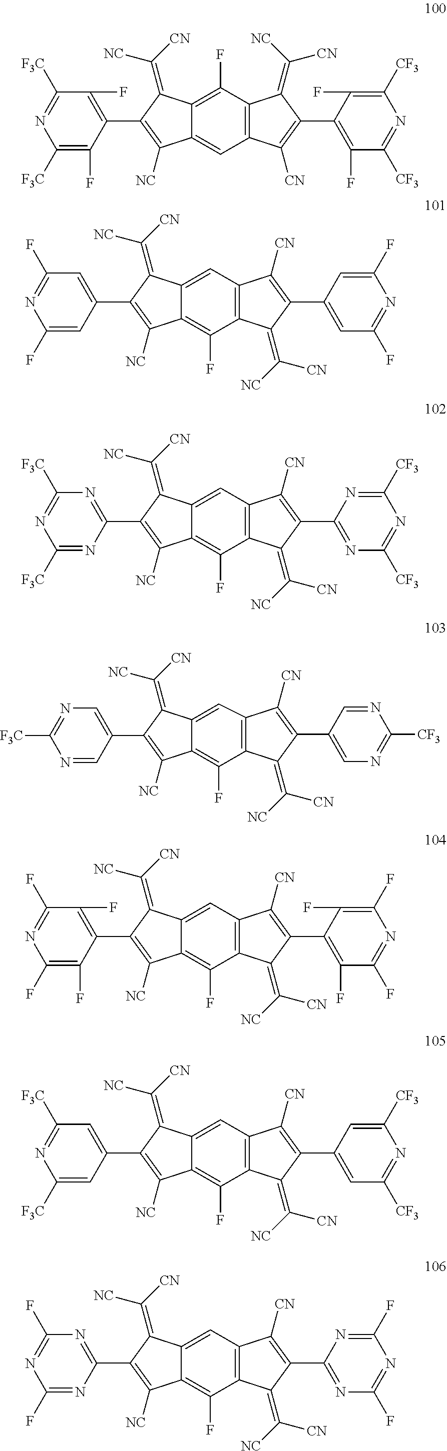

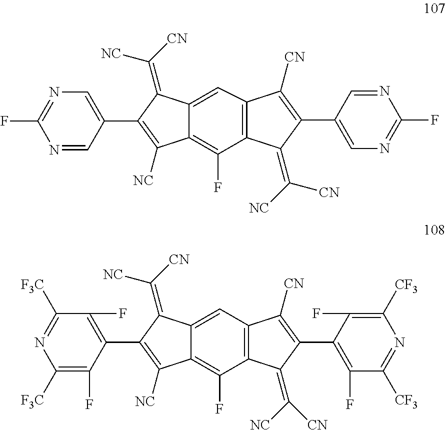 [SMILES: N#CC(C#N)=C1C(c2cnc(F)nc2)=C(C#N)c2c1cc1c(c2F)C(=C(C#N)C#N)C(c2cnc(F)nc2)=C1C#N ; N#CC(C#N)=C1C(c2c(F)c(C(F)(F)F)nc(C(F)(F)F)c2F)=C(C#N)c2c1cc1c(c2F)C(=C(C#N)C#N)C(c2c(F)c(C(F)(F)F)nc(C(F)(F)F)c2F)=C1C#N]

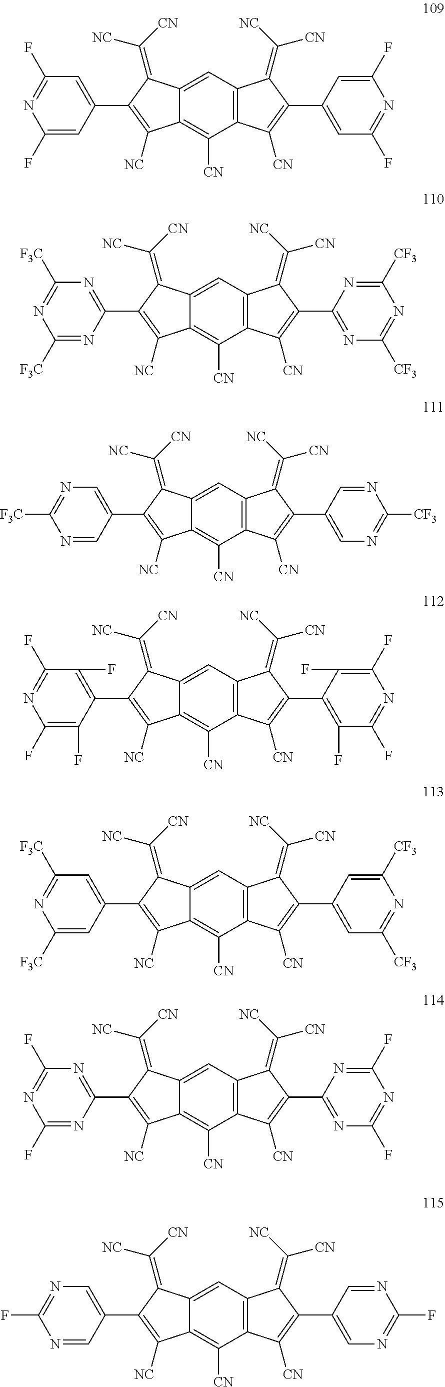

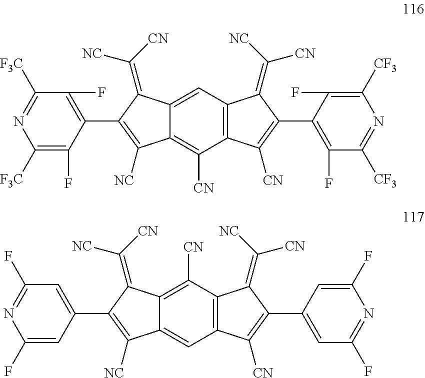 [SMILES: N#CC(C#N)=C1C(c2c(F)c(C(F)(F)F)nc(C(F)(F)F)c2F)=C(C#N)c2c1cc1c(c2C#N)C(C#N)=C(c2c(F)c(C(F)(F)F)nc(C(F)(F)F)c2F)C1=C(C#N)C#N ; N#CC(C#N)=C1C(c2cc(F)nc(F)c2)=C(C#N)c2cc3c(c(C#N)c21)C(=C(C#N)C#N)C(c1cc(F)nc(F)c1)=C3C#N]

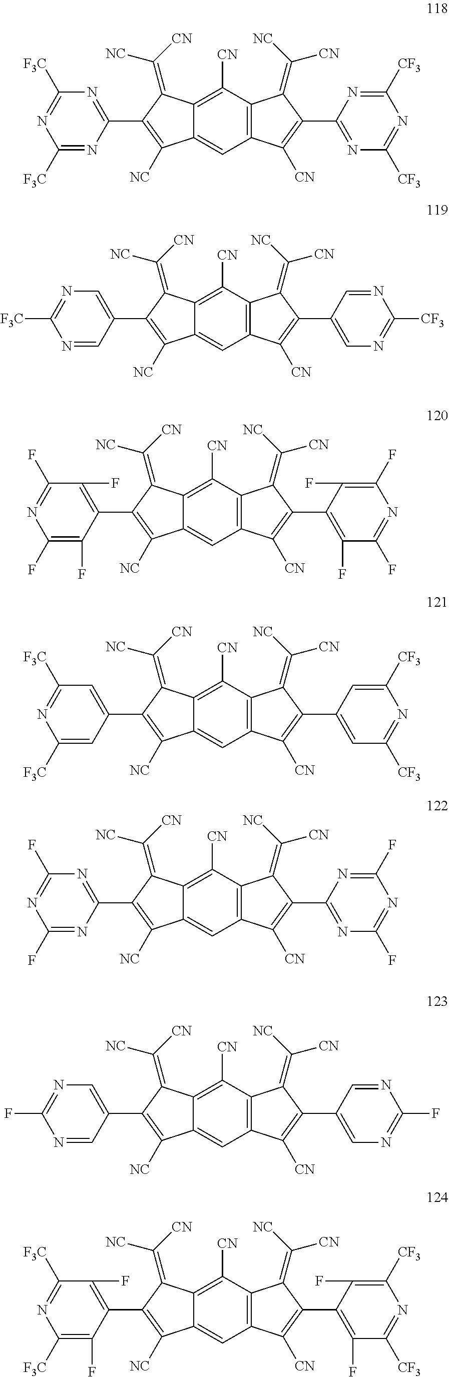

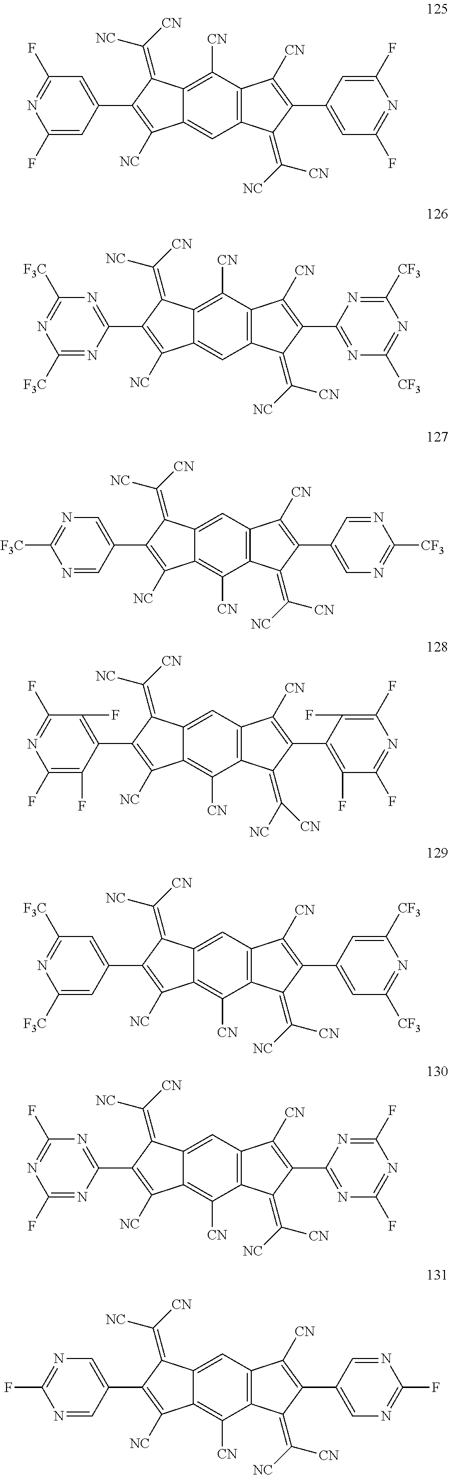

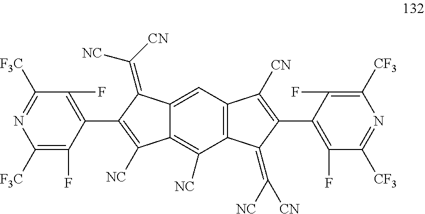 [SMILES: N#CC(C#N)=C1C(c2c(F)c(C(F)(F)F)nc(C(F)(F)F)c2F)=C(C#N)c2c1cc1c(c2C#N)C(=C(C#N)C#N)C(c2c(F)c(C(F)(F)F)nc(C(F)(F)F)c2F)=C1C#N]

7. The organic light emitting element of claim 1, wherein the organic material layer includes a first light emitting layer and a first layer, and wherein the first layer includes the compound.

8. The organic light emitting element of claim 7, wherein the first electrode is an anode electrode, and the second electrode is a cathode electrode, and wherein the first layer is positioned between the first electrode and the first light emitting layer.

9. The organic light emitting element of claim 8, wherein the first layer is a hole injection layer.

10. The organic light emitting element of claim 7, wherein the organic material layer further includes a second light emitting layer, and wherein the first layer is positioned between the first light emitting layer and the second light emitting layer.

11. The organic light emitting element of claim 10, wherein the first layer is a charge generation layer.

12. The organic light emitting element of claim 10, wherein the compound is a p dopant of the first layer.

13. The organic light emitting element of claim 10, wherein the organic material layer further includes a third light emitting layer and a charge generation layer positioned between the second light emitting layer and the third light emitting layer.

14. The organic light emitting element of claim 13, wherein the charge generation layer positioned between the second light emitting layer and the third light emitting layer includes the compound represented by chemical formula 1.

15. A display device comprising the organic light emitting element of claim 1.
